(19) United States
(12) Patent Application Publication
(10) Pub. No.: US 2011/0237533 A1
(43) Pub. Date: Sep. 29, 2011

(54) NOVEL POLYPHENOL COMPOUND

(75) Inventors: Kenzo NOMURA, (US); Yayoi Kanbayashi, (US)

(73) Assignee: AJINOMOTO CO.,INC., Chuo-ku (JP)

(21) Appl. No.: 13/078,108

(22) Filed: Apr. 1, 2011

Related U.S. Application Data
(63) Continuation of application No. PCT/JP2009/067190, filed on Oct. 1, 2009.

(30) Foreign Application Priority Data
Oct. 1, 2008 (JP) 2008-256899

(51) Int. Cl. A61K 31/7048; C07D 493/04; A61K 31/357; C07H 15/26; A61P 3/04; C12P 19/60; C12P 17/18

(52) U.S. Cl. 514/27; 549/358; 514/452; 536/18.1; 435/75; 435/119

(57) ABSTRACT

Compounds represented by formula (I)

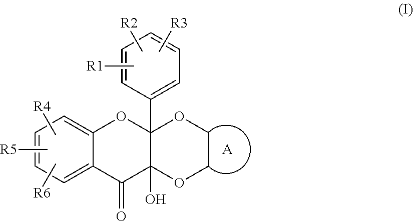

wherein the symbols are as defined in the description, exhibit a lipase inhibitory activity, and are useful for foods, drinks, and the like.

### CROSS REFERENCES TO RELATED APPLICATIONS

This application is a continuation of International Patent Application No. PCT/JP2009/067190, filed on Oct. 1, 2009, and claims priority to Japanese Patent Application No. 2008-256899, filed on Oct. 1, 2008, both of which are incorporated herein by reference in their entireties.

### BACKGROUND OF THE INVENTION

1. Field of the Invention

The present invention relates to novel polyphenol compounds having a lipase inhibitory activity. The present invention also relates to foods and drinks which contain such a compound and pharmaceutical compositions which contain such a compound. The present invention also relates to methods of producing such a compound.

2. Discussion of the Background

The increase of obesity caused by increased intake of high-fat diet, overeating due to stress, insufficient exercise and the like is posing problems. To prevent or reduce obesity, postprandial hyperlipemia and lipid dysbolism due to the intake of high-fat diet, it is useful to suppress the systemic absorption of diet-derived fat. Fat in ingested food is hydrolyzed by lipase secreted from the pancreas into the intestinal tract, and absorbed from the small intestine into the body. Therefore, if decomposition of the fat in foods can be suppressed by inhibiting the action of lipase, absorption of fat into the body can be suppressed, and the prophylaxis or improvement of obesity, postprandial hyperlipemia and lipid dysbolism caused by the intake of high-fat diet can be expected. Furthermore, improvement of abnormal glucose metabolism such as insulin resistance associated and increase of blood insulin concentration with visceral fat type obesity can also be expected.

In addition, lipase produced by human skin indigenous bacteria such as Propionibacterium acnes and the like decomposes fat in the sebum into glycerol and free fatty acid. It is known that free fatty acids contain a substance that exerts an adverse influence on the skin, which causes acne, comedone and the like, and free fatty acids are further decomposed to cause body odor. Thus, inhibition of the action of lipase is useful for the prophylaxis or improvement of dermatic diseases such as acne and the like and body odor.

As a food that inhibits absorption of neutral fats during eating, foods and drinks containing a catechin polymer having an action to inhibit pancreatic lipase as a main component (see, JP-A-2005-336117 and JP-A-2006-16367) and the like have been developed.

As for lipase inhibition, polyphenol is known to have activity, and plant-derived tannin (see, J. Agric. Food Chem. 2007 May 30; 55(11):4604-9), and flavonoids, particularly epigallocatechin gallate and epicatechin gallate having a galloyl group have been reported to have a strong lipase inhibitory activity and the like (see, J. Agric. Food Chem. 2005 Jun. 1; 53(11):4593-8).

When the intensity of lipase inhibitory activity of the polyphenols is compared, dimers and trimers produced by polymerization of flavonoids and the like have been reported to show stronger activity than flavonoid monomer (see, JP-A-2005-336117).

It is already well known that polyphenoloxydases are involved in the polymerization of polyphenol, and industrially widely applied to the polymerization reaction of phenols (see, JP-A-2006-180745). In addition, polyphenoloxydase is added to epigallocatechin and gallic acid and the mixture is also utilized for the manufacture method for producing epitheaflagallins useful as food (see, JP-A-2007-319140).

However, when the conventionally presented, the above-mentioned polyphenol having a lipase inhibitory activity is to be utilized, problems of quantitative and qualitative dispersion of the active constituents and the like occur depending on the collecting time, collecting area and the like since it is a natural product. Thus, it is difficult to stably ensure a certain level of activity intensity, and an operation such as component concentration and the like is necessary for the stable maintenance of activity intensity (see, WO2005/077384). On the other hand, even when conventional polyphenols are actually formulated into a supplement and the like and used by oral administration and the like, they are absorbed in the intestine before showing effects, and cannot effectively exhibit a lipase inhibitory effect in the intestine. As such, there are very few that are practically sufficiently satisfactory.

### SUMMARY OF THE INVENTION

Accordingly, it is one object of the present invention to provide novel polyphenol compounds having a superior lipase inhibitory action.

It is another object of the present invention to provide novel polyphenol compounds having a superior lipase inhibitory action, which are not easily absorbed in the intestine.

It is another object of the present invention to provide novel polyphenol compounds having a superior lipase inhibitory action, which have a high safety.

It is another object of the present invention to provide novel polyphenol compounds having a superior lipase inhibitory action which can be administered orally.

It is another object of the present invention to provide novel methods for preparing such a polyphenol compound.

It is another object of the present invention to provide novel foods, drinks, and pharmaceutical compositions which contain such a polyphenol compound.

These and other objects, which will become apparent during the following detailed description, have been achieved by the inventors' discovery that a specific reaction wherein a flavonol compound which is a naturally occurring polyphenol component is combined with one or more kinds of component selected from the group consisting of caffeic acid, gallic acid, chlorogenic acid, dicaffeoylquinic acid, catechin, gallocatechin, catechin gallate and gallocatechin gallate and polyphenoloxydase is added to allow reaction, whereby catechol is bonded to the 2-position and the 3-position of the flavonol compound, affords useful polyphenol compounds.

Furthermore, the present inventors have found that the specific reaction produces a novel compound having a skeleton seldom seen in known flavonoid dimers, a superior lipase inhibitory activity, and a structure not easily absorbed in the intestine.

Accordingly, the present invention provides:

(1) a compound represented by the formula (I)

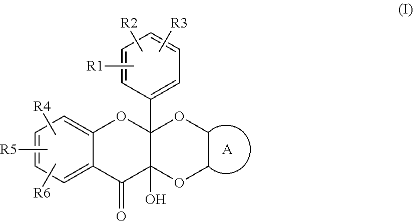

in the formula (I),
R1, R2, R3, R4, R5 and R6 may be the same or different and each is
a hydrogen atom,
a hydroxyl group,
alkoxy group, or
a sugar residue consisting of 1 or 2 sugars selected from the group consisting of glucose, rhamnose, fructose and galactose;
ring A is a ring selected from the group consisting of the following formulas (II-a)-(II-i)

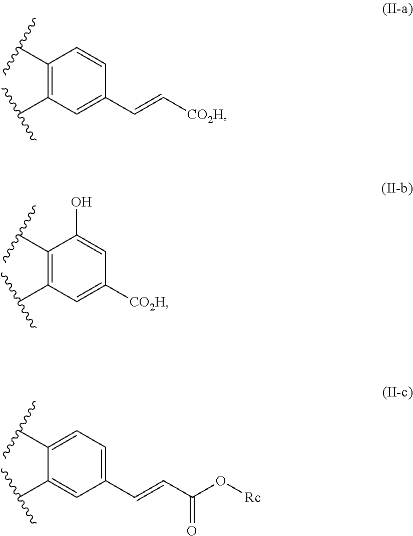

in the formula (II-c),
Rc is a group represented by the following formula

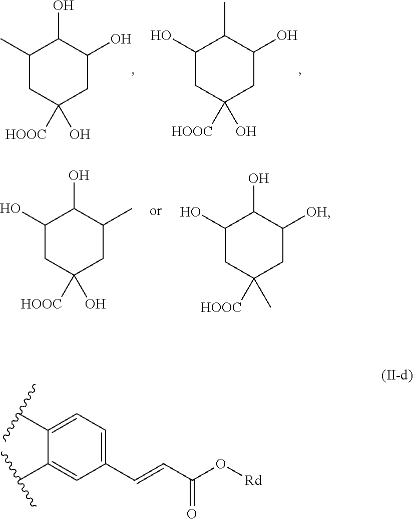

in the formula (II-d),
Rd is a group represented by the following formula

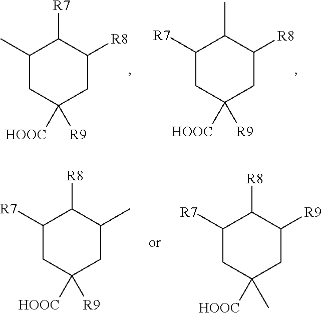

wherein
any one group of R7, R8 and R9 is a group represented by the formula:

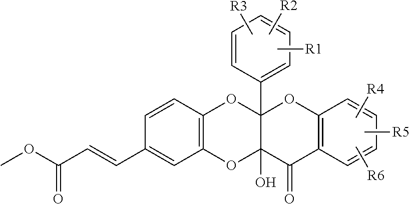

wherein R1, R2, R3, R4, R5 and R6 are as defined above, a group represented by the formula:

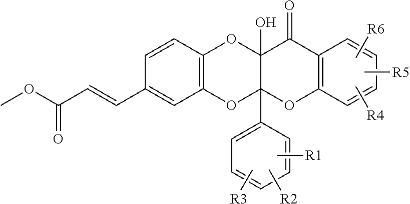

wherein R1, R2, R3, R4, R5 and R6 are as defined above, or a group represented by the formula:

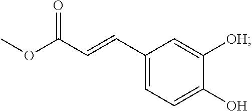

and
two groups of the remaining R7, R8 and R9 are hydroxyl groups,

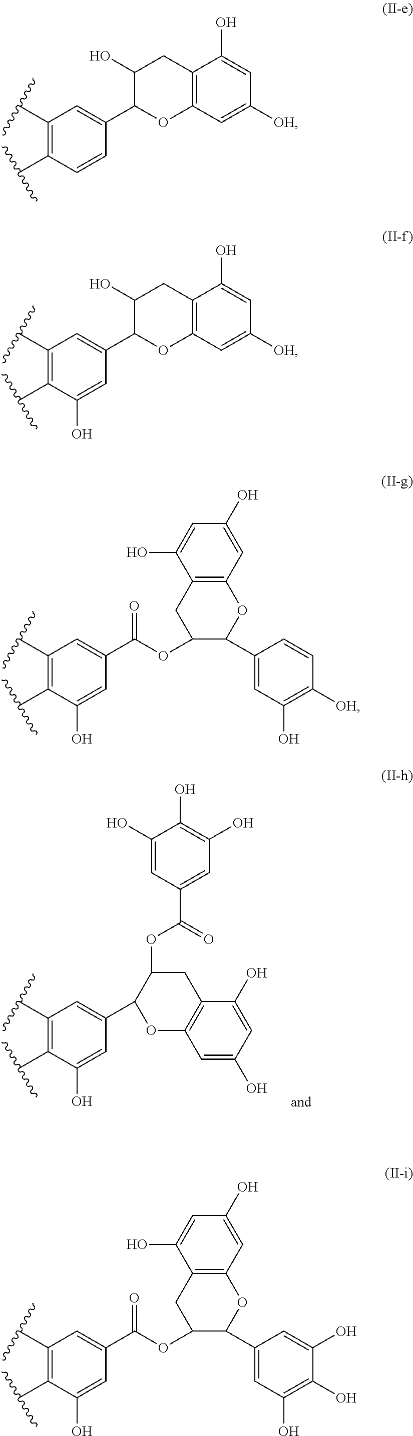

wherein the above-mentioned rings are each optionally condensed with the adjacent dioxane ring in any orientation (hereinafter sometimes to be referred to as compound (I) or the compound of the present invention);

(2) the compound of the above-mentioned (1), wherein R1, R2, R3, R4, R5 and R6 may be the same or different and each is

a hydrogen atom,
a hydroxyl group,
a methoxy group, or
a sugar residue consisting of 1 or 2 sugars selected from the group consisting of glucose, rhamnose, fructose and galactose;

(3) the compound of the above-mentioned (1), which is represented by the formula (I′)

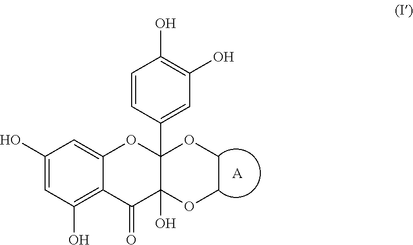

wherein ring A is a ring selected from the group consisting of the following formulas (II-a′)-(II-g′)

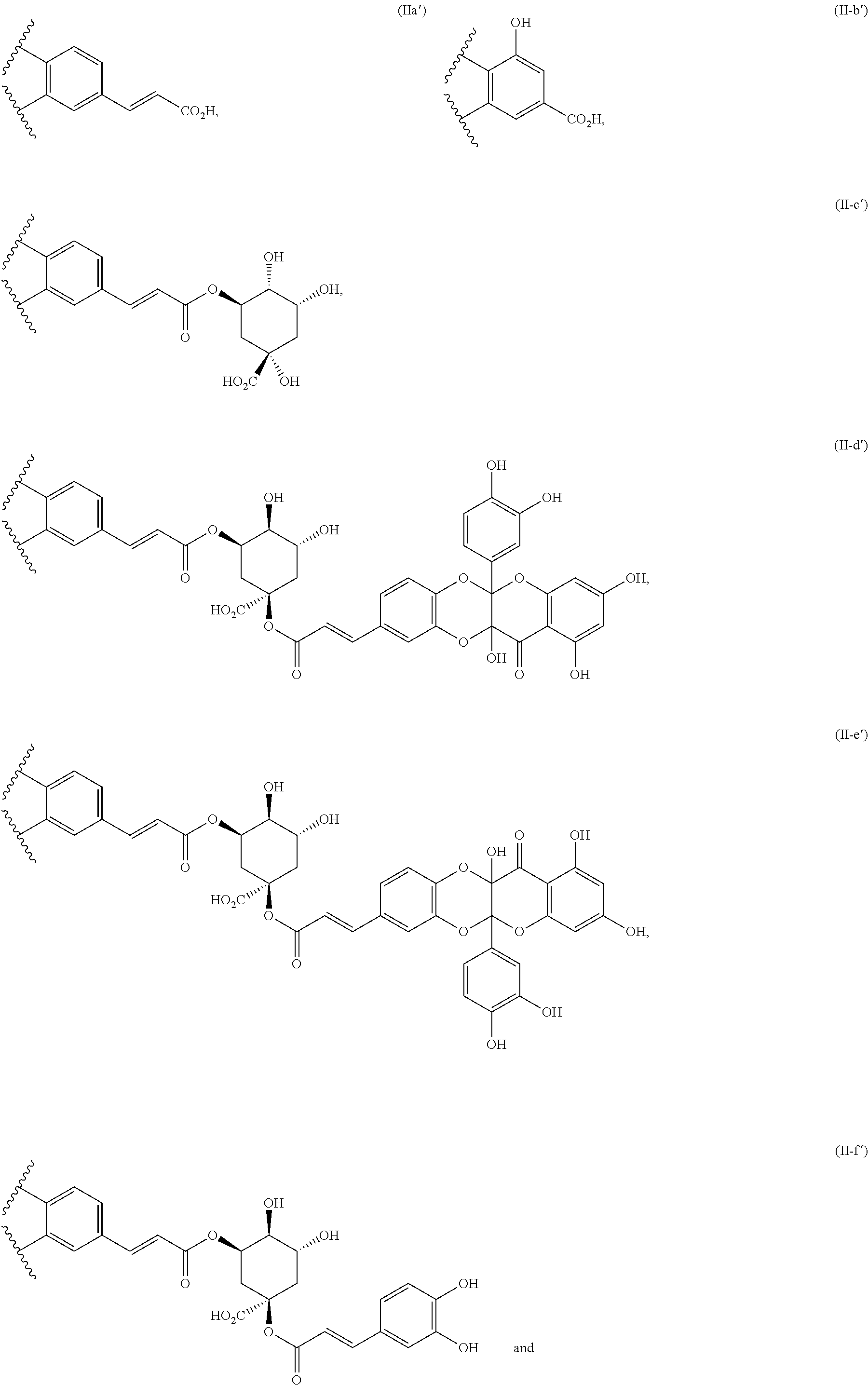

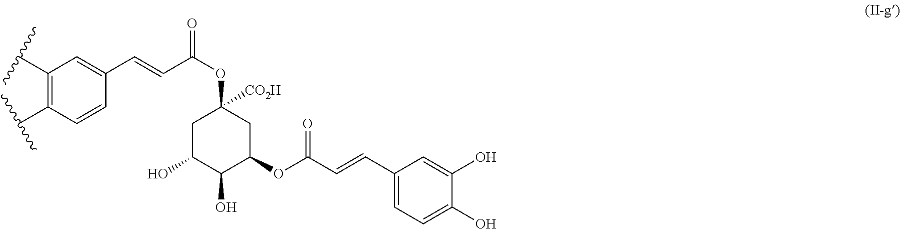

wherein the above-mentioned rings are each optionally condensed with the adjacent dioxane ring in any orientation;

(4) a resultant product obtained by reacting one or more kinds of flavonol compounds with one or more kinds of components selected from the group consisting of caffeic acid, gallic acid, chlorogenic acid, dicaffeoylquinic acid, catechin, gallocatechin, catechin gallate and gallocatechin gallate in the presence of polyphenoloxydase;

(5) the resultant product of the above-mentioned (4) wherein one or more kinds of flavonol compounds and one or more kinds of components selected from the group consisting of caffeic acid, gallic acid, chlorogenic acid, dicaffeoylquinic acid, catechin, gallocatechin, catechin gallate and gallocatechin gallate are extracted from natural materials;

(6) a resultant product obtained by reacting one or more kinds of flavonol compounds represented by the formula (III)

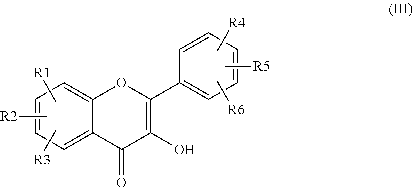

wherein R1, R2, R3, R4, R5 and R6 are as defined in the above-mentioned (1), with
one or more kinds of components selected from the group consisting of a compound represented by the following structural formula

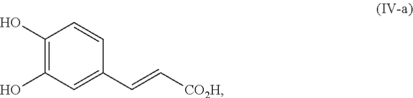

a compound represented by

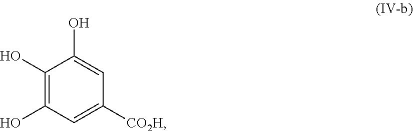

a compound represented by

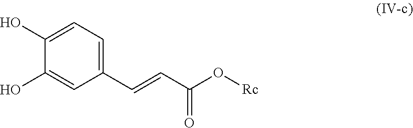

in the formula (IV-c),
Rc is a group represented by the following formula

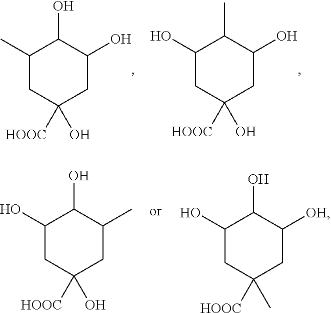

a compound represented by

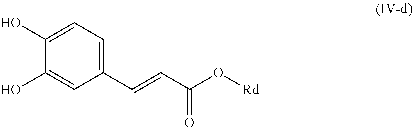

in the formula (IV-d),
Rd is a group represented by the following formula

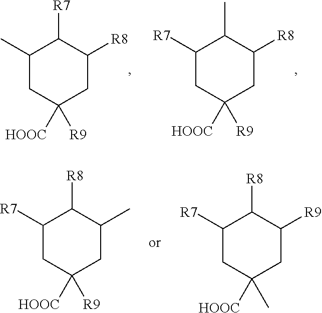

wherein
any one group of R7, R8 and R9 is a group represented by the formula:

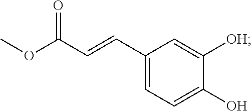

and
two groups of the remaining R7, R8 and R9 are hydroxyl groups, a compound represented by

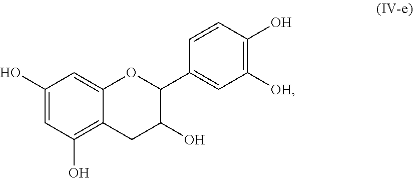

a compound represented by

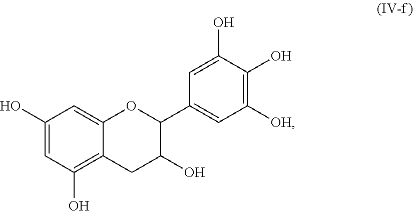

a compound represented by

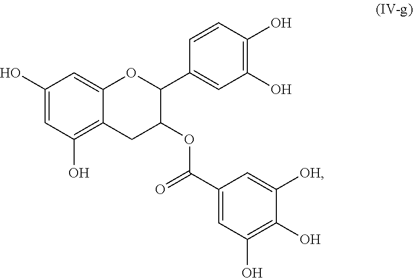

and
a compound represented by

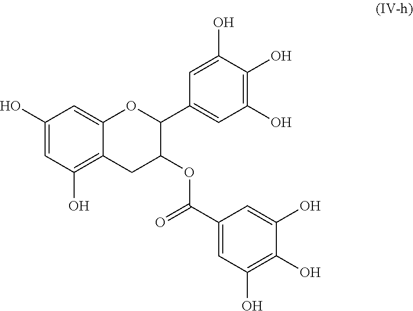

in the presence of polyphenoloxydase (hereinafter the resultant product of the above-mentioned (4) and the resultant product of (6) are sometimes to be generically referred to as the resultant product of the present invention);

(7) the resultant product of the above-mentioned (4), which is obtained by reacting quercetin with one or more kinds of components selected from the group consisting of caffeic acid, gallic acid, chlorogenic acid and dicaffeoylquinic acid in the presence of polyphenoloxydase;

(8) a composition for food or drink comprising the compound of the above-mentioned (1) or the resultant product of the above-mentioned (4) or (6);

(9) the composition for food or drink of the above-mentioned (8), which is a tea drink, beverage or health food;

(10) the composition for food or drink of the above-mentioned (8), which is a food or drink for dieting;

(11) the composition for food or drink of the above-mentioned (8), which is a food or drink for inhibiting lipase activity;

(12) the composition for food or drink of the above-mentioned (8), which is a food or drink for suppressing fat absorption;

(13) the composition for food or drink of the above-mentioned (8), which is a food or drink for the improvement or prophylaxis of postprandial hyperlipemia, lipid dysbolism, obesity or glucose metabolism disorder;

(14) use of the compound of the above-mentioned (1) or the resultant product of the above-mentioned (4) or (6) as a food or drink;

(15) a method of inhibiting lipase in a subject, comprising giving an effective amount of the compound of the above-mentioned (1) or the resultant product of the above-mentioned (4) or (6) to the subject;

(16) a method of suppressing fat absorption in a subject, comprising giving an effective amount of the compound of the above-mentioned (1) or the resultant product of the above-mentioned (4) or (6) to the subject;

(17) a method of producing the compound of the above-mentioned (1), comprising subjecting one or more kinds of flavonol compounds represented by the formula (III)

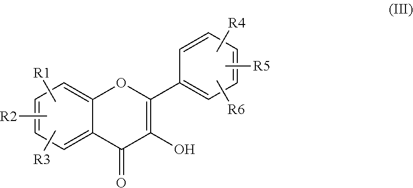

wherein R1, R2, R3, R4, R5 and R6 are as defined in the above-mentioned (1)
with one or more kinds of compounds selected from the group consisting of
a compound represented by the following structural formula

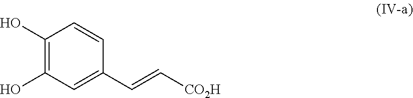

(i.e., caffeic acid), a compound represented by

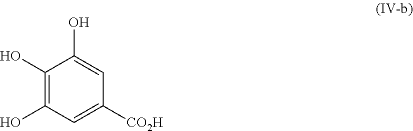

(i.e., gallic acid), a compound represented by

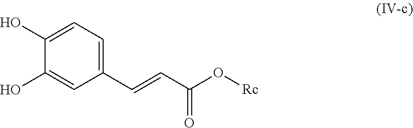

in the formula (IV-c),
Rc is a group represented by the following formula

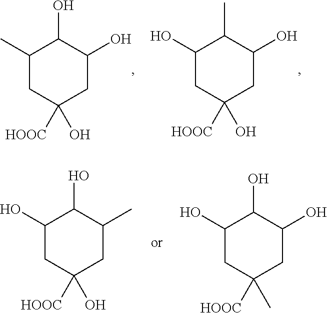

(i.e., chlorogenic acid), a compound represented by

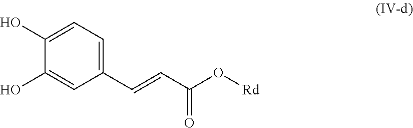

in the formula (IV-d),
Rd is a group represented by the following formula

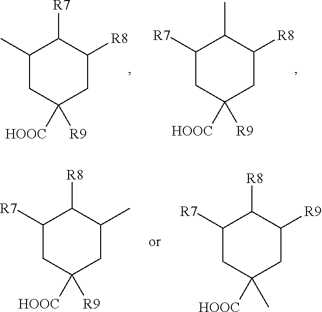

wherein
any one group of R7, R8 and R9 is a group represented by the formula:

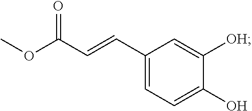

and
two groups of the remaining R7, R8 and R9 are hydroxyl groups (i.e., dicaffeoylquinic acid), a compound represented by

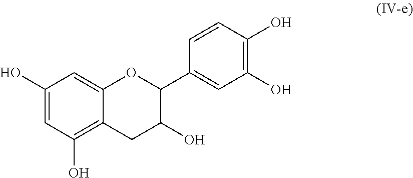

(i.e., catechin), a compound represented by

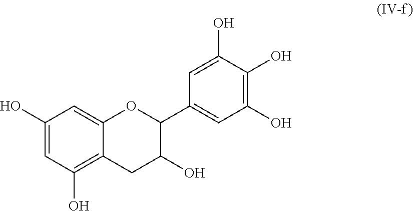

(i.e., gallocatechin), a compound represented by

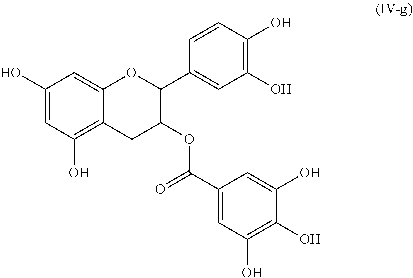

(i.e., catechin gallate), and a compound represented by

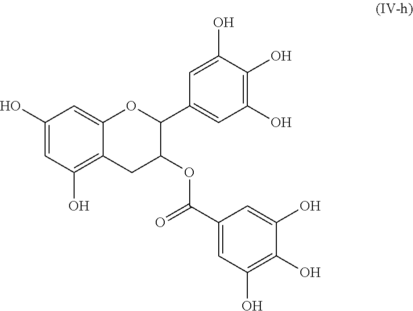

(i.e., gallocatechin gallate), to an oxidation polymerization reaction in the presence of polyphenoloxydase;

(18) a method of producing the compound of the above-mentioned (3), comprising subjecting one or more kinds of flavonol compounds represented by the formula (III′)

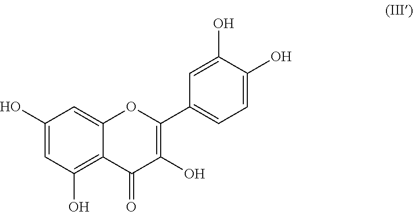

(i.e., quercetin), and one or more kinds of compounds selected from the group consisting of a compound represented by the following structural formula

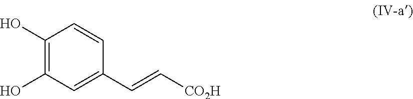

(i.e., caffeic acid), a compound represented by

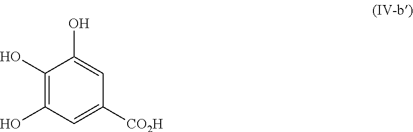

(i.e., gallic acid), a compound represented by

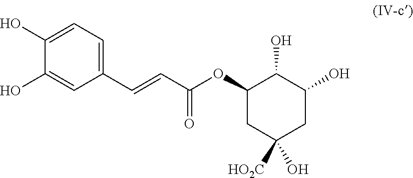

(i.e., chlorogenic acid), and a compound represented by

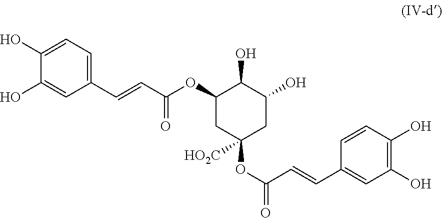

(i.e., dicaffeoylquinic acid), to an oxidation polymerization reaction in the presence of polyphenoloxydase; and

(19) a pharmaceutical composition comprising the compound of the above-mentioned (1) or the resultant product of the above-mentioned (4) or (6);

(20) the pharmaceutical composition of the above-mentioned (19), which is a lipase inhibitor;

(21) the pharmaceutical composition of the above-mentioned (19), which is a fat absorption inhibitor;

(22) the pharmaceutical composition of the above-mentioned (19), which is an agent for the prophylaxis or treatment of postprandial hyperlipemia, lipid dysbolism, obesity or glucose metabolism disorder;

(23) the pharmaceutical composition of the above-mentioned (19), which is an agent for the prophylaxis or treatment of acne;

(24) the pharmaceutical composition of the above-mentioned (19), which is an agent for the prophylaxis or treatment of body odor;

(25) use of the compound of the above-mentioned (1) or the resultant product of the above-mentioned (4) or (6) for the production of a lipase inhibitor;

(26) use of the compound of the above-mentioned (1) or the resultant product of the above-mentioned (4) or (6) for the production of a fat absorption inhibitor; and the like.

In the above-mentioned formula (I), “wherein the above-mentioned rings are each optionally condensed with the adjacent dioxane ring in any orientation” means, for example, when ring A is a ring represented by the formula (II-a)

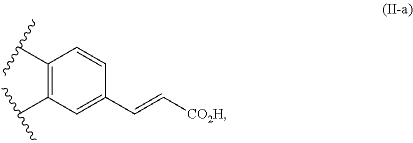

the form of binding of the formula (II-a) to the dioxane ring in the formula (I) is at the position shown in the embodiment of

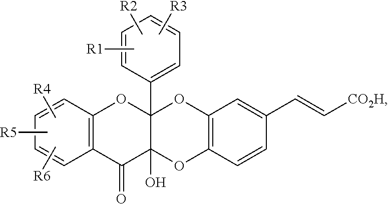

or at the position shown in the embodiment of

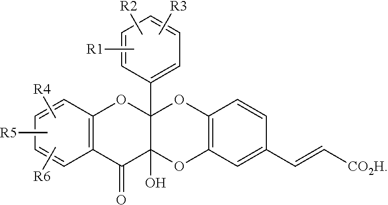

According to the present invention, a novel compound superior in a lipase inhibitory action can be provided.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows an HPLC chart of sample solutions after reaction, which contain (compound 1) or (compound 2).

FIG. 2-1 shows a mass spectrum of (compound 1) or (compound 2), which was obtained from the fraction at HPLC retention time of 42.01 minutes.

FIG. 2-2 shows a mass spectrum of (compound 1) or (compound 2), which was obtained from the fraction at HPLC retention time of 43.16 minutes.

FIG. 3-1 shows a UV spectrum of (compound 1) or (compound 2), which was obtained from the fraction at HPLC retention time of 42.01 minutes.

FIG. 3-2 shows a UV spectrum of (compound 1) or (compound 2), which was obtained from the fraction at HPLC retention time of 43.16 minutes.

FIG. 4 shows an HPLC chart of sample solutions after reaction, which contain (compound 3) or (compound 4).

FIG. 5-1 shows a mass spectrum of (compound 3) or (compound 4), which was obtained from the fraction at HPLC retention time of 27.2 minutes.

FIG. 5-2 shows a mass spectrum of (compound 3) or (compound 4), which was obtained from the fraction at HPLC retention time of 28.3 minutes.

FIG. 6-1 shows a UV spectrum of (compound 3) or (compound 4), which was obtained from the fraction at HPLC retention time of 27.2 minutes.

FIG. 6-2 shows a UV spectrum of (compound 3) or (compound 4), which was obtained from the fraction at HPLC retention time of 28.3 minutes.

FIG. 7 shows an HPLC chart of sample solutions after reaction, which contain (compound 5) or (compound 6).

FIG. 8-1 shows a mass spectrum of (compound 5) or (compound 6), which was obtained from the fraction at HPLC retention time of 33.7 minutes.

FIG. 8-2 shows a mass spectrum of (compound 5) or (compound 6), which was obtained from the fraction at HPLC retention time of 34.4 minutes.

FIG. 9-1 shows a UV spectrum of (compound 5) or (compound 6), which was obtained from the fraction at HPLC retention time of 33.7 minutes.

FIG. 9-2 shows a UV spectrum of (compound 5) or (compound 6), which was obtained from the fraction at HPLC retention time of 34.4 minutes.

FIG. 10-1 shows an HPLC chart of sample solutions after reaction, which contain a compound group from (compound 7) to (compound 10) and a compound group from (compound 11) to (compound 14).

FIG. 10-2 shows a chromatogram of a compound group from (compound 11) to (compound 14) in m/z817 selective ion detection mode.

FIG. 10-3 shows a chromatogram of a compound group from (compound 7) to (compound 10) in m/z1117 selective ion detection mode.

FIG. 11-1 shows a UV spectrum of any compound from (compound 11) to (compound 14), which was obtained from the fraction at HPLC retention time of 35.0 minutes.

FIG. 11-2 shows a UV spectrum of any compound from (compound 11) to (compound 14), which was obtained from the fraction at HPLC retention time of 35.5 minutes.

FIG. 11-3 shows a UV spectrum of any compound from (compound 11) to (compound 14), which was obtained from the fraction at HPLC retention time of 35.9 minutes.

FIG. 11-4 shows a UV spectrum of any compound from (compound 11) to (compound 14), which was obtained from the fraction at HPLC retention time of 36.2 minutes.

FIG. 11-5 shows a UV spectrum of any compound from (compound 7) to (compound 10), which was obtained from the fraction at HPLC retention time of 40.8 minutes.

FIG. 11-6 shows a UV spectrum of any compound from (compound 7) to (compound 10), which was obtained from the fraction at HPLC retention time of 41.6 minutes.

FIG. 11-7 shows a UV spectrum of any compound from (compound 7) to (compound 10), which was obtained from the fraction at HPLC retention time of 45.1 minutes.

FIG. 11-8 shows a UV spectrum of any compound from (compound 7) to (compound 10), which was obtained from the fraction at HPLC retention time of 45.9 minutes.

FIG. 12 shows an HPLC chart of sample solutions after reaction, which contain (compound 15) or (compound 16).

FIG. 13-1 shows a mass spectrum of (compound 15) or (compound 16), which was obtained from the fraction at HPLC retention time of 38.4 minutes.

FIG. 13-2 shows a mass spectrum of (compound 15) or (compound 16), which was obtained from the fraction at HPLC retention time of 39.2 minutes.

FIG. 13-3 shows a mass spectrum of (compound 15) or (compound 16), which was obtained from the fraction at HPLC retention time of 39.8 minutes.

FIG. 13-4 shows a mass spectrum of (compound 15) or (compound 16), which was obtained from the fraction at HPLC retention time of 40.4 minutes.

FIG. 14-1 shows a UV spectrum of (compound 15) or (compound 16), which was obtained from the fraction at HPLC retention time of 38.4 minutes.

FIG. 14-2 shows a UV spectrum of (compound 15) or (compound 16), which was obtained from the fraction at HPLC retention time of 39.2 minutes.

FIG. 14-3 shows a UV spectrum of (compound 15) or (compound 16), which was obtained from the fraction at HPLC retention time of 39.8 minutes.

FIG. 14-4 shows a UV spectrum of (compound 15) or (compound 16), which was obtained from the fraction at HPLC retention time of 40.4 minutes.

FIG. 15 shows an HPLC chart of sample solutions after reaction, which contain (compound 17) or (compound 18) and (compound 19) or (compound 20).

FIG. 16-1 shows a mass spectrum of (compound 17) or (compound 18) and (compound 19) or (compound 20), which was obtained from the fraction at HPLC retention time of 19.0 minutes.

FIG. 16-2 shows a mass spectrum of (compound 17) or (compound 18) and (compound 19) or (compound 20), which was obtained from the fraction at HPLC retention time of 20.0 minutes.

FIG. 16-3 shows a mass spectrum of (compound 17) or (compound 18) and (compound 19) or (compound 20), which was obtained from the fraction at HPLC retention time of 33.3 minutes.

FIG. 16-4 shows a mass spectrum of (compound 17) or (compound 18) and (compound 19) or (compound 20), which was obtained from the fraction at HPLC retention time of 34.4 minutes.

FIG. 17-1 shows a UV spectrum of (compound 17) or (compound 18) and (compound 19) or (compound 20), which was obtained from the fraction at HPLC retention time of 19.0 minutes.

FIG. 17-2 shows a UV spectrum of (compound 17) or (compound 18) and (compound 19) or (compound 20), which was obtained from the fraction at HPLC retention time of 20.0 minutes.

FIG. 17-3 shows a UV spectrum of (compound 17) or (compound 18) and (compound 19) or (compound 20), which was obtained from the fraction at HPLC retention time of 33.3 minutes.

FIG. 17-4 shows a UV spectrum of (compound 17) or (compound 18) and (compound 19) or (compound 20), which was obtained from the fraction at HPLC retention time of 34.4 minutes.

FIG. 18 shows an HPLC chart of sample solutions after reaction, which contain (compound 21) or (compound 22).

FIG. 19-1 shows a mass spectrum of (compound 21) or (compound 22), which was obtained from the fraction at HPLC retention time of 38.2 minutes.

FIG. 19-2 shows a mass spectrum of (compound 21) or (compound 22), which was obtained from the fraction at HPLC retention time of 39.1 minutes.

FIG. 20-1 shows a UV spectrum of (compound 21) or (compound 22), which was obtained from the fraction at HPLC retention time of 38.2 minutes.

FIG. 20-2 shows a UV spectrum of (compound 21) or (compound 22), which was obtained from the fraction at HPLC retention time of 39.1 minutes.

FIG. 21 shows an HPLC chart of sample solutions after reaction, which contain (compound 23) or (compound 24).

FIG. 22-1 shows a mass spectrum of (compound 23) or (compound 24), which was obtained from the fraction at HPLC retention time of 43.0 minutes.

FIG. 22-2 shows a mass spectrum of (compound 23) or (compound 24), which was obtained from the fraction at HPLC retention time of 43.4 minutes.

FIG. 23-1 shows a UV spectrum of (compound 23) or (compound 24), which was obtained from the fraction at HPLC retention time of 43.0 minutes.

FIG. 23-2 shows a UV spectrum of (compound 23) or (compound 24), which was obtained from the fraction at HPLC retention time of 43.4 minutes.

FIG. 24 shows an HPLC chart of sample solutions after reaction, which contain (compound 25) or (compound 26).

FIG. 25-1 shows a mass spectrum of (compound 25) or (compound 26), which was obtained from the fraction at HPLC retention time of 42.7 minutes.

FIG. 25-2 shows a mass spectrum of (compound 25) or (compound 26), which was obtained from the fraction at HPLC retention time of 43.2 minutes.

FIG. 26-1 shows a UV spectrum of (compound 25) or (compound 26), which was obtained from the fraction at HPLC retention time of 42.7 minutes.

FIG. 26-2 shows a UV spectrum of (compound 25) or (compound 26), which was obtained from the fraction at HPLC retention time of 43.2 minutes.

FIG. 27 shows an HPLC chart of sample solutions after reaction, which contain (compound 27) or (compound 28).

FIG. 28-1 shows a mass spectrum of (compound 27) or (compound 28), which was obtained from the fraction at HPLC retention time of 44.5 minutes.

FIG. 28-2 shows a mass spectrum of (compound 27) or (compound 28), which was obtained from the fraction at HPLC retention time of 45.0 minutes.

FIG. 29-1 shows a UV spectrum of (compound 27) or (compound 28), which was obtained from the fraction at HPLC retention time of 44.5 minutes.

FIG. 29-2 shows a UV spectrum of (compound 27) or (compound 28), which was obtained from the fraction at HPLC retention time of 45.0 minutes.

FIG. 30 shows an HPLC chart of sample solutions after reaction, which contain (compound 29) or (compound 30).

FIG. 31-1 shows a mass spectrum of (compound 29) or (compound 30), which was obtained from the fraction at HPLC retention time of 52.4 minutes.

FIG. 31-2 shows a mass spectrum of (compound 29) or (compound 30), which was obtained from the fraction at HPLC retention time of 54.4 minutes.

FIG. 32-1 shows a UV spectrum of (compound 29) or (compound 30), which was obtained from the fraction at HPLC retention time of 52.4 minutes.

FIG. 32-2 shows a UV spectrum of (compound 29) or (compound 30), which was obtained from the fraction at HPLC retention time of 54.4 minutes.

FIG. 33 shows an HPLC chart of sample solutions after reaction, which contain (compound 31) or (compound 32).

FIG. 34-1 shows a mass spectrum of (compound 31) or (compound 32), which was obtained from the fraction at HPLC retention time of 30.7 minutes.

FIG. 34-2 shows a mass spectrum of (compound 31) or (compound 32), which was obtained from the fraction at HPLC retention time of 32.1 minutes.

FIG. 35-1 shows a UV spectrum of (compound 31) or (compound 32), which was obtained from the fraction at HPLC retention time of 30.7 minutes.

FIG. 35-2 shows a UV spectrum of (compound 31) or (compound 32), which was obtained from the fraction at HPLC retention time of 32.1 minutes.

FIG. 36 shows an HPLC chart of a sample solution after reaction of luteolin and caffeic acid.

FIG. 37 shows an HPLC chart of a sample solution after reaction of apigenin and caffeic acid.

FIG. 38-1 shows an HPLC chart of sample solutions after reaction, which contain a compound group from (compound 33) to (compound 36) and a compound group from (compound 37) to (compound 40).

FIG. 38-2 shows a chromatogram of a compound group from (compound 37) to (compound 40) in m/z817 selective ion detection mode.

FIG. 38-3 shows a chromatogram of a compound group from (compound 33) to (compound 36) in m/z1117 selective ion detection mode.

FIG. 39-1 shows a UV spectrum of a compound group from (compound 37) to (compound 40), which was obtained from the fraction at HPLC retention time of 44.6 minutes.

FIG. 39-2 shows a UV spectrum of a compound group from (compound 33) to (compound 36), which was obtained from the fraction at HPLC retention time of 53.3 minutes.

### DETAILED DESCRIPTION OF THE PREFERRED EMBODIMENTS

Preferred as the “alkoxy group” is a straight chain or branched chain alkoxy group having 1 to 4 carbon atoms and examples thereof include methoxy, ethoxy, propoxy, isopropoxy, butoxy, t-butoxy and the like.

The “sugar residue consisting of 1 or 2 sugars selected from the group consisting of glucose, rhamnose, fructose and galactose” is, for example,

a monosaccharide residue consisting of glucose, rhamnose, fructose or galactose;
a sugar residue consisting of oligosaccharide wherein 2 sugars selected from the group consisting of glucose, rhamnose, fructose and galactose are bonded via a glycoside bond; and the like.

Here, the “sugar residue” is an embodiment of bonding to a hydroxyl group of the flavonol skeleton via a glycoside bond.

Examples of the “oligosaccharide” of the “sugar residue consisting of oligosaccharide wherein 2 sugars selected from the group consisting of glucose, rhamnose, fructose and galactose are bonded via a glycoside bond” include maltose, trehalose, isotrehalose, kojibiose, sophorose, nigerose, laminaribiose, cellobiose, isomaltose, gentiobiose, lactose, rutinose, 2-O-(α-L-rhamnopyranosyl)-D-glucopyranose and the like.

Specific examples of the “sugar residue consisting of 1 or 2 sugars selected from the group consisting of glucose, rhamnose, fructose and galactose” include a glucopyranosyloxy group, a rhamnopyranosyloxy group, a galactopyranosyloxy group, a 4-O-(α-D-glucopyranosyl)-D-glucopyranosyloxy group, a 2-O-(α-L-rhamnopyranosyl)-D-glucopyranosyloxy group and the like.

The “flavonol compound” is a compound having a flavone skeleton having a hydroxyl group at the 3-position and, for example, a compound represented by the formula (III)

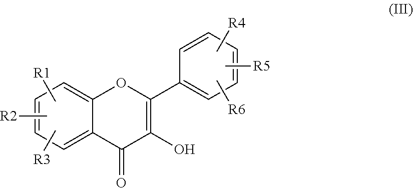

wherein R1, R2, R3, R4, R5 and R6 are as defined above. Specific examples thereof include quercetin, isorhamnetin, kaempferol, tamarixetin, kaempferide, kaempferol-7-neohesperidin and the like.

The “chlorogenic acid” is an ester compound wherein one caffeic acid (the above-mentioned formula IV-a) is ester-bonded to quinic acid and, for example, a compound represented by the above-mentioned formula (IV-c) and the like. Preferable examples of the chlorogenic acid include a compound represented by the formula:

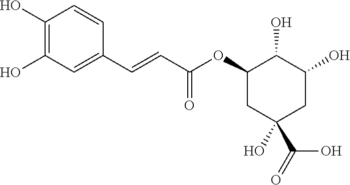

and the like.

The “dicaffeoylquinic acid” is a diester compound wherein two caffeic acids (the above-mentioned formula IV-a) are ester-bonded to quinic acid and, for example, a compound represented by the above-mentioned formula (IV-d) and the like. Preferable examples of the dicaffeoylquinic acid include a compound represented by the formula:

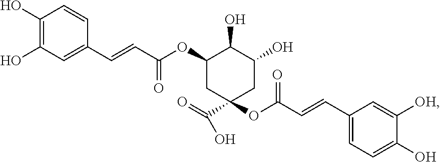

a compound represented by the formula:

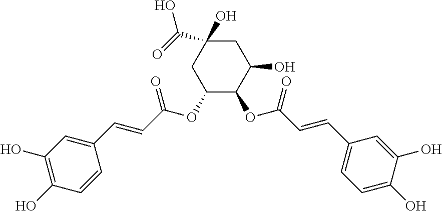

and the like.

Specific examples of the compound of the embodiment of the present invention include the compounds from (compound 1) to (compound 40) described in the following Synthesis Examples. In view of the lipase inhibitory activity, (compound 1) and (compound 2) described in Synthesis Example 1, (compound 3) and (compound 4) described in Synthesis Example 2, (compound 5) and (compound 6) described in Synthesis Example 3, (compound 15) and (compound 16) described in Synthesis Example 5, and (compound 33), (compound 34), (compound 35), (compound 36), (compound 37), (compound 38), (compound 39) and (compound 40) described in Synthesis Example 13 are preferable. More preferred from the aspect of activity are (compound 1) and (compound 2) described in Synthesis Example 1, (compound 5) and (compound 6) described in Synthesis Example 3, (compound 15) and (compound 16) described in Synthesis Example 5, and (compound 33), (compound 34), (compound 35) and (compound 36) described in Synthesis Example 13.

A further embodiment of the present invention is a resultant product obtained by reacting one or more kinds of components selected from the group consisting of caffeic acid, gallic acid, chlorogenic acid, dicaffeoylquinic acid, catechin, gallocatechin, catechin gallate and gallocatechin gallate with one or more kinds of flavonol compounds in the presence of polyphenoloxydase. The resultant product may be a mixture with an unreacted product and a byproduct.

Specific examples of the resultant product of the present invention include resultant products obtained by reacting one or more kinds of caffeic acid, gallic acid, chlorogenic acid, catechin and dicaffeoylquinic acid with one or more kinds selected from quercetin, isorhamnetin, kaempferol, tamarixetin, kaempferide and kaempferol-7-neohesperidin together with polyphenoloxydase. In view of the lipase inhibitory activity, a resultant product obtained by reacting one or more kinds selected from caffeic acid, gallic acid, chlorogenic acid, catechin and dicaffeoylquinic acid with quercetin together with polyphenoloxydase is preferable. More preferred from the aspect of activity is a resultant product obtained by reacting one or more kinds selected from caffeic acid, chlorogenic acid, catechin and dicaffeoylquinic acid with quercetin together with polyphenoloxydase.

The flavonol compound, and caffeic acid, gallic acid, chlorogenic acid, dicaffeoylquinic acid, catechin, gallocatechin, catechin gallate and gallocatechin gallate, which is the starting material of the above-mentioned compounds, may be commercially available products.

Alternatively, the flavonol compound can also be obtained by extraction from natural materials such as onion, green tea, apple, buckwheat, fagopyrum tataricum, broccoli, spinach, kale, parsley, pine leaf, red wine, grape, ginkgo leaf, nettle and the like.

In addition, gallic acid can also be obtained by extraction from natural materials such as burdock, lotus root, Geranium thunbergii, raspberry, chestnut coat, guarana, guava, laurel, pomegranate, Crataegus, camellia, Tussilago and the like.

Moreover, caffeic acid, chlorogenic acid and dicaffeoylquinic acid can also be obtained by extraction from natural materials such as coffee bean, artemisia, stem and leaf of sweet potato, garland chrysanthemum, fiddlehead, udo, Petasites, Parasenecio delphiniifolius, Siberuan ginseng, Cynara scolymus, echinacea and the like.

Furthermore, catechin, gallocatechin, catechin gallate and gallocatechin gallate can also be obtained by extraction from natural materials such as green tea, oolong tea, cocoa, pine bark, grape seed and the like.

Examples of the polyphenoloxydase to be used in the present invention include laccase, bilirubinoxydase, peroxidase, ascorbic acid oxidase, ceruloplasmin and the like. Of these, laccase is preferable from the aspect of yield. Laccase is known to widely present in plants, animals and microorganisms, and plant-derived and microorganism-derived laccases are preferable. Examples of the microorganism-derived laccase include those derived from bacteria and fungi (including filamentous bacterium and yeast). Specific examples include those derived from Aspergillus; Neurospora; Pyricularia such as Pyricularia orizae (P. pryzae) and the like; Trametes such as Trametes villosa (T. villosa), Trametes versicolor (T. versicolor) and the like; Rhizoctonia such as Rhizoctonia solani (R. solani) and the like; Coprinus such as Coprinus cinereus (C. cinereus) and the like; Coriolus such as Coriolus hirsutus (C. hirsutus), Coriolus versicolor (C. versicolor) and the like. In addition, examples of the commercially available laccase include “Laccase DAIWA Y120” (Amano Enzyme Inc.) and the like. Furthermore, polyphenoloxydases prepared from fruit, vegetable, mushroom, microorganism and the like can also be used. These laccases may be used alone or two or more kinds thereof may be used in combination.

The oxidation polymerization reaction is performed using a combination of one or more kinds of flavonol compounds represented by the formula (III)

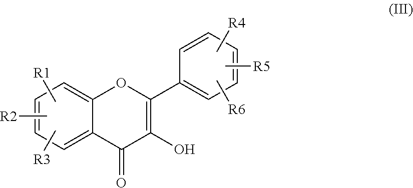

wherein R1, R2, R3, R4, R5 and R6 are as defined in the above-mentioned (1),
and one or more kinds of components of a compound selected from the group consisting of
a compound represented by the following structural formula,

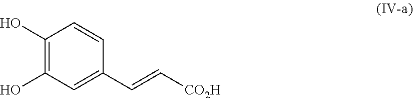

(i.e., caffeic acid), a compound represented by

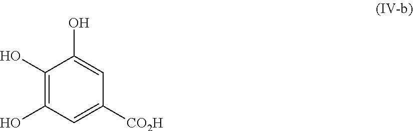

(i.e., gallic acid), a compound represented by

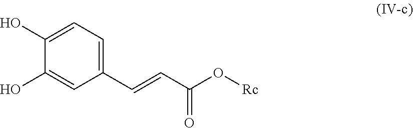

in the formula (IV-c),
Rc is a group represented by the following formula

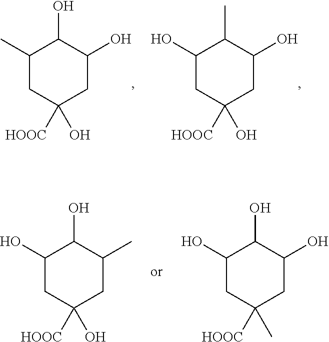

(i.e., chlorogenic acid), a compound represented by

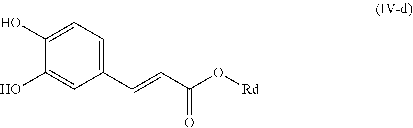

in the formula (IV-d),
Rd is a group represented by the following formula

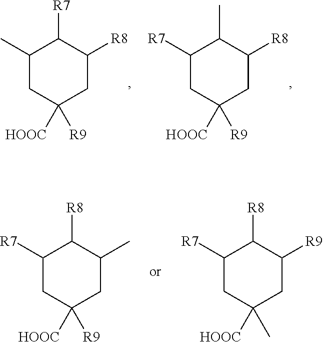

wherein
any one group of R7, R8 and R9 is a group represented by the formula:

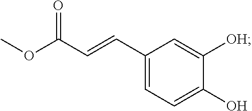

and
two groups of the remaining R7, R8 and R9 are hydroxyl groups (i.e., dicaffeoylquinic acid), a compound represented by

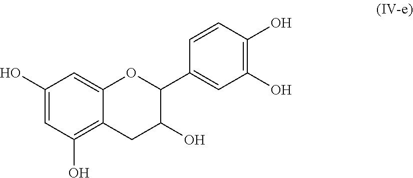

(i.e., catechin), a compound represented by

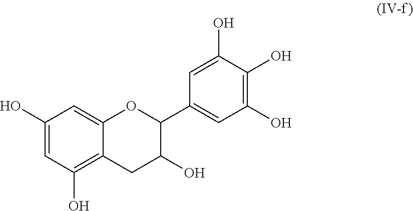

(i.e., gallocatechin), a compound represented by

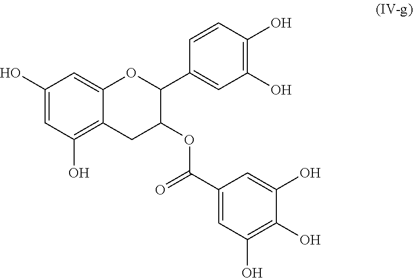

(i.e., catechin gallate), and a compound represented by

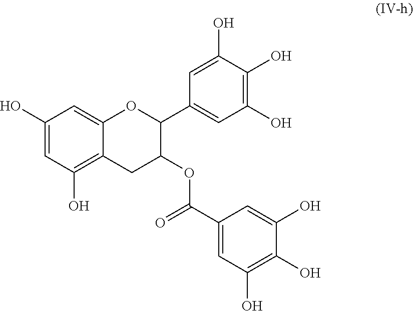

(i.e., gallocatechin gallate), in the presence of polyphenoloxydase in an aqueous solution at pH 3 to 7, preferably pH 4 to 6, at 20 to 80° C., preferably 40 to 60° C., for 1 minute to 60 minutes, preferably 1 minute to 30 minutes. The amount of Laccase DAIWA Y120, which is polyphenoloxydase, to be used is 1 mg to 100 mg, preferably 10 mg to 50 mg, per 100 mg of the starting material. The oxidation polymerization reaction can be terminated by deactivating the enzyme by adding an equivalent to 2-fold amount, preferably equivalent amount, of an organic solvent, preferably ethanol, relative to the reaction mixture.

Thereafter, when, where necessary, the resultant product obtained by the polymerization reaction is purified or an isomer having a different reaction site is separated, the separation can be performed by chromatography using a reversed-phase resin wherein carbon chains having 1 to 30 carbon atoms are bonded, a normal phase resin using silica gel as a carrier, a polystyrene type synthetic adsorbent (Diaion HP-20, HP-21, Sepabeads SP825, SP850, SP70, SP700), a polystyrene type synthetic adsorbent (Sepabeads SP207), or a methacrylic synthetic adsorbent (Diaion HP1MG, HP2MG). As an elution solvent to be used for the above-mentioned chromatographys, water, as well as lower alcohols such as methanol, ethanol, propanol, isopropanol and the like, polyhydroxylic alcohols such as 1,3-butanediol, propanediol, dipropanediol, glycerol and the like, ethers such as diethyl ether, dipropyl ether and the like, esters such as ethyl acetate, butyl acetate and the like, ketones such as acetone, ethylmethylketone and the like, organic solvents such as chloroform, dichloromethane, acetonitrile, hexane and the like can be used, and one or more kinds selected therefrom are used. In addition, saline, phosphate buffer, phosphoric acid-buffered saline and the like may be used. Furthermore, pH may be adjusted by adding trifluoroacetic acid and the like.

Alternatively, the resultant product obtained by the oxidation polymerization can also be produced by similarly treating a combination of a natural material containing one or more kinds of flavonol compounds represented by the formula (III)

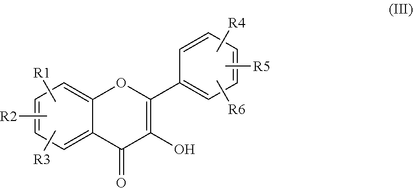

wherein R1, R2, R3, R4, R5 and R6 are as defined in the above-mentioned (1),
and a natural material containing one or more kinds of components of a compound selected from the group consisting of a compound represented by the following structural formula,

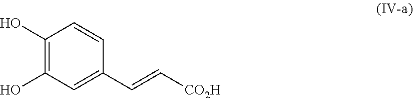

(i.e., caffeic acid), a compound represented by

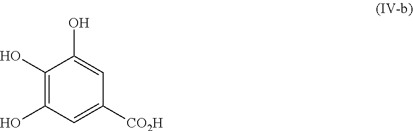

(i.e., gallic acid), a compound represented by

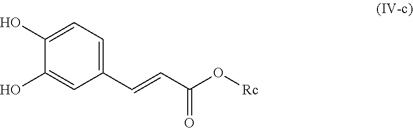

in the formula (IV-c),
Rc is a group represented by the following formula

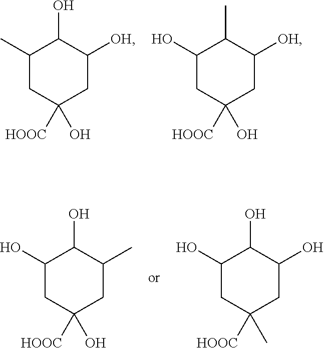

(i.e., chlorogenic acid), a compound represented by

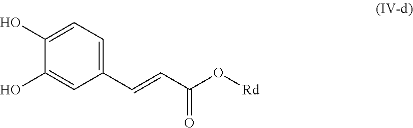

in the formula (IV-d),
Rd is a group represented by the following formula

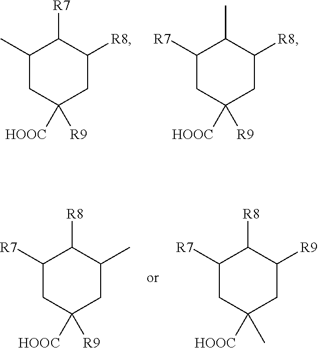

wherein
any one group of R7, R8 and R9 is a group represented by the formula:

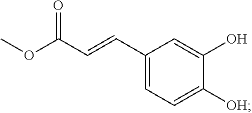

and
two groups of the remaining R7, R8 and R9 are hydroxyl groups (i.e., dicaffeoylquinic acid), a compound represented by

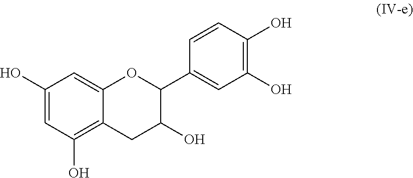

(i.e., catechin), a compound represented by

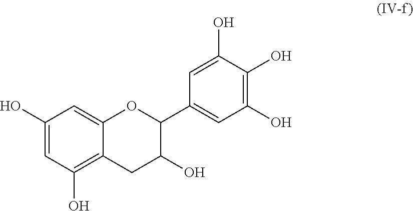

(i.e., gallocatechin), a compound represented by

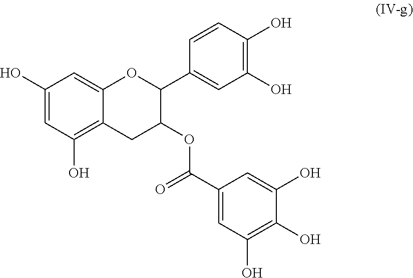

(i.e., catechin gallate), and a compound represented by

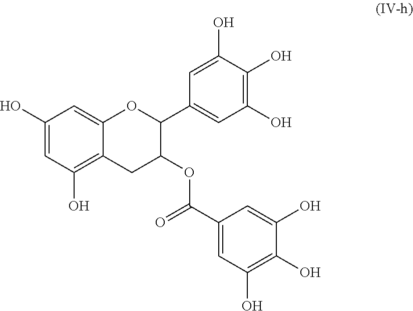

(i.e., gallocatechin gallate) in the presence of polyphenoloxydase. The reaction can be performed under conditions similar to those of the above-mentioned oxidation polymerization reaction.

As the above-mentioned natural materials, plant (e.g., onion, green tea, apple, buckwheat, fagopyrum tataricum, broccoli, spinach, kale, parsley, pine leaf, red wine, grape, ginkgo leaf, nettle etc.) and the like can be used. The natural material may be disrupted or ground, added with water and used as an aqueous solution, or the natural material may also be extracted by the following method, and the extract may be added with water and used as an aqueous solution. While the extraction method may vary depending on the natural material, the following method is generally used for the preparation. That is, while the natural material in a raw state may be subjected to extraction, extraction is preferably performed after treatments such as chopping, drying, pulverization and the like in consideration of the extraction efficiency. Extraction is performed by immersing the material in an extraction solvent. Stirring may be performed and homogenization in an extraction solvent may also be performed to increase extraction efficiency. The extraction temperature may be room temperature or under heating, suitably about 1° C. to not more than the boiling point of the extraction solvent, generally 1° C. to 100° C., and preferably 20° C. to 90° C. While the extraction time varies depending on the natural material to be the extraction target, the kind of the extraction solvent, and extraction temperature, it is suitably about 4 hours to 14 days.

As the extraction solvent, water, as well as lower alcohols such as methanol, ethanol, propanol, isopropanol and the like, polyhydroxylic alcohols such as 1,3-butanediol, propanediol, dipropanediol, glycerol and the like, ethers such as diethyl ether, dipropyl ether and the like, esters such as ethyl acetate, butyl acetate and the like, ketones such as acetone, ethylmethylketone and the like, and organic solvents such as chloroform, dichloromethane, acetonitrile, hexane and the like can be used, and one or more kinds selected therefrom are used. In addition, saline, phosphate buffer, phosphoric acid-buffered saline and the like may also be used.

Among the above-mentioned extraction solvents, water, organic solvent, and a mixed solvent thereof can be preferably used in the present invention. As the organic solvent, lower alcohol, 1,3-butanediol, glycerol, ethers, ethyl acetate, acetone, chloroform, dichloromethane, acetonitrile and hexane can be preferably used, and one or more kinds selected therefrom are used. As the lower alcohols, methanol and ethanol are particularly preferable and, as the ether, diethyl ether is particularly preferable.

Since the compound or resultant product of the present invention has a superior lipase inhibitory action and low toxicity, it can be used for the prophylaxis or improvement of a condition related to lipase.

“Inhibiting lipase” means to eliminate or attenuate the activity by hydrolyzing the ester bond of triacylglycerol, which is the main component of fat, or specifically inhibiting the function of lipase as an enzyme. For example, it means to specifically inhibit the function of lipase based on the conditions of the below-mentioned physiological activity Test Examples.

Since the compound or resultant product of the present invention has a superior lipase inhibitory action, a condition related to lipase activity in a subject (e.g., mammal, preferably human, mouse, rat, hamster, rabbit, cat, dog, bovine, sheep, monkey) can be prevented or improved by inhibiting lipase in the subject by ingestion of the compound or resultant product of the present invention by the subject.

While the amount of ingestion of one or more kinds selected from the compound and the resultant product of the present invention varies depending on the condition and age of the subject who ingests, preparation method, ingestion form, ingestion route and the like of an extract, it is about 0.01 g to 20 g/day, preferably about 0.05 g to 10 g/day, more preferably about 0.1 g to 3 g/day, for a human adult. The amount can be ingested at once, or in 2 to 5 portions as necessary.

While the compound or resultant product obtained by the present invention can be used as it is, it may also be used after dissolving a concentrated or dried product again in water or an organic solvent, or after a purification treatment such as decoloring, deodorizing, desalting and the like without impairing the lipase inhibitory action, or after a fractionation treatment by column chromatography using an ion exchange resin and the like. For preservation, moreover, the compound or resultant product may be freeze-dried after a purification treatment and used by dissolving in a solvent when in use. In the present invention, an extract of the above-mentioned plant by using the above-mentioned solvent or the aforementioned treated product is directly used, or added to an aqueous carrier such as water, lower alcohol and the like, a base such as emulsion, gel, cream and the like, or powderized or granulated to give a composition. In addition, it may be included in a vesicle such as liposome and the like, a microcapsule and the like.

Furthermore, additives such as cellulose (crystalline cellulose, hydroxypropylcellulose etc.) and a derivative thereof, starches (wheat starch, cornstarch, sodium carboxymethyl starch, dextrin etc.) and a derivative thereof, natural polymer compounds (gum arabic, sodium alginate etc.), sugars (glucose, maltose, sorbitol, maltitol, mannitol etc.) and a derivative thereof, excipients such as inorganic salts (sodium chloride, calcium carbonate, magnesium silicate etc.) and the like, binders (guar gum, synthetic aluminum silicate, stearic acid, polymer polyvinylpyrrolidone, lactose etc.), lubricants (talc, magnesium stearate, polyethylene glycol 6000 etc.), disintegrants (adipic acid, calcium stearate, sucrose etc.), surfactants (sucrose ester of fatty acid, soybean lecithin, polyoxyethylene hydrogenated castor oil, polyoxyethylenemonostearic acid ester and the like), thickeners (sodium carboxymethylcellulose, carboxyvinyl polymer, xanthan gum, gelatin etc.), coating agents (ethyl acrylate•methyl methacrylate copolymer dispersion, caramel, Carnauba wax, shellac, sucrose, pullulan etc.), pH adjusters (citric acid, sodium citrate, acetic acid, sodium acetate, sodium hydroxide etc.), antioxidants (ascorbic acid, tocopherol acetate, natural vitamin E, propyl gallate etc.), flavoring agents (aspartame, licorice extract, saccharin etc.), preservatives (sodium benzoate, sodium edetate, sorbic acid, sodium sorbate, methyl p-hydroxybenzoate, butyl p-hydroxybenzoate etc.), colorants (ferric oxide red, iron oxide yellow, iron oxide black, carnine, Food Color Blue No. 1, Food Color yellow No. 4, Food Color yellow No. 4 Aluminum Lake, Food Color Red No. 2, sodium copper chlorophyllin etc.) can be added.

Since the compound or resultant product of the present invention is superior in the safety in oral ingestion, it can be applied by a convenient method of oral ingestion, or a method such as external use and the like. In the present invention, one or more kinds selected from the compound and the resultant product may be directly ingested, or may be ingested together with various additives and the like. Furthermore, it may be formulated using various carriers, bases, additives and the like, and can be ingested in the form of a tablet including sugar-coated tablet and film-coated agent, pill, capsule, ampoule, syrup, suspension, emulsion, elixir, drop, troche, chewable, powder, granule or the like.

Since the compound or resultant product of the present invention has a superior lipase inhibitory activity, an effect can be expected even when it is ingested as a food or drink.

The “food and drink” of the present invention means food and drink in general, and also includes, besides general foods including so-called health foods, foods for specified health uses and foods with nutrient function claims defined in food system with health claims of Ministry of Health, Labour and Welfare, and further includes foods and drinks for dieting.

Examples of the “foods and drinks for dieting” include dietary supplements and the like.

Examples of the above-mentioned “foods and drinks” include tea drinks, beverage, health foods and the like.

Examples of the “tea drinks” include liquid tea (e.g., liquid green tea, liquid oolong tea, liquid red tea, liquid mixed tea), tea leaves (e.g., green tea leaf, oolong tea leaf, red tea leaf, green tea leaf, mixed tea leaf), powdered tea obtained by drying tea drinks (e.g., powdered green tea, powdered oolong tea, powdered red tea, powdered mixed tea) and the like.

The “beverage” is not particularly limited as long as it is a beverage other than the above-mentioned tea drinks and examples thereof include juice, coffee, cocoa and the like.

The “health foods” are not particularly limited, and examples thereof include health foods known per se. For example, they may be prepared in the forms of tablet, capsule, powder, granule, suspension, chewable, syrup and the like, or may be added to confectionery, jelly, frozen dessert such as ice cream and the like, milk products such as yogurt, milk and the like, and various emulsion foods and drinks such as purine, mousse, Bavarian cream, dressing and the like.

The form of the composition for food or drink of the so present invention is not particularly limited and may be any form as long as it permits oral ingestion. Examples thereof include powder, granule, tablet, hard capsule, soft capsule, liquid (drinks, jelly drinks etc.), candy, chocolate and the like, all of which can be produced according to a method known per se in the pertinent technical field.

While the content of one or more kinds selected from the compound and resultant product of the present invention in the composition for food or drink varies depending on the extraction conditions and form of addition of plant, dosage form, ingestion form and the like, it is generally 0.05 wt % to 100 wt %, preferably 0.5 wt % to 90 wt %, based on the weight of the food or drink. Particularly, it is generally 0.05 wt % to 100 wt %, preferably 0.5 wt % to 90 wt %, as an extract, based on the weight of the food or drink.

The composition for food or drink of the present invention can contain other food additives where necessary. Examples of such food additives include fruit juice, dextrin, cyclic oligosaccharide, saccharides (monosaccharides such as fructose, glucose etc. and polysaccharides), acidulant, flavor, powdered green tea and the like for adjusting and improving taste, emulsifier, collagen, whole milk powder, polysaccharide thickener, agar and the like for improving texture, and further, those being used as components of general health foods and the like such as vitamins, eggshell calcium, calcium pantothenate, other minerals, royal jelly, propolis, honey, dietary fiber, Agaricus, chitin, chitosan, flavonoids, carotenoids, lutein, Chinese herbal medicine, chondroitin, various amino acids and the like.

In addition, the composition can also be added to veterinary feed for mouse, rat, hamster, rabbit, cat, dog, bovine, sheep, monkey and the like.

Furthermore, the compound or resultant product of the present invention can be added to an external preparation, and can be provided as a skin external preparation, perfumery, cosmetics and the like.

The compound or resultant product of the present invention effectively inhibits hydrolysis and absorption of fat, effectively inhibits decomposition of carbohydrates, and further has an antioxidant action. Therefore, the compound or resultant product of the present invention can be used as an active ingredient of various pharmaceutical compositions such as an agent for the prophylaxis or improvement of diseases related to lipase, for example, fat absorption inhibitor, postprandial hyperlipemia and lipid dysbolism, an antiobesity agent, an insulin sensitizer, a blood insulin concentration-improving or -lowering agent, and the like. In addition, since the pharmaceutical composition of the present invention inhibits lipase produced by human skin indigenous bacteria such as Propionibacterium acnes and the like, and suppresses production of lipoperoxide, it can be used for the prophylaxis or improvement of dermatic diseases such as acne and the like and body odor caused by bacterial lipase and peroxidation of lipid.

When the pharmaceutical composition of the present invention is used, the dose of one or more kinds selected from the compound and resultant product obtained by the present invention varies depending on the conditions, disease state and age of administration subject (patient etc.), preparation method of extract, dosage form, administration form, administration route and the like. It is about 0.01 g to 20 g/day, preferably about 0.05 g to 10 g/day, more preferably about 0.1 g to 3 g/day, for a human adult. This can be administered at once or in 2 to 5 portions as necessary.

In addition, compound (I) may be present as a single stereoisomer, a racemic compound, or a mixture of enantiomers and diastereomers. The compound may also be present as a geometric isomer. All such single stereoisomers, racemic compounds and mixtures thereof, and geometric isomers are intended to be within the scope of the present invention.

Other features of the invention will become apparent in the course of the following descriptions of exemplary embodiments which are given for illustration of the invention and are not intended to be limiting thereof.

### EXAMPLES

Synthesis Examples, Synthesis Reference Examples and Physiological Activity Test Examples are described below.

### Synthesis Example 1

### Synthesis Example of Compounds with the Structures Shown Below

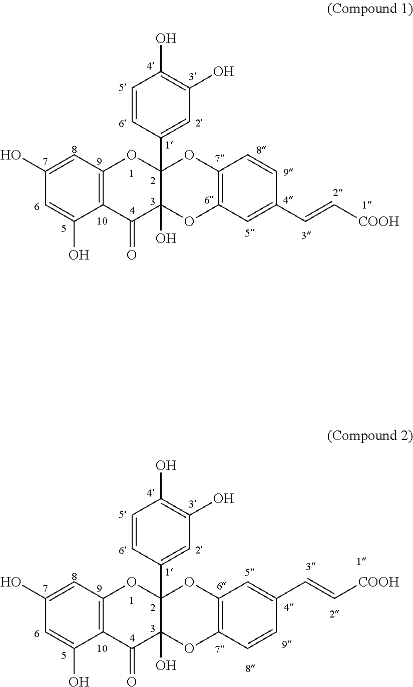

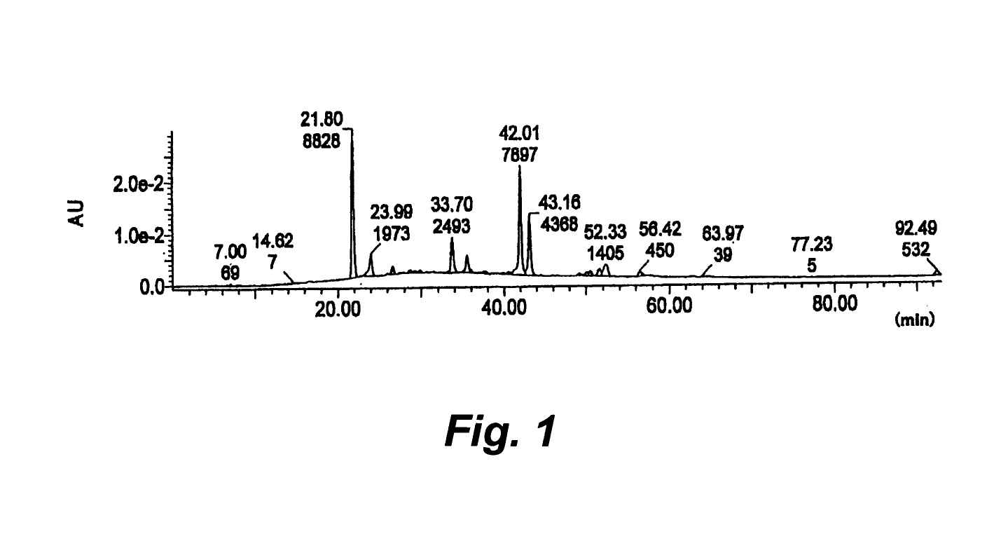

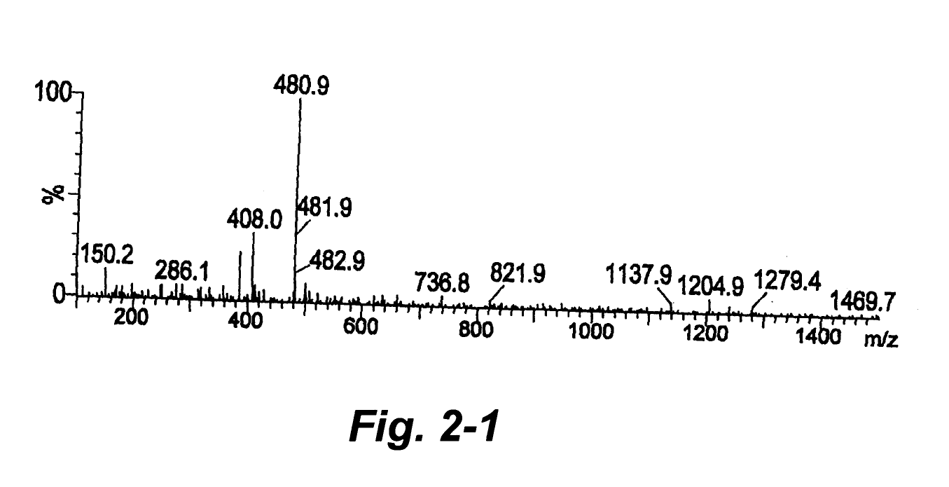

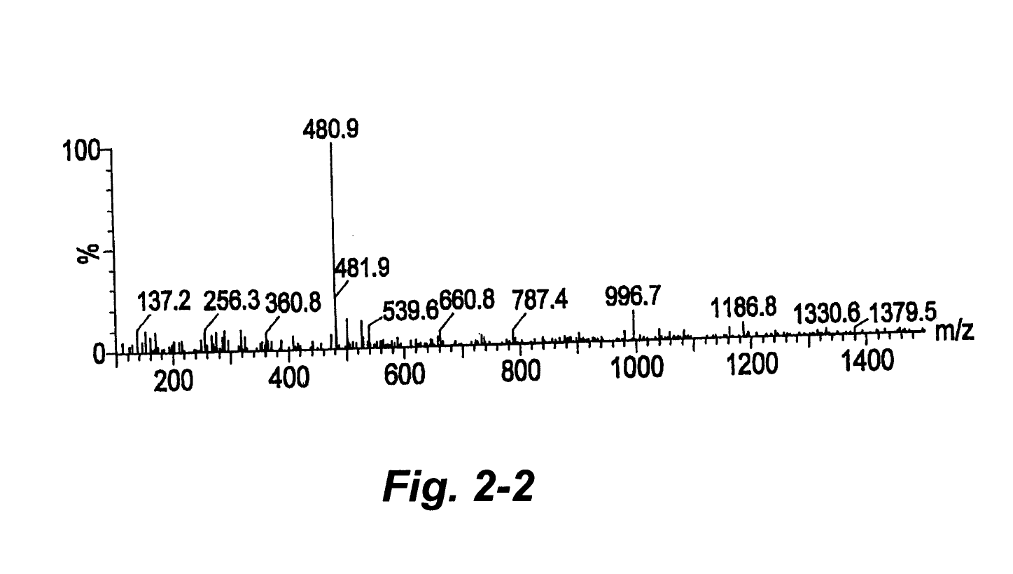

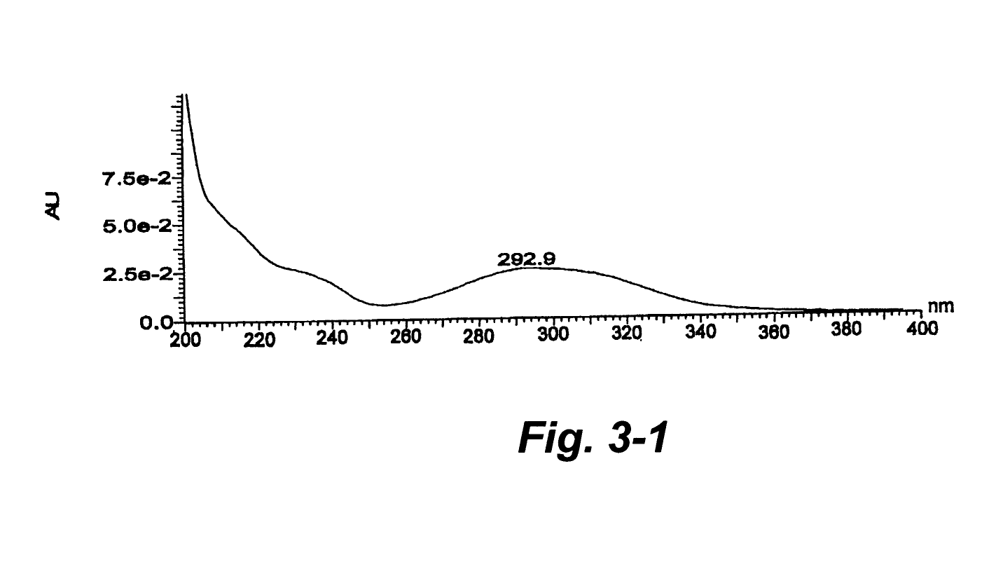

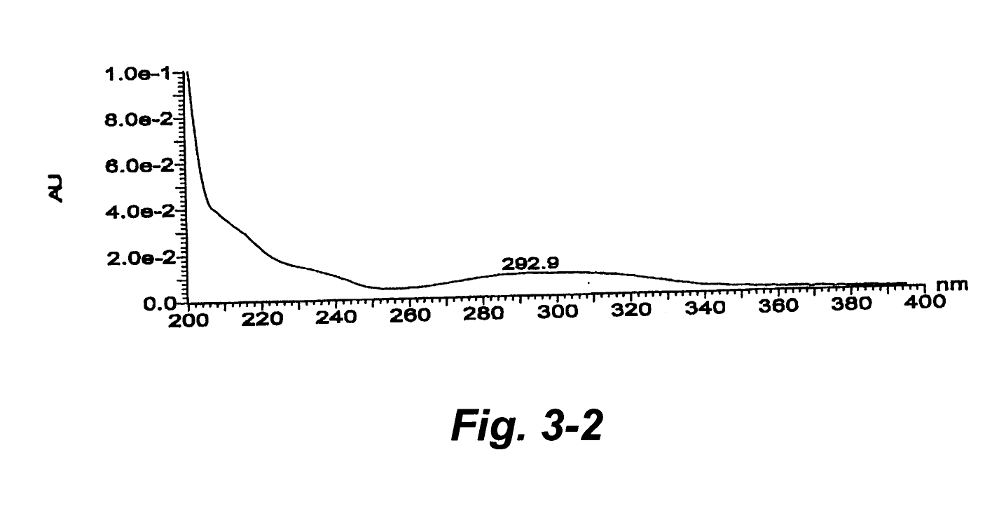

Quercetin 2 hydrate (720 mg) and caffeic acid (270 mg) were dissolved in purified water (1 L), and the mixture was warmed in a water bath at 50° C. for 30 minutes. Laccase DAIWA Y120 (100 mL, Amano Enzyme Inc.) prepared to 5 mg/mL with purified water was added and the mixture was stirred for 1 minute. Ethanol (1 L) was added to quench the enzyme reaction. This reaction mixture was concentrated to dryness under reduced pressure, the sample was dissolved in purified water, and the solution was loaded on Sep-Pak C18 (Waters), washed with purified water, eluted with 30% aqueous ethanol solution (100 mL). 30% Ethanol-eluted fraction was concentrated to dryness under reduced pressure, the fraction was dissolved in 50% aqueous ethanol solution (2 mL), and the solution (500 μL) was injected in 4 portions and purified by HPLC. Two components (12 mg and 7 mg) having a molecular weight of 480 were obtained from the fractions with retention time 42.01 minutes and 43.16 minutes. The apparatus and measurement conditions of HPLC are shown below. LC/MS was used for the detection of the component having a molecular weight of 480 by HPLC, and UV spectrum was simultaneously measured by a photodiode array detector (see FIGS. 1 to 3). The apparatus and measurement conditions of LC/MS and photodiode array detector are shown below.

HPLC apparauts:

pump: Hitachi Lachrom Elite L-2130 (manufactured by Hitachi, Ltd.)

detector: Hitachi Lachrom Elite L-2455 (manufactured by Hitachi, Ltd.)

autoinjector: Hitachi Lachrom Elite L-2200 (manufactured by Hitachi, Ltd.)

fraction collector: ADVANTEC SF-3120 (manufactured by ADVANTEC)

HPLC measurement conditions:

column: CAPCELL PAK AQ S-5 μm, 20×250 mm (manufactured by Shiseido Co., Ltd.)

solvent: solution of 0.05% trifluoroacetic acid-35% acetonitrile in water

flow rate: 6 mL/minute

detection wavelength: 280 nm

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

LC/MS analysis conditions:

column: CAPCELL PAK AQ S-3 μm, 2×250 mm (manufactured by

Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid-10% acetonitrile in water, SOLUTION B solution of 0.05% trifluoroacetic acid-80% acetonitrile in water

¹H-NMR (400 MHz, MeOH-d4): shown in Table 1.

¹³C-NMR (100 MHz, MeOH-d4): shown in Table 2.

As the NMR measurement device, Bruker Avance 400 (400 MHz, Bruker BioSpin KK) was used. The attribution of which carbon to which each proton is bonded was determined on the basis of the measurement by two-dimensional NMR (HSQC), and the assumed whole structure was attributed by two-dimensional NMR (HMBC). From the results of NMR analysis of each component, the structural difference of each component was assumed to be (compound 1) or (compound 2) in a flat plane structure; however, (compound 1) and (compound 2) cannot be distinguished by NMR analysis.

### Synthesis Example 2

### Synthesis Example of Compounds with the Structures Shown Below

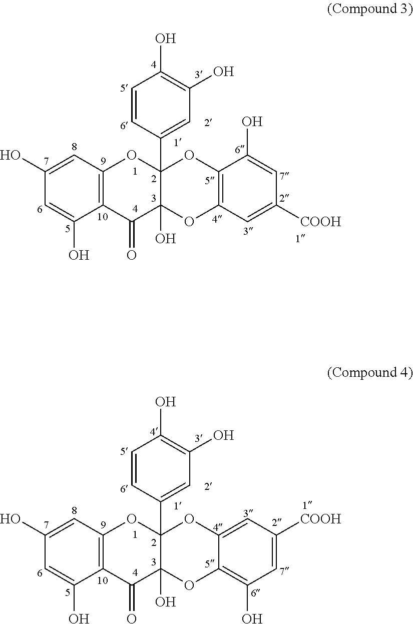

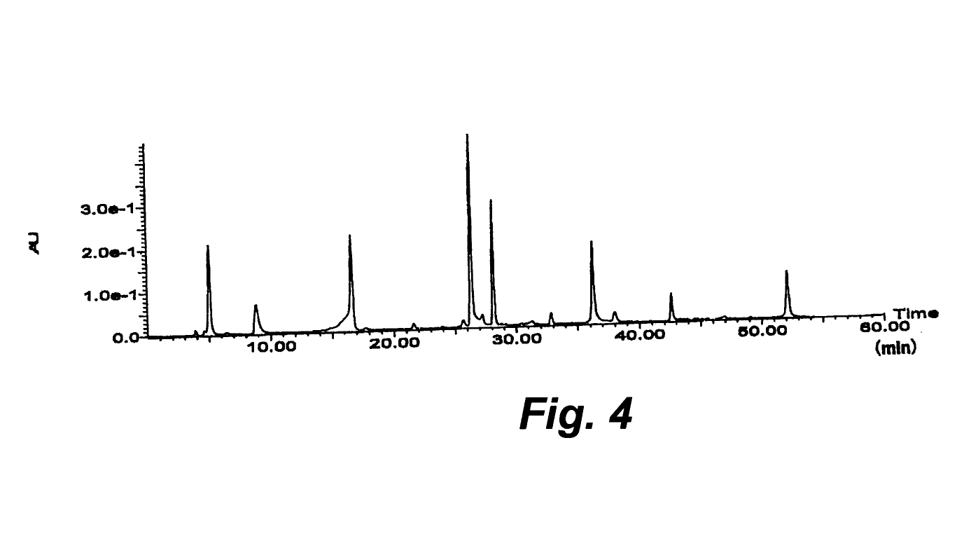

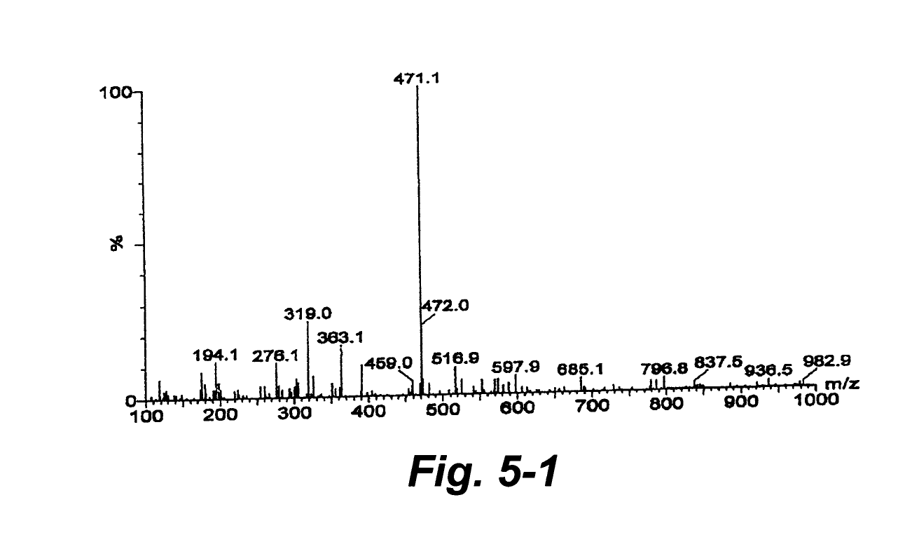

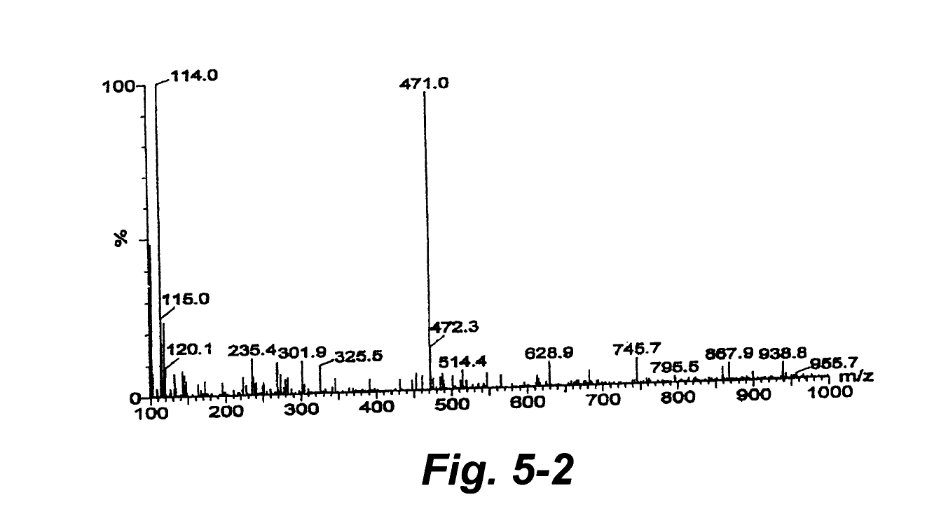

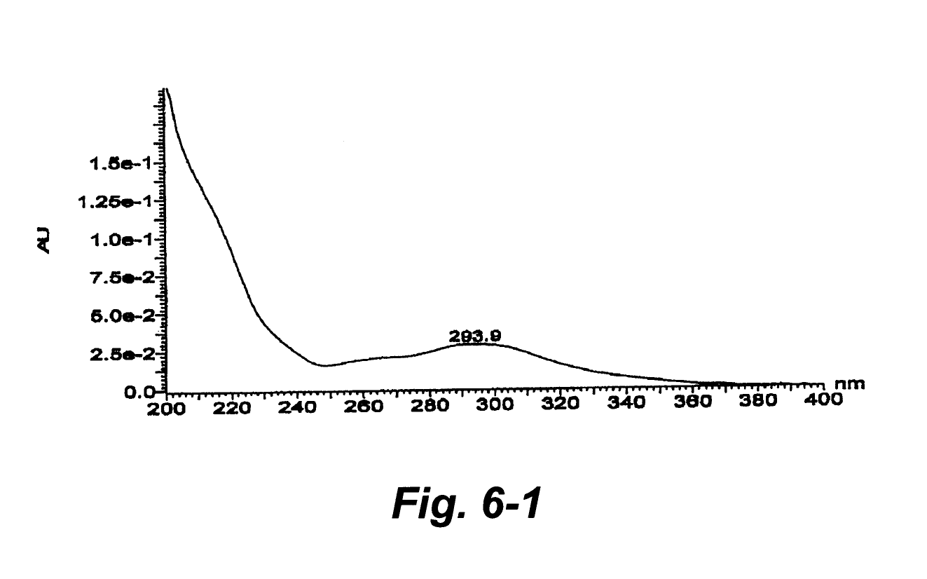

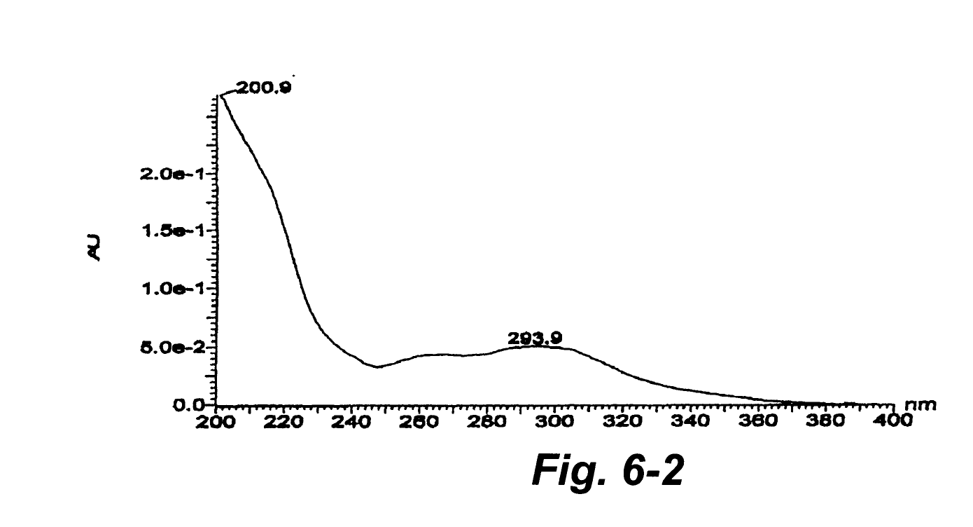

Quercetin 2 hydrate (720 mg) and gallic acid (300 mg) were dissolved in purified water (1 L), and the mixture was warmed in a water bath at 50° C. for 30 minutes. Laccase DAIWA Y120 (100 mL, Amano Enzyme Inc.) prepared to 5 mg/mL was added and the mixture was stirred for 1 minute. Ethanol (1 L) was further added to quench the enzyme reaction. This reaction mixture was concentrated to dryness under reduced pressure, the sample was dissolved in purified water, and the solution was loaded on Sep-Pak C18 (Waters), washed with purified water, and eluted with 30% aqueous ethanol solution (100 mL). 30% Ethanol-eluted fraction was concentrated to dryness under reduced pressure, the fraction was dissolved in 50% aqueous ethanol solution (2 mL), and the solution (500 μL) was injected in 4 portions and purified by HPLC. One component (75 mg) having a molecular weight of 470 was obtained from the fractions with retention time 27.2 minutes and 28.3 minutes. The apparatus and measurement conditions of HPLC are shown below. LC/MS was used for the detection of the component having a molecular weight of 480 by HPLC, and UV spectrum was simultaneously measured by a photodiode array detector (see FIGS. 4 to 6). The apparatus and measurement conditions of LC/MS and photodiode array detector are shown below.

HPLC apparatus:

pump: Hitachi Lachrom Elite L-2130 (manufactured by Hitachi, Ltd.)

detector: Hitachi Lachrom Elite L-2455 (manufactured by Hitachi, Ltd.)

autoinjector: Hitachi Lachrom Elite L-2200 (manufactured by Hitachi, Ltd.)

fraction collector: ADVANTEC SF-3120 (manufactured by ADVANTEC)

HPLC measurement condition:

column: CAPCELL PAK AQ S-5 μm, 20×250 mm (manufactured by Shiseido Co., Ltd.)

solvent: solution of 0.05% trifluoroacetic acid-25% acetonitrile in water

flow rate: 6 mL/minute

detection wavelength: 280 nm

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid-10% acetonitrile in water, SOLUTION B solution of 0.05% trifluoroacetic acid-80% acetonitrile in water

¹H-NMR (400 MHz, MeOH-d4): shown in Table 1.

¹³C-NMR (100 MHz, MeOH-d4): shown in Table 2.

As the NMR measurement device, Bruker Avance 400 (400 MHz, Bruker BioSpin KK) was used. The attribution of which carbon to which each proton is bonded was determined on the basis of the measurement by two-dimensional NMR (HSQC), and the assumed whole structure was attributed by two-dimensional NMR (HMBC). From the results of NMR analysis of each component, the difference was assumed to be any of (compound 3) or (compound 4) in a flat plane structure; however, (compound 3) and (compound 4) cannot be distinguished by NMR analysis.

### Synthesis Example 3

### Synthesis Example of Compounds with the Structures Shown Below

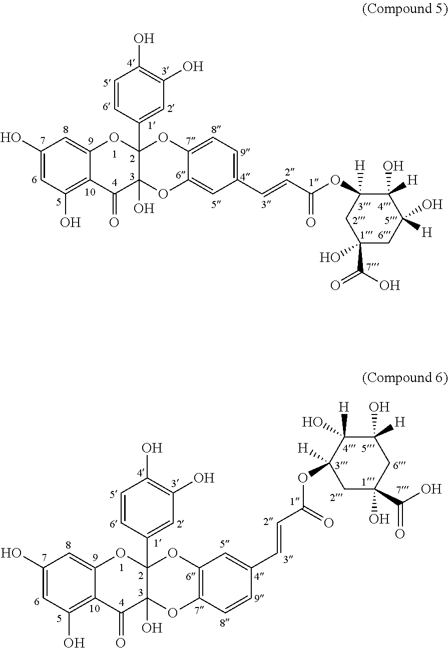

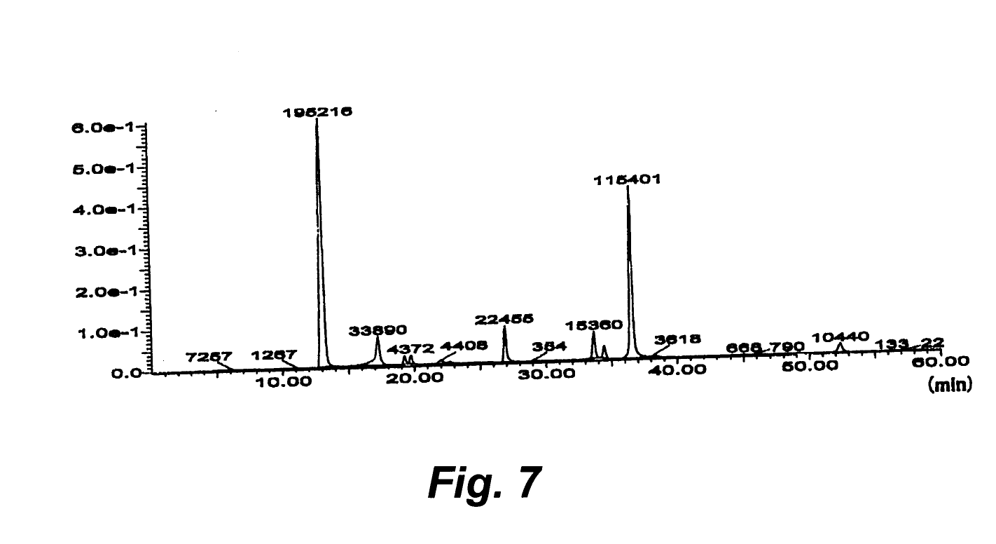

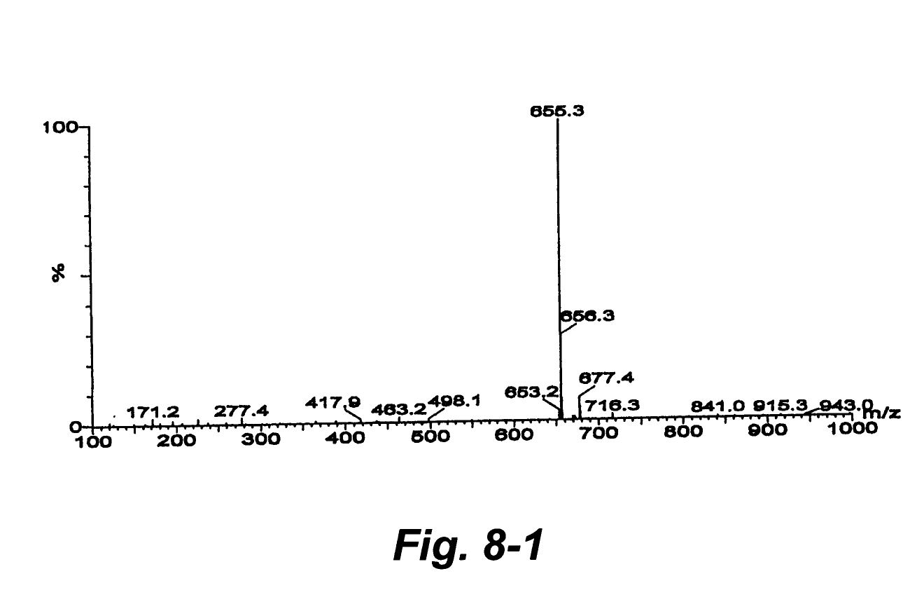

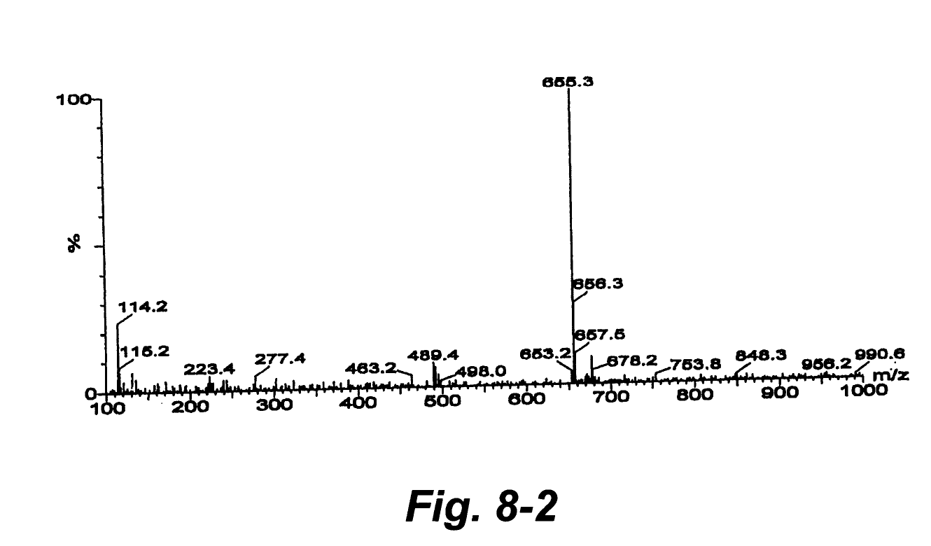

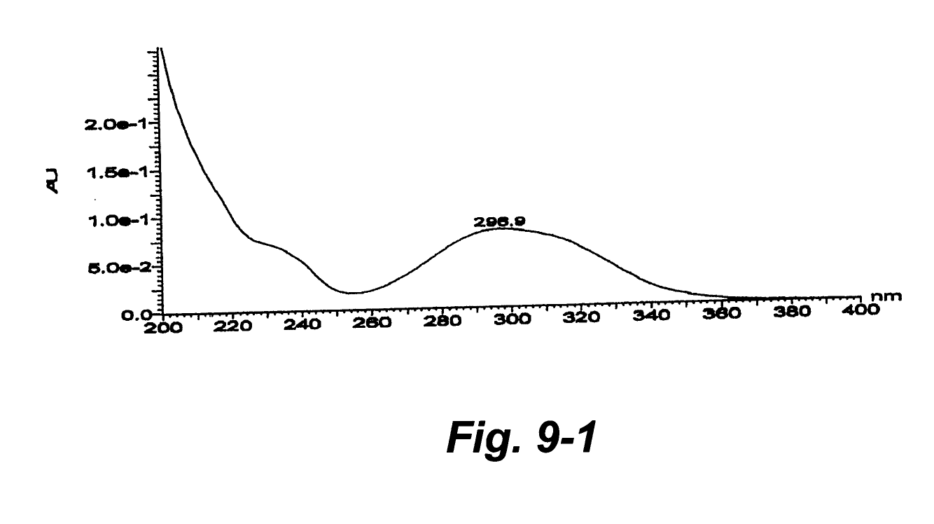

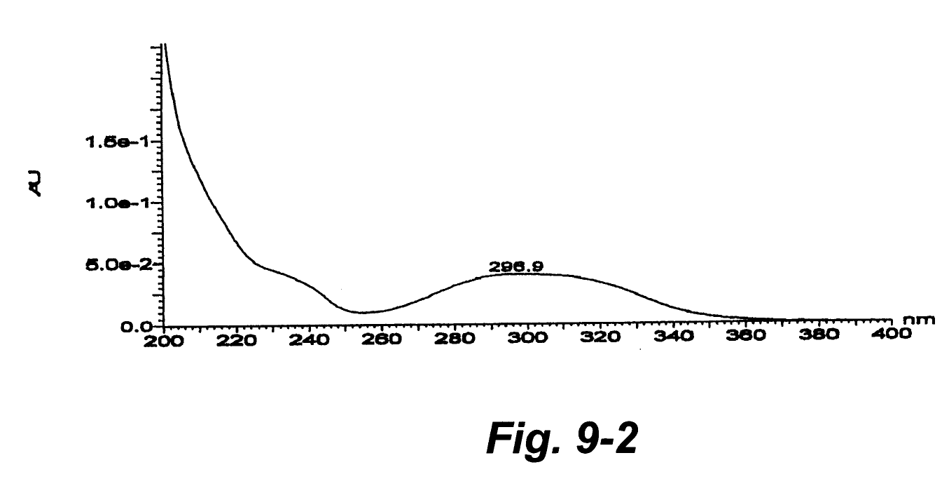

Quercetin 2 hydrate (500 mg) and chlorogenic acid (250 mg) were dissolved in purified water (1000 mL), and the mixture was warmed in an incubator at 50° C. for 1 minute. Laccase DAIWA Y120 (100 mL, Amano Enzyme Inc.) prepared to 5 mg/mL was added and the mixture was stirred for 1 minute. Ethanol (1000 mL) was further added to quench the enzyme reaction. This reaction mixture was concentrated to dryness under reduced pressure, the sample was dissolved in purified water, and the solution was loaded on Sep-Pak C18 (Waters), washed with purified water, and eluted with 30% aqueous ethanol solution (100 mL). 40% Ethanol-eluted fraction was concentrated to dryness under reduced pressure, the fraction was dissolved in 50% aqueous ethanol solution (2 mL), and the solution (500 μL) was injected in 4 portions and purified by HPLC. Two components (10 mg and 3 mg) having a molecular weight of 654 were obtained from the fractions with retention time 33.7 minutes and 34.4 minutes. The apparatus and measurement conditions of HPLC are shown below. LC/MS was used for the detection of the component having a molecular weight of 654 by HPLC, and UV spectrum was simultaneously measured by a photodiode array detector (see FIGS. 7 to 9). The apparatus and measurement conditions of LC/MS and photodiode array detector are shown below.

HPLC apparatus:

pump: PU-2087 PLUS (manufactured by JASCO Corporation) detector: Hitachi Lachrom Elite L-2455 (manufactured by Hitachi, Ltd.)

autoinjector: AS-2057 PLUS (manufactured by JASCO Corporation) fraction collector: ADVANTEC SF-3120 (manufactured by ADVANTEC)

HPLC measurement condition:

column: CAPCELL PAK AQ S-5 μm, 50×250 mm (manufactured by Shiseido Co., Ltd.)

solvent: solution of 0.05% trifluoroacetic acid and 25% acetonitrile in water

flow rate: 30 mL/minute

detection wavelength: 280 nm

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid-10% acetonitrile in water, SOLUTION B solution of 0.05% trifluoroacetic acid and 80% acetonitrile in water

¹H-NMR (400 MHz, MeOH-d4): shown in Table 1.

¹³C-NMR (100 MHz, MeOH-d4): shown in Table 2.

As the NMR measurement device, Bruker Avance 400 (400 MHz, Bruker BioSpin KK) was used. The attribution of which carbon to which each proton is bonded was determined on the basis of the measurement by two-dimensional NMR (HSQC), and the assumed whole structure was attributed by two-dimensional NMR (HMBC). From the results of NMR analysis of each component, the difference was assumed to be any of (compound 5) or (compound 6) in a flat plane structure; however, (compound 5) and (compound 6) cannot be distinguished by NMR analysis.

### Synthesis Example 4

### Synthesis Example of Compounds with the Structures Shown Below

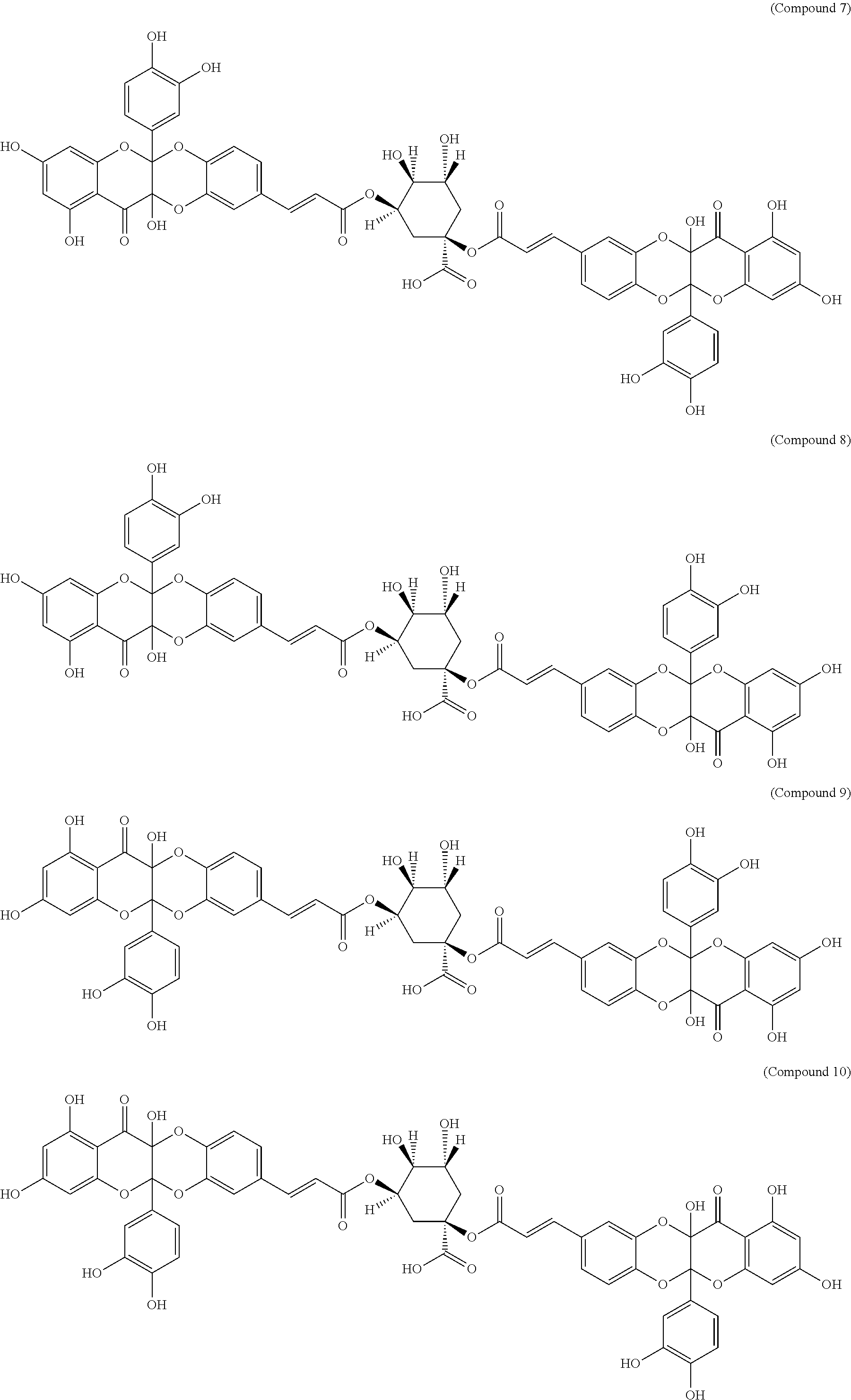

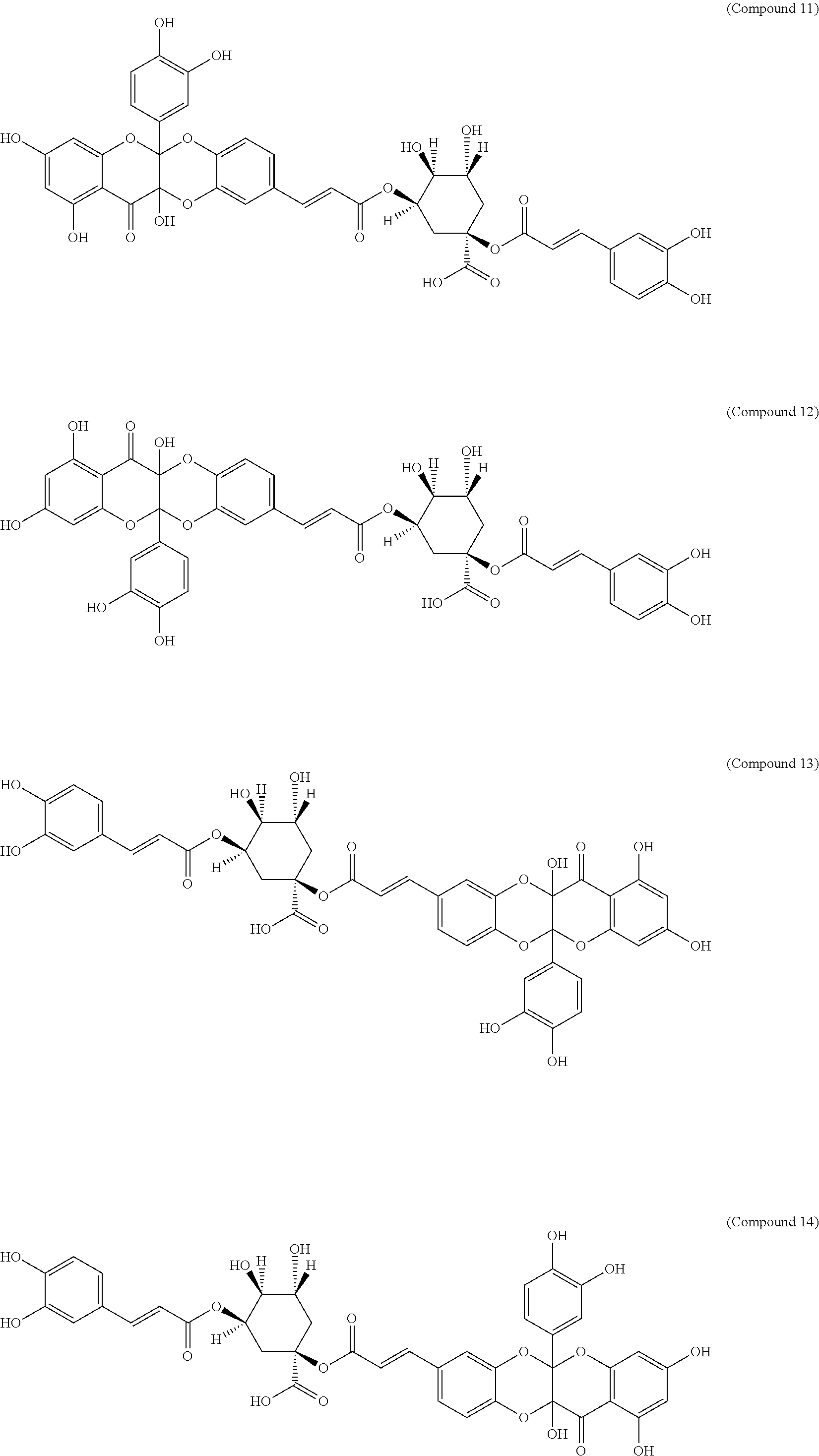

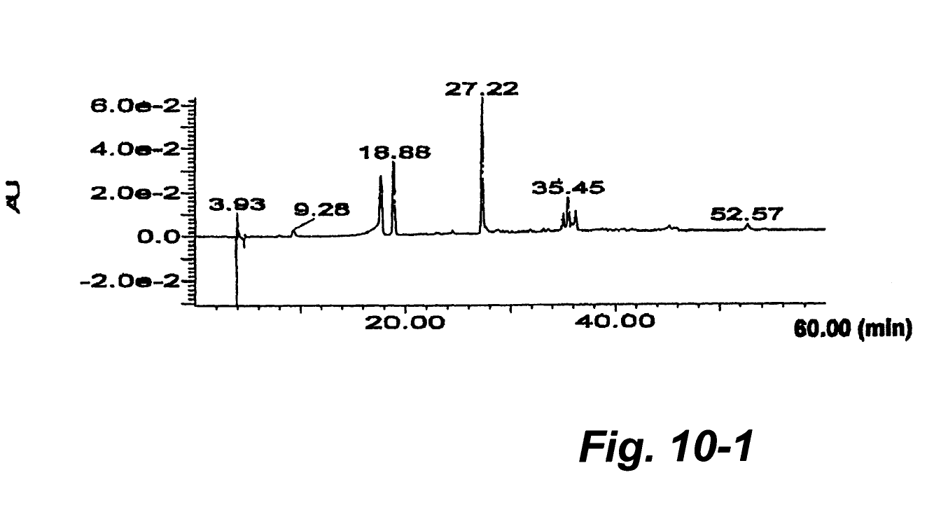

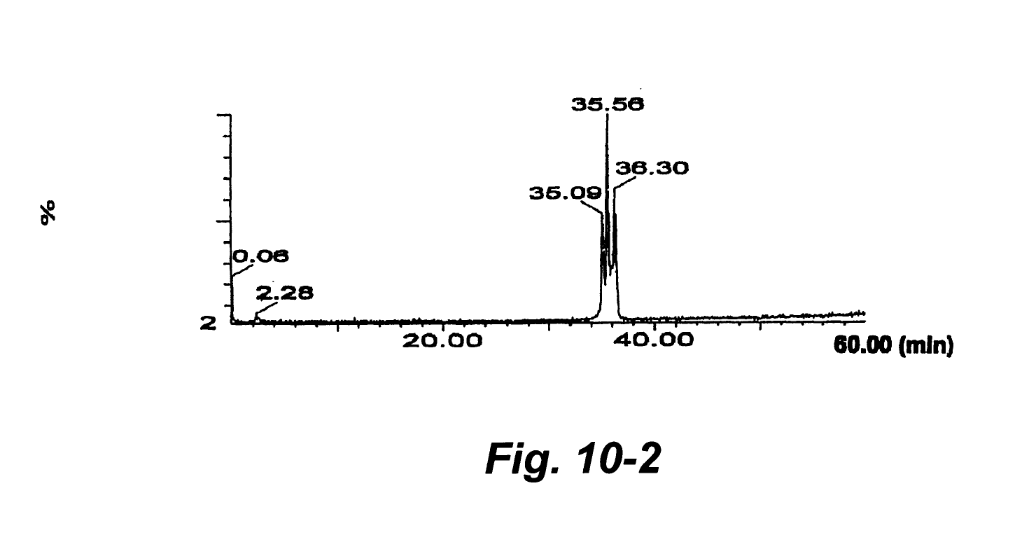

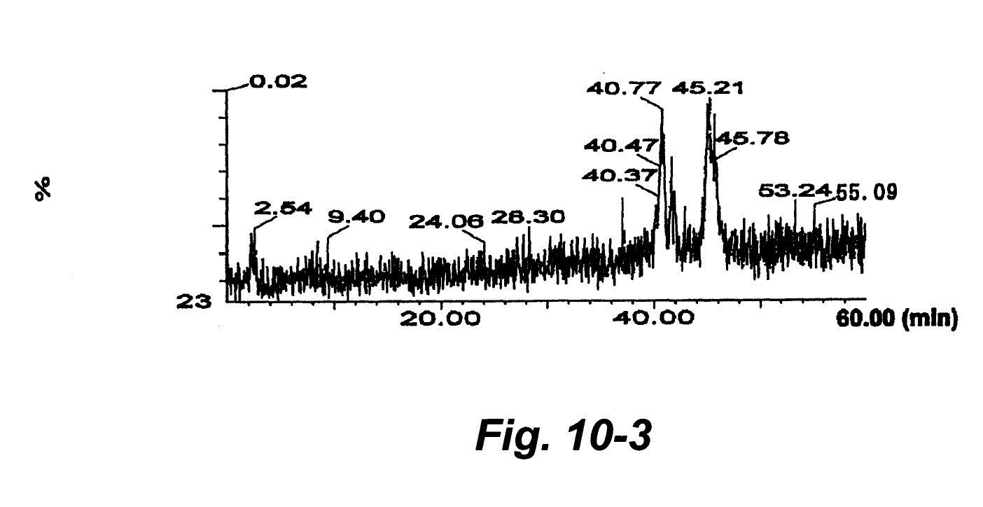

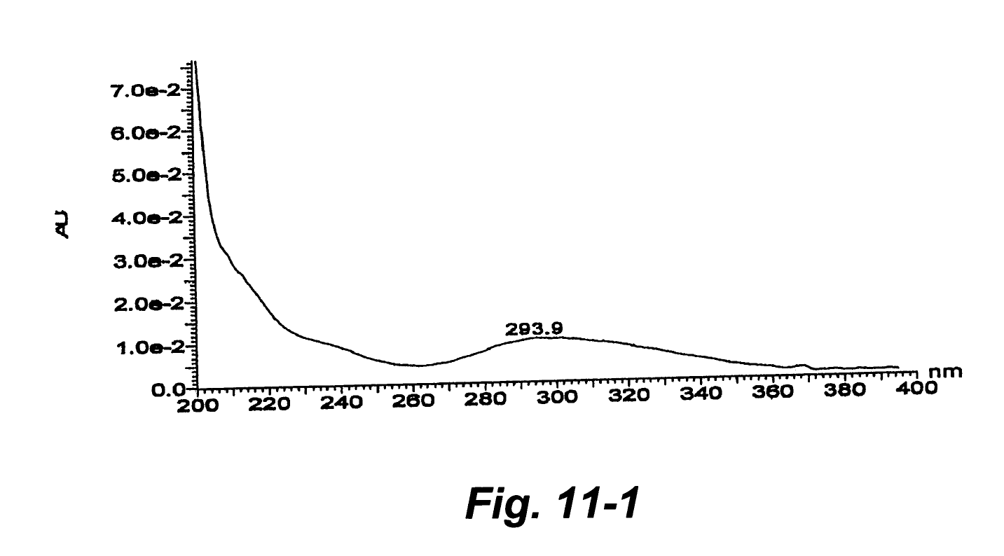

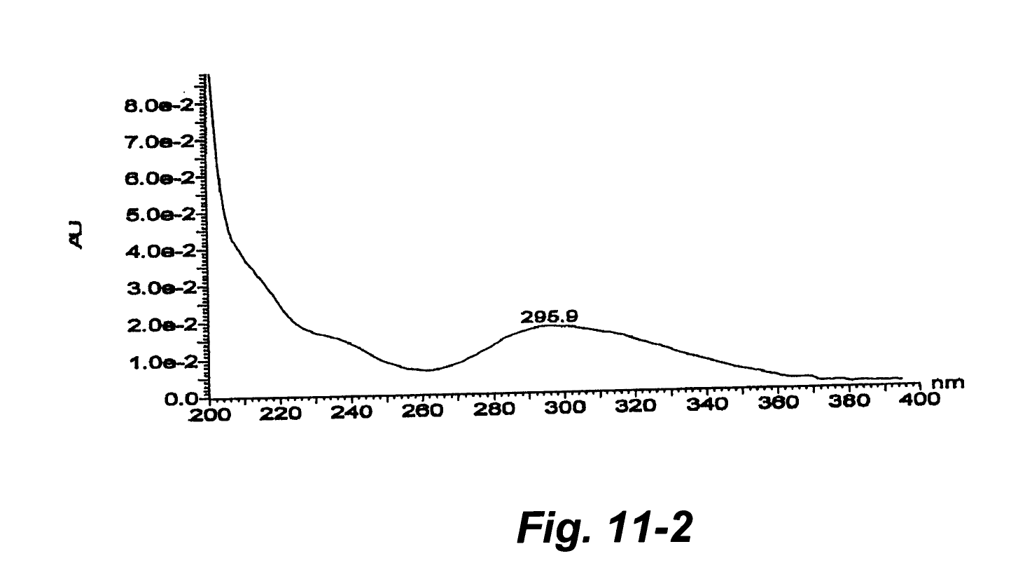

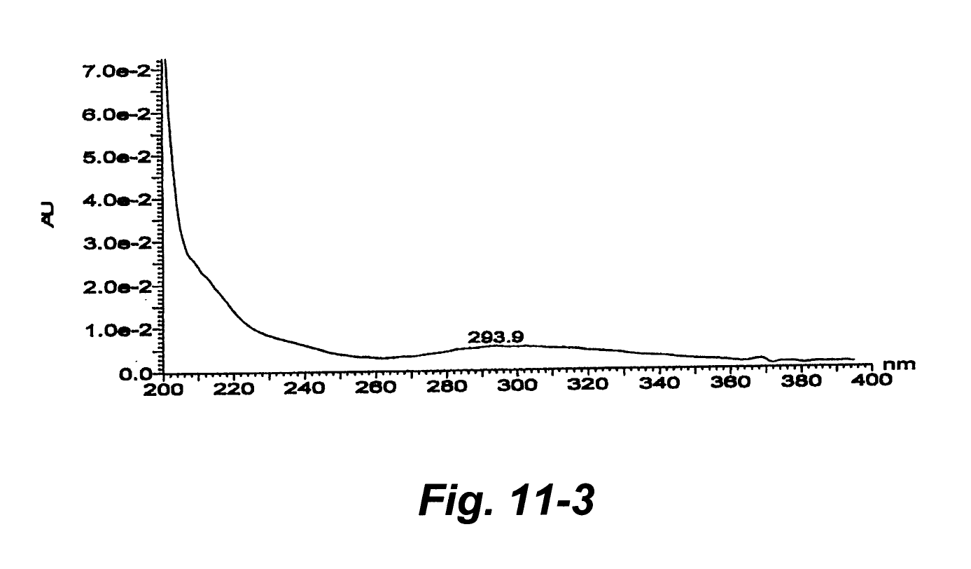

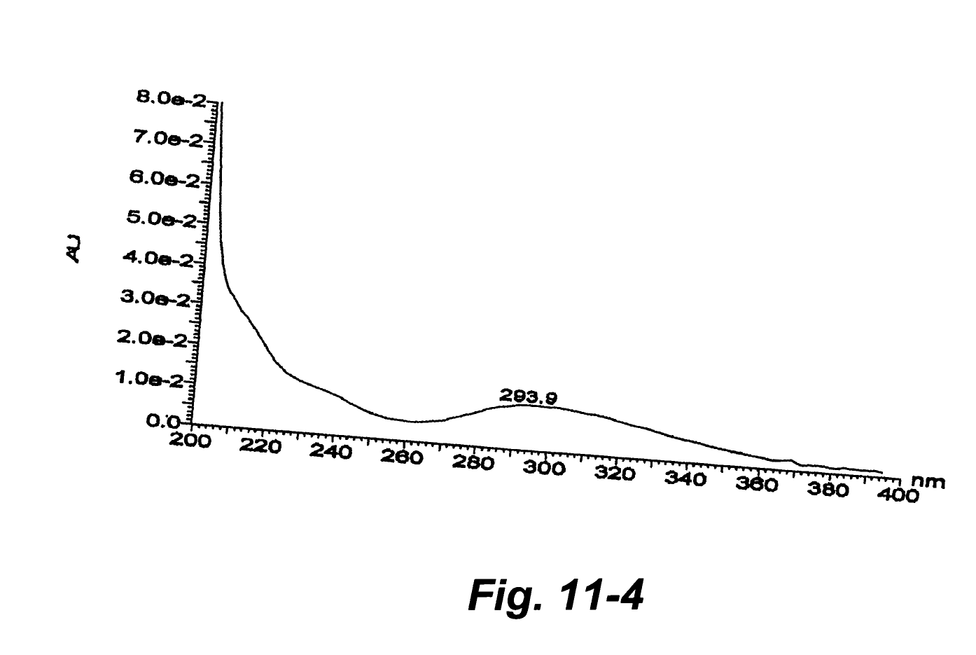

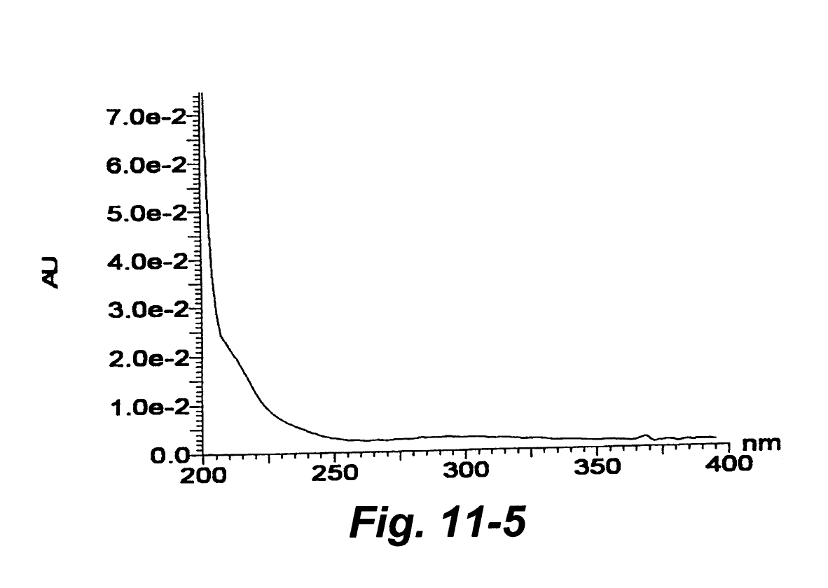

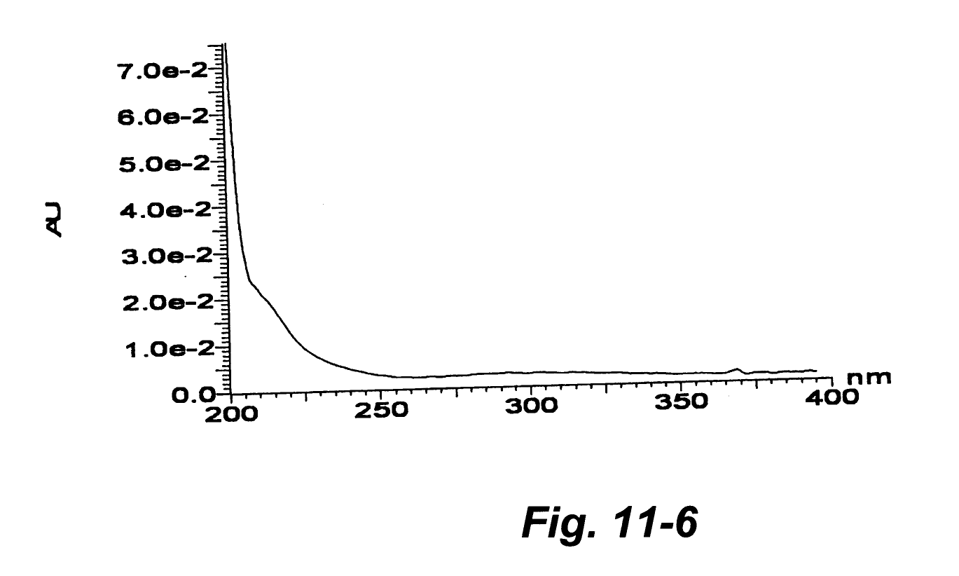

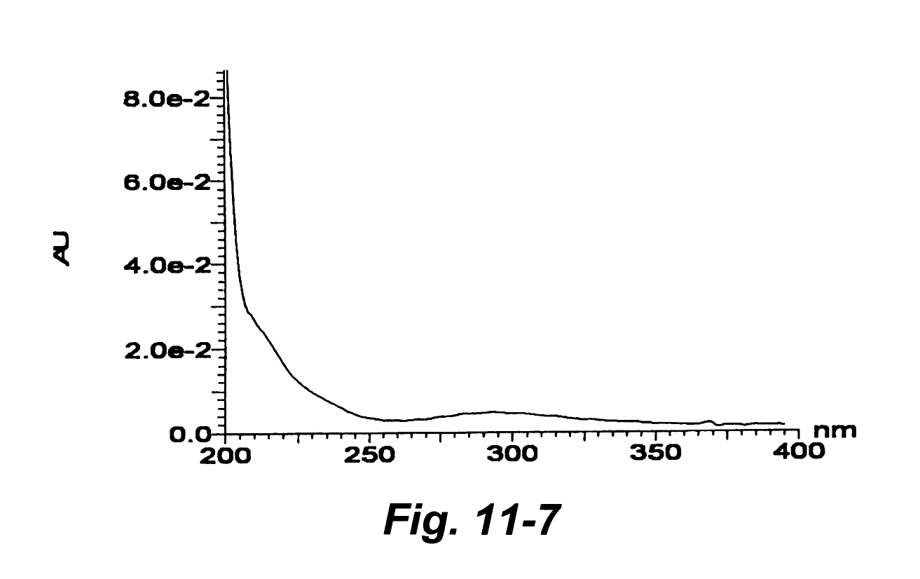

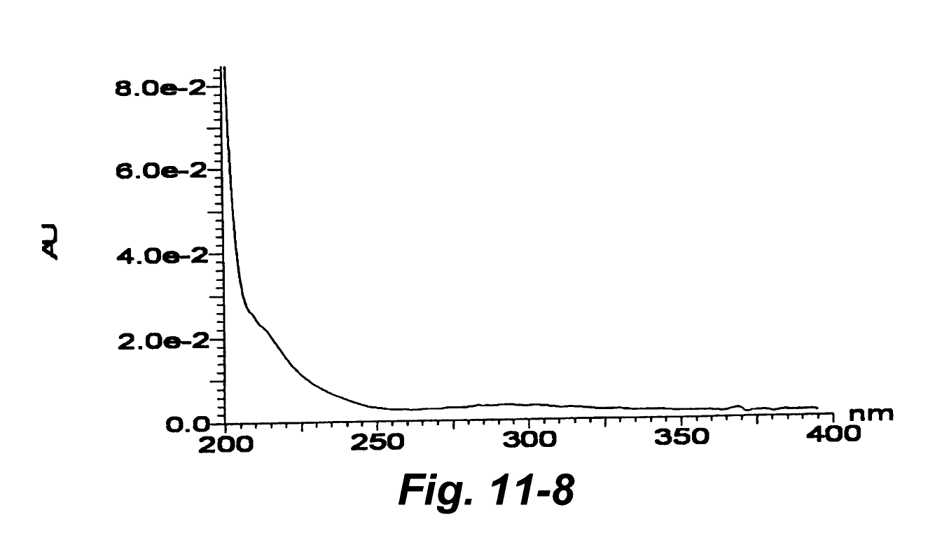

Quercetin 2 hydrate (100 μg) and 1,3-dicaffeoylquinic acid (100 μg) were dissolved in purified water (200 μL), and the mixture was warmed in an incubator at 50° C. for 1 minute. Laccase DAIWA Y120 (10 μL, Amano Enzyme Inc.) prepared to 5 mg/mL was added and the mixture was stirred for 1 minute. Ethanol (200 μL) was further added to quench the enzyme reaction. This reaction mixture (1 μL) was analyzed by LC/MS in selective ion analysis mode, and 4 components having a molecular weight of 816 could be confirmed in the fractions with retention time 35.0 minutes, 35.5 minutes, 35.9 minutes, and 36.2 minutes. From the results of the UV spectrum analysis of each component simultaneously measured by a photodiode array detector (detection wavelength: 300 nm), the absorption around 370 nm derived from ring C of quercetin disappeared and only the absorption around 280 nm derived from ring A and ring B was strongly detected. The above confirms that the structures from (compound 11) to (compound 14) were produced (see FIG. 10 and FIG. 11).

In addition, from the results of the analysis by LC/MS in selective ion analysis mode, a component having a molecular weight of 1116 could be confirmed in the fractions with retention time 40.8 minutes, 41.6 minutes, 45.1 minutes, and 45.9 minutes. Moreover, from the results of the UV spectrum analysis of each component, the absorption around 370 nm derived from ring C of quercetin disappeared, and only the absorption around 280 nm derived from ring A and ring B was strongly detected. The above confirms that the structures from (compound 7) to (compound 10) were produced (see FIG. 10). The apparatus and measurement conditions of LC/MS and photodiode array detector are shown below.

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

m/z817, m/z1117 selective detection mode

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid-10% acetonitrile in water, SOLUTION B solution of 0.05% trifluoroacetic acid-80% acetonitrile in water

### Synthesis Example 5

### Synthesis Example of Compounds with the Structures Shown Below

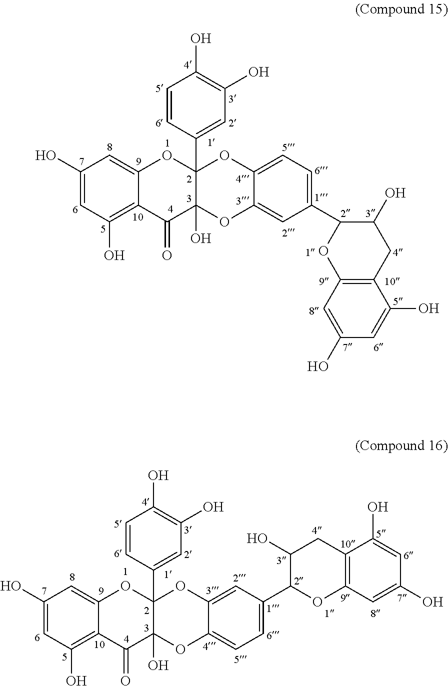

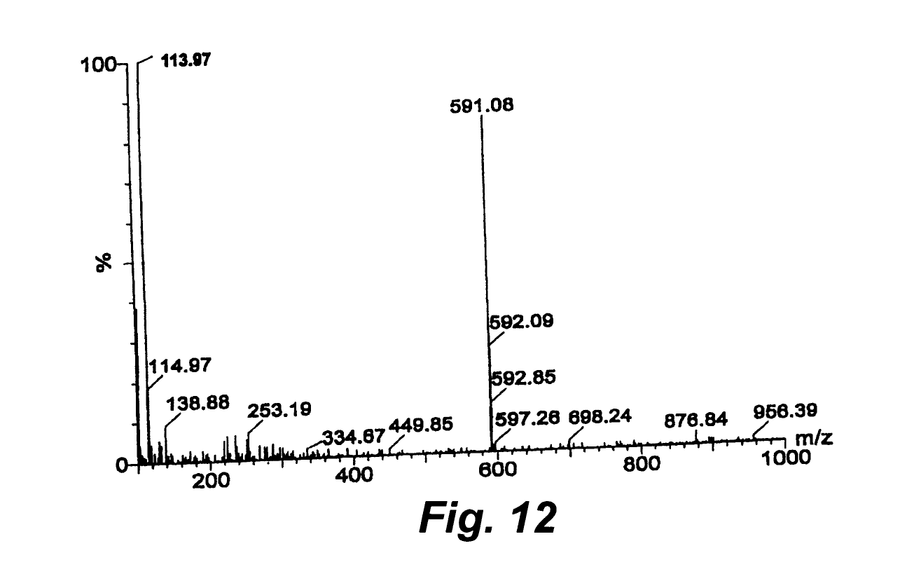

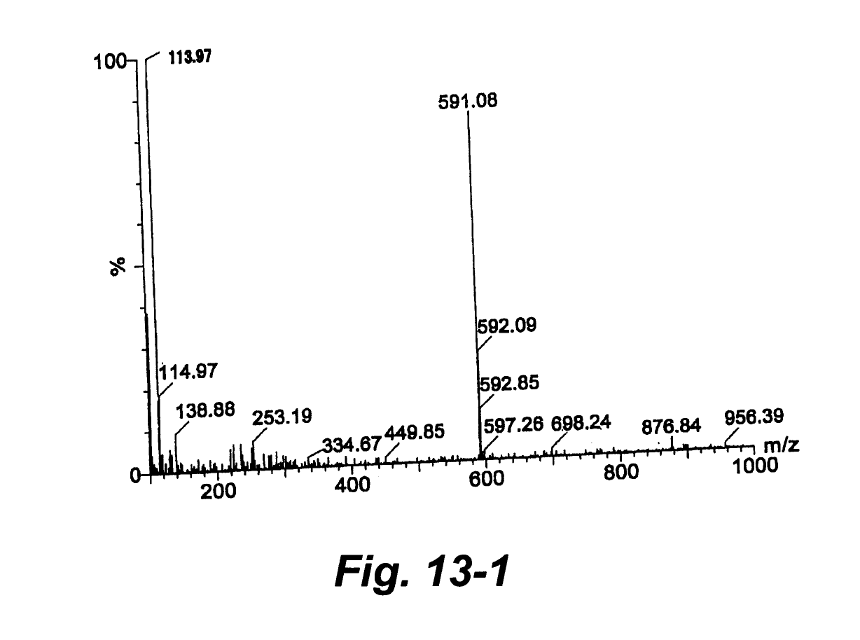

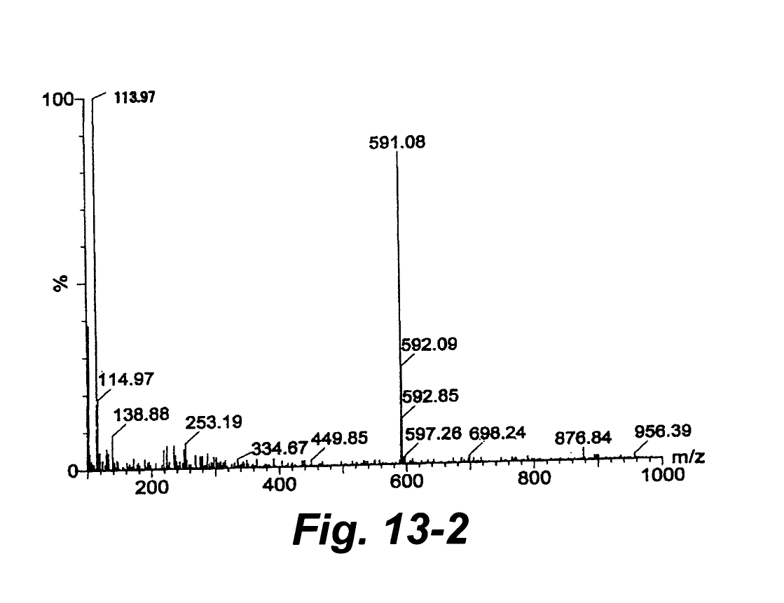

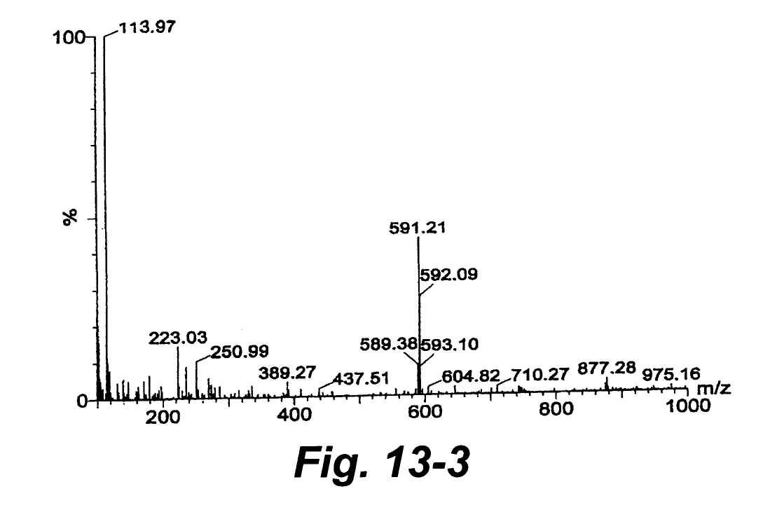

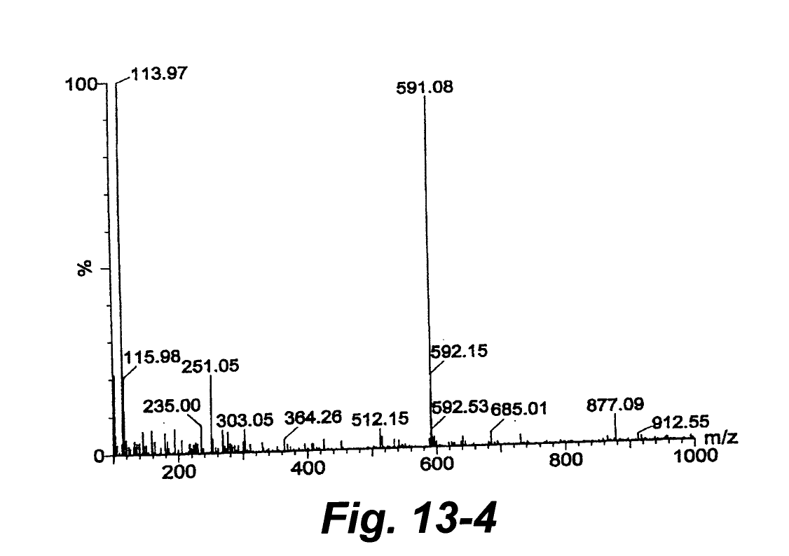

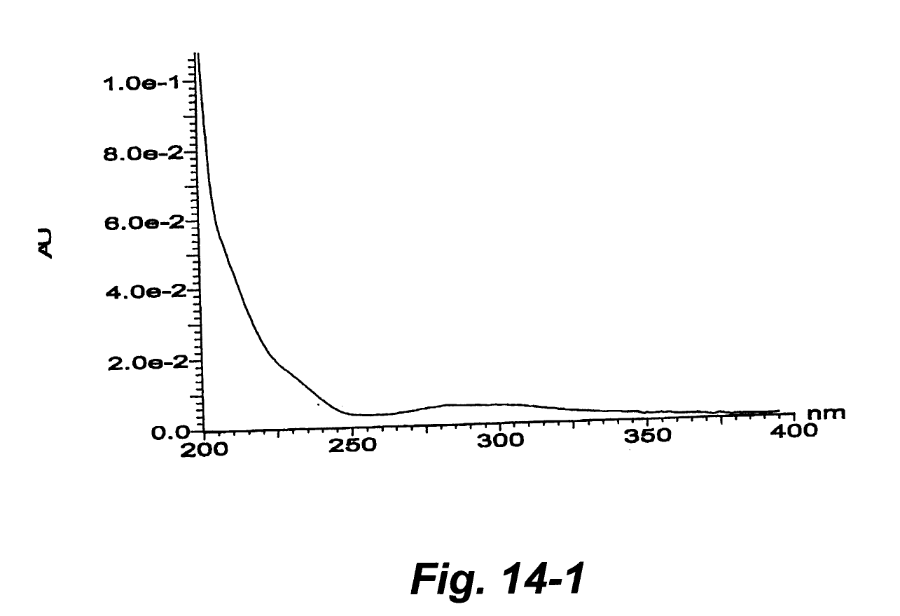

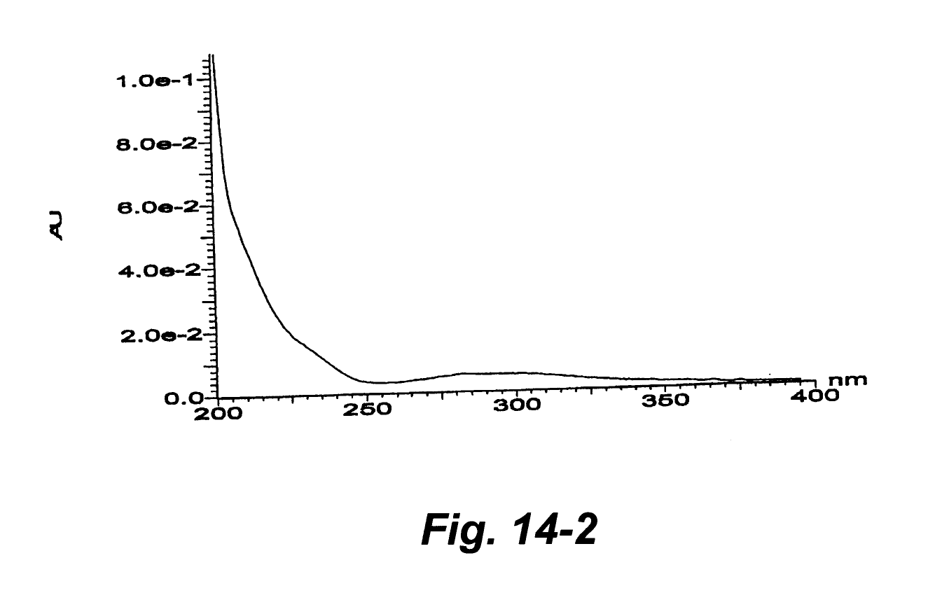

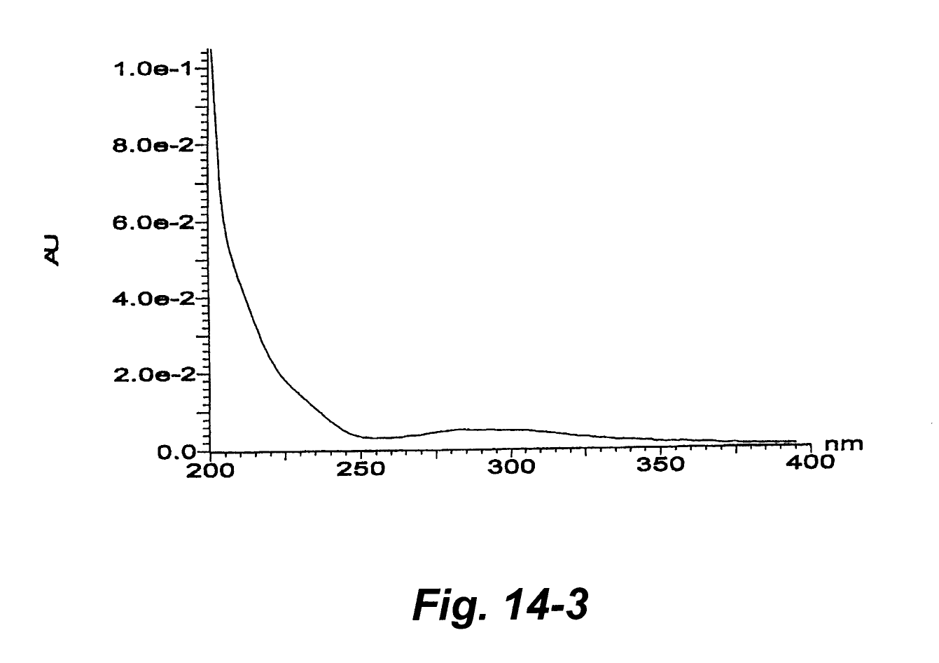

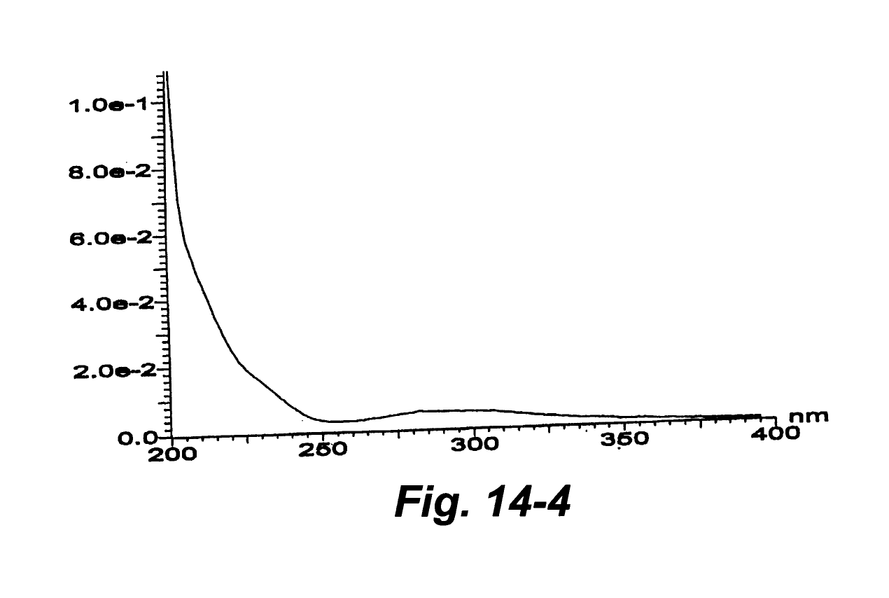

Quercetin 2 hydrate (720 mg) and catechin 1 hydrate (200 mg) were dissolved in purified water (1000 mL), and the mixture was warmed in a water bath at 50° C. for 30 minutes. Laccase DAIWA Y120 (100 mL, Amano Enzyme Inc.) prepared to 5 mg/mL was added and the mixture was stirred for 1 minute. Ethanol (1000 mL) was further added to quench the enzyme reaction. This reaction mixture was concentrated to dryness under reduced pressure, the sample was dissolved in purified water, and the solution was loaded on Sep-Pak C18 (Waters), washed with purified water, and eluted with 30% aqueous ethanol solution (100 mL). 30% Ethanol-eluted fraction was concentrated to dryness under reduced pressure, and purified by the following preparative HPLC. Four components having a molecular weight of 590 were obtained from the fractions with retention time 38.4 minutes, 39.2 minutes, 39.8 minutes, and 40.4 minutes. LC/MS was used for the detection of the component having a molecular weight of 590 by HPLC, and UV spectrum was simultaneously measured by a photodiode array detector (see FIGS. 12 to 14). The apparatus and measurement conditions of LC/MS and photodiode array detector are shown below.

HPLC apparatus:

pump: Hitachi Lachrom Elite L-2130 (manufactured by Hitachi, Ltd.)

detector: Hitachi Lachrom Elite L-2455 (manufactured by Hitachi, Ltd.)

autoinjector: Hitachi Lachrom Elite L-2200 (manufactured by Hitachi, Ltd.)

fraction collector: ADVANTEC SF-3120 (manufactured by ADVANTEC)

HPLC measurement condition:

column: CAPCELL PAK AQ S-5 μm, 20×250 mm (manufactured by Shiseido Co., Ltd.)

solvent: solution of 0.05% trifluoroacetic acid and 25% acetonitrile in water

flow rate: 6 mL/minute

detection wavelength: 280 nm

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid and 10% acetonitrile in water, SOLUTION B solution of 0.05% trifluoroacetic acid and 80% acetonitrile in water

¹H-NMR (400 MHz, MeOH-d4): shown in Table 1.

¹³C-NMR (100 MHz, MeOH-d4): shown in Table 2.

As the NMR measurement device, Bruker Avance 400 (400 MHz, Bruker BioSpin KK) was used. The attribution of which carbon to which each proton is bonded was determined on the basis of the measurement by two-dimensional NMR (HSQC), and the assumed whole structure was attributed by two-dimensional NMR (HMBC). From the results of NMR analysis of each component, the difference was assumed to be any of (compound 15) or (compound 16) in a flat plane structure; however, (compound 15) and (compound 16) cannot be distinguished by NMR analysis.

### Synthesis Example 6

### Synthesis Example of Compounds with the Structures Shown Below

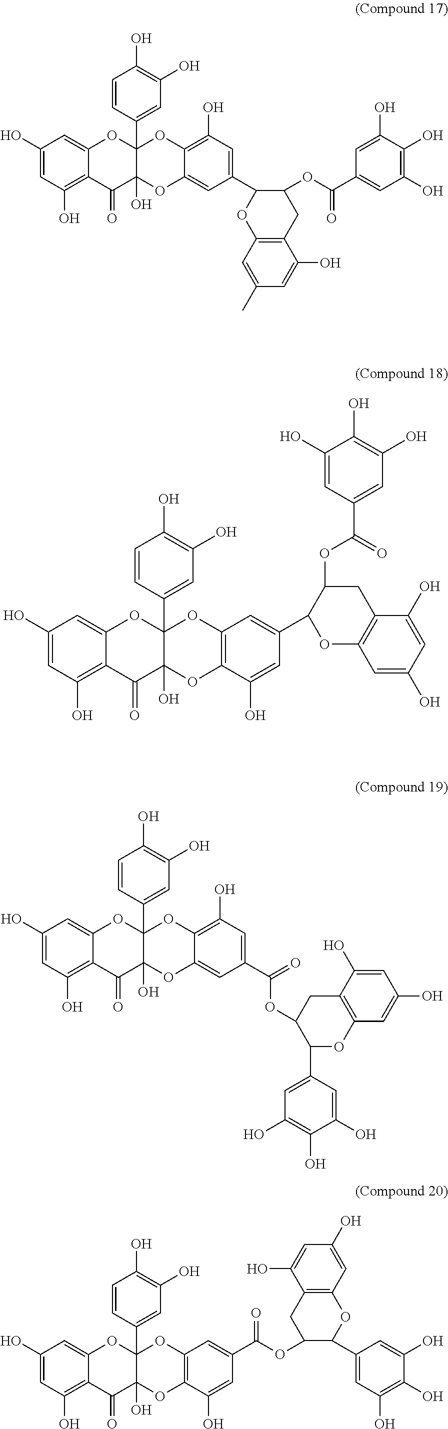

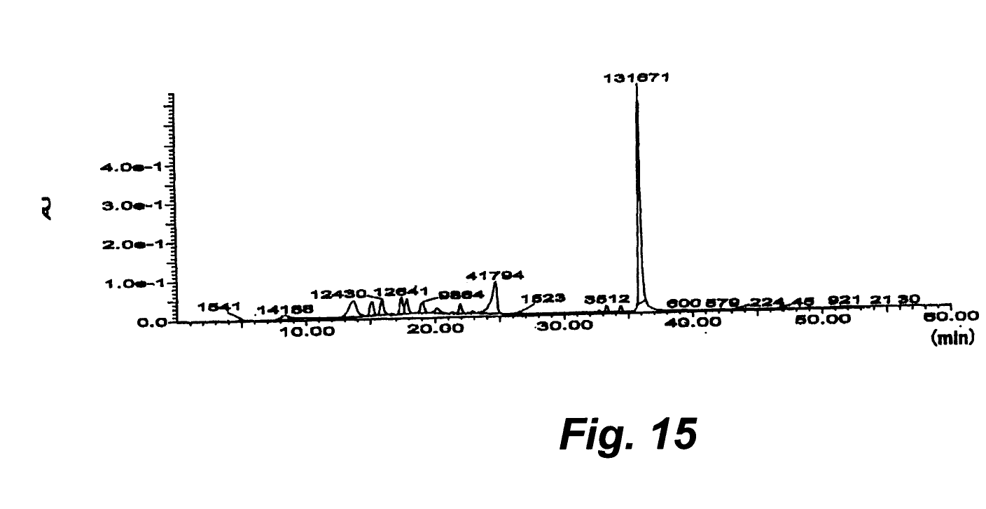

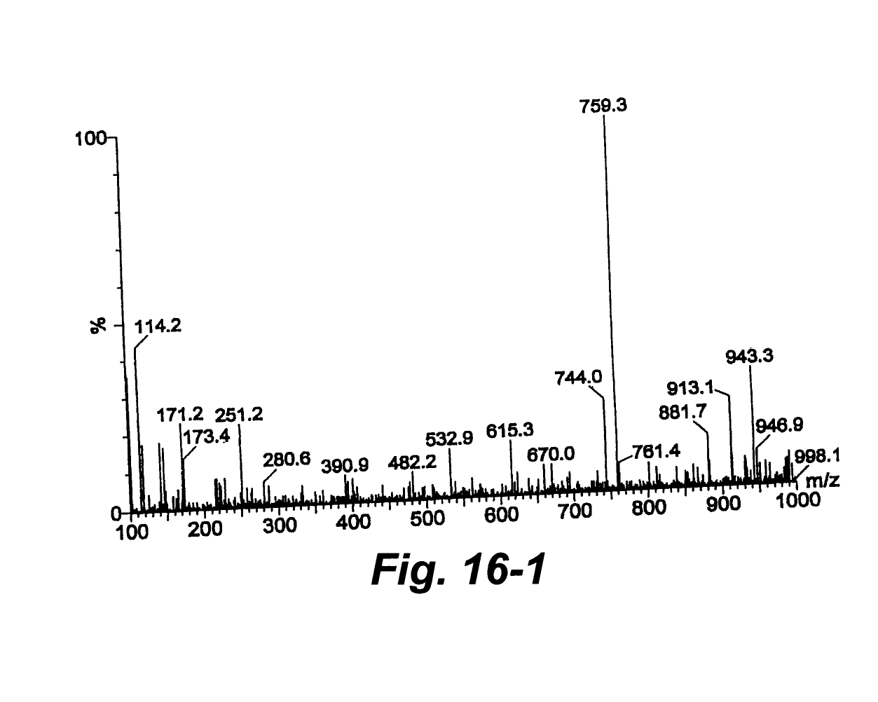

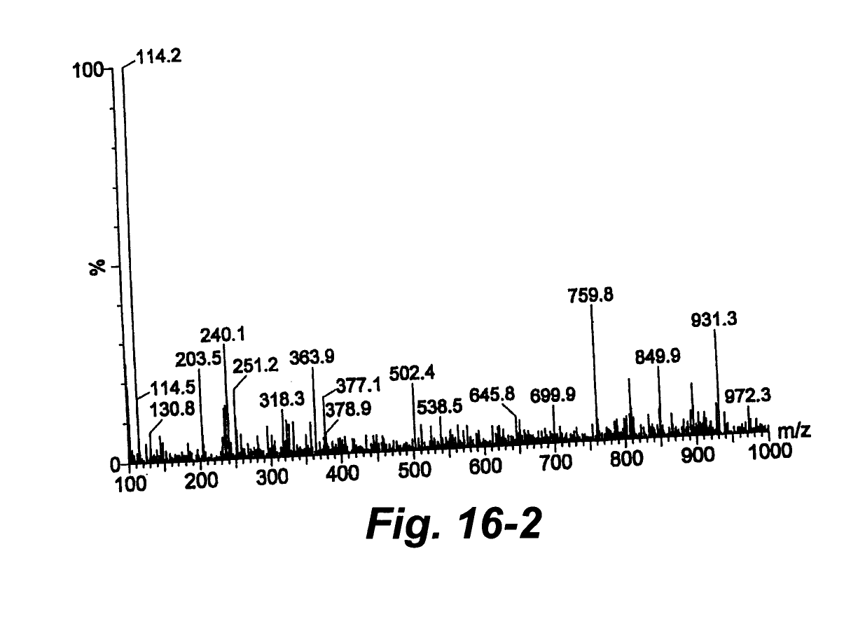

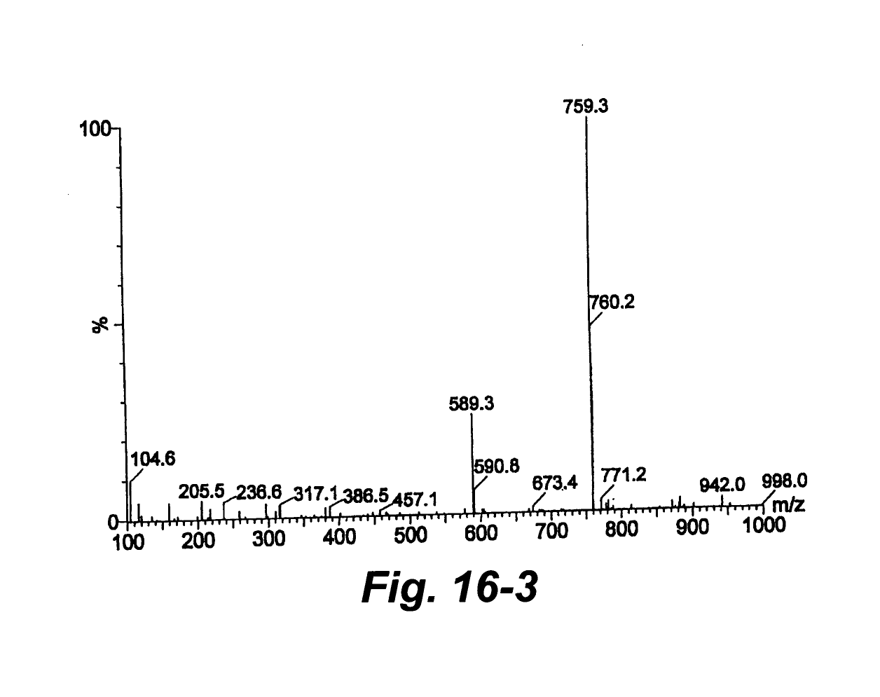

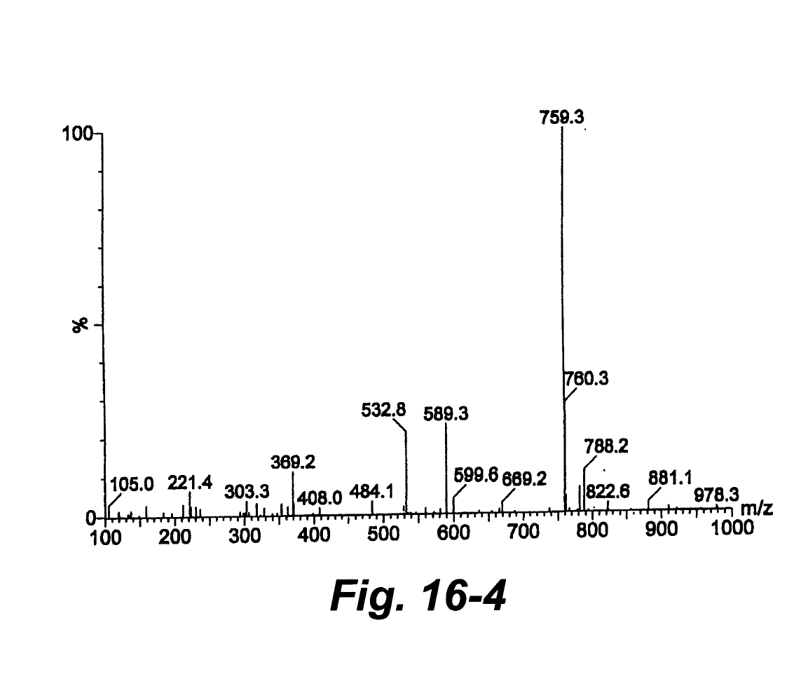

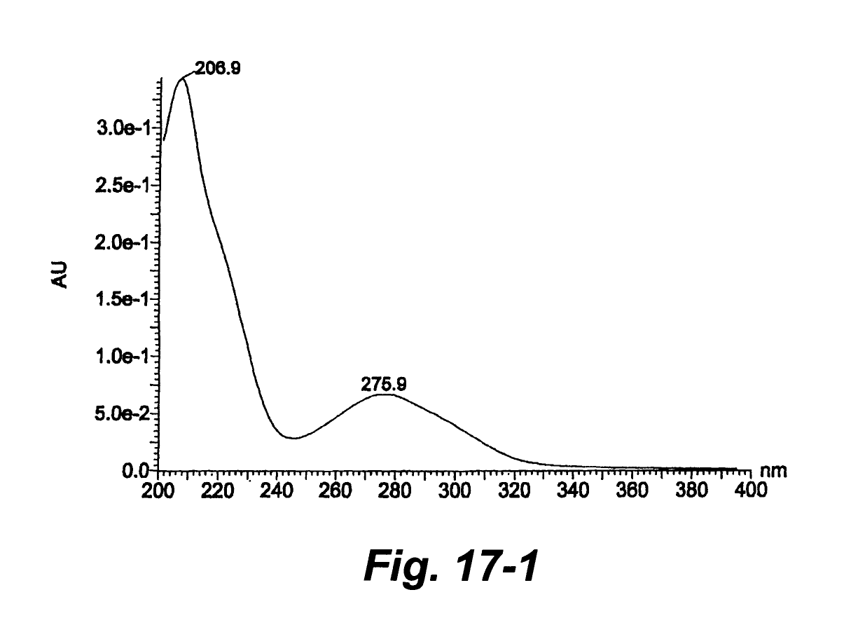

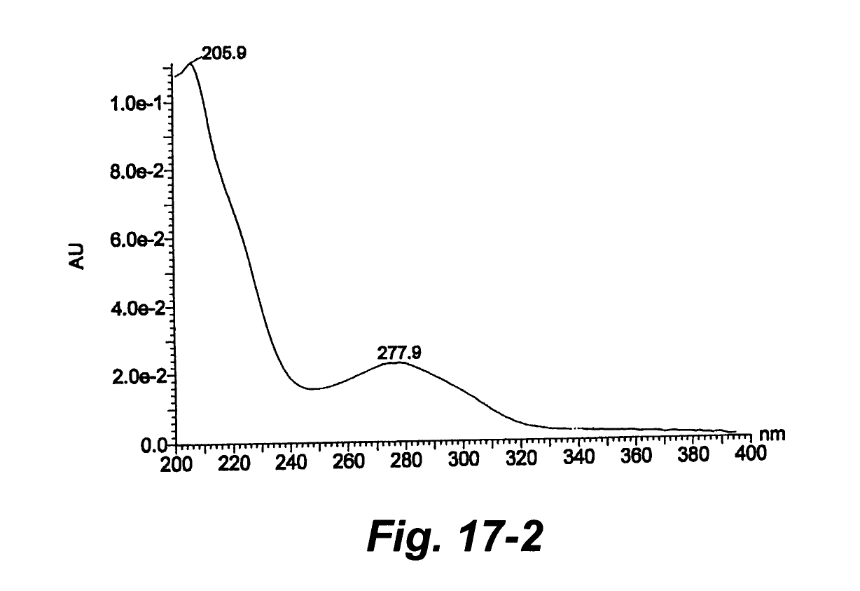

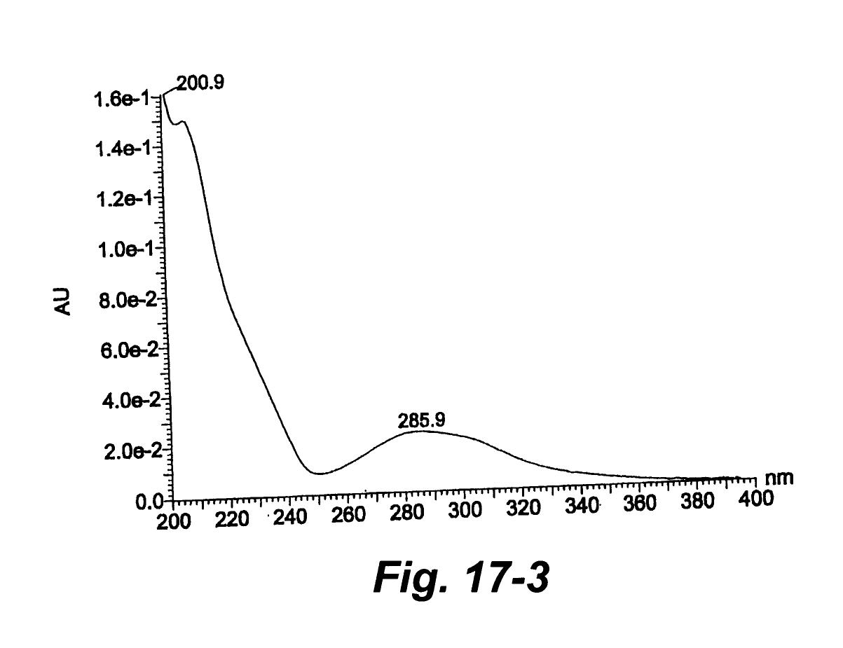

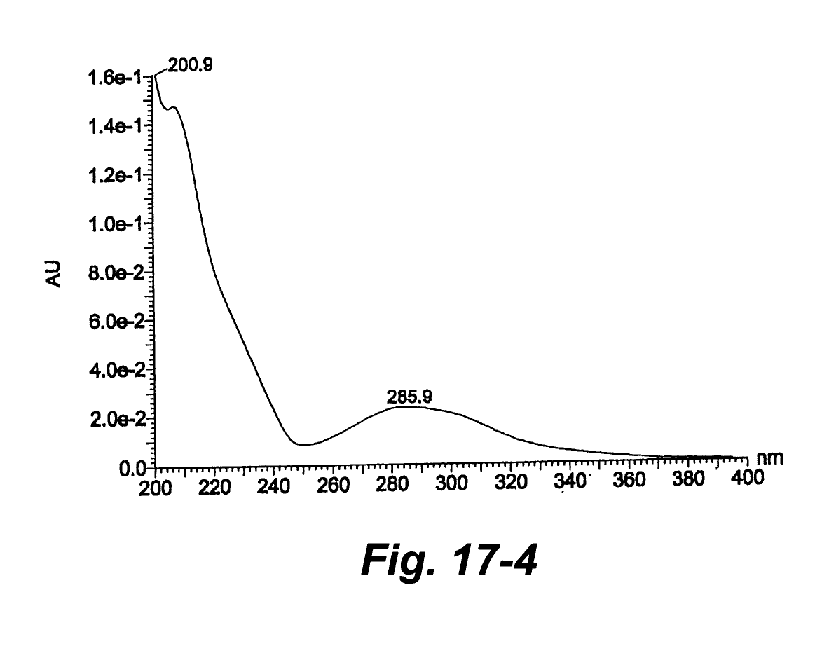

Quercetin 2 hydrate (100 μg) and (−)-epigallocatechin gallate (100 μg) were dissolved in purified water (200 μL), and the mixture was warmed in an incubator at 50° C. for 1 minute. Laccase DAIWA Y120 (10 μL, Amano Enzyme Inc.) prepared to 5 mg/mL was added and the mixture was stirred for 1 minute. Ethanol (200 μL) was further added to quench the enzyme reaction. This reaction mixture was analyzed by LC/MS, and 4 components having a molecular weight of 758 could be confirmed in the fractions with retention time 19.0 minutes, 20.0 minutes, 33.3 minutes, and 34.4 minutes. UV spectrum was simultaneously measured by a photodiode array detector (see FIGS. 15 to 17). As a result of the analysis of UV spectrum of each component, the absorption around 370 nm derived from ring C of quercetin disappeared, and only the absorption around 280 nm derived from ring A, ring B and a galloyl group was strongly detected, which confirms that the structures from (compound 17) to (compound 20) were produced. As for the peaks with retention time 33.3 minutes and 34.4 minutes, since a fragment ion m/z 589.3 due to the dissociation of the galloyl group was detected by mass spectrum, the peaks of retention times 33.3 minutes and 34.4 minutes were assumed to be (compound 17) and (compound 18), respectively. The apparatus and measurement conditions of LC/MS and photodiode array detector are shown below.

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid and 10% acetonitrile in water, SOLUTION B solution of 0.05% trifluoroacetic acid and 80% acetonitrile in water

### Synthesis Example 7

### Synthesis Example of Compounds with the Structures Shown Below

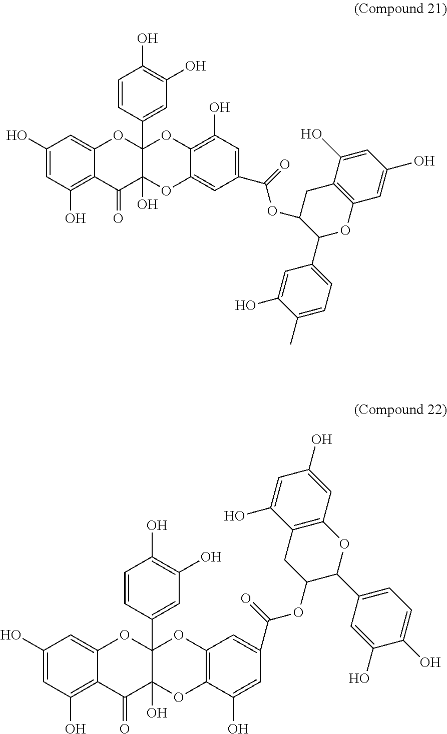

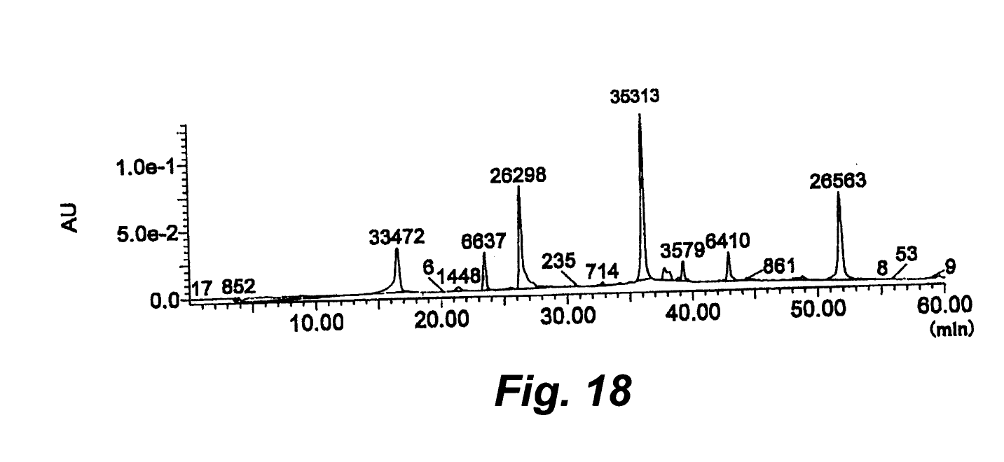

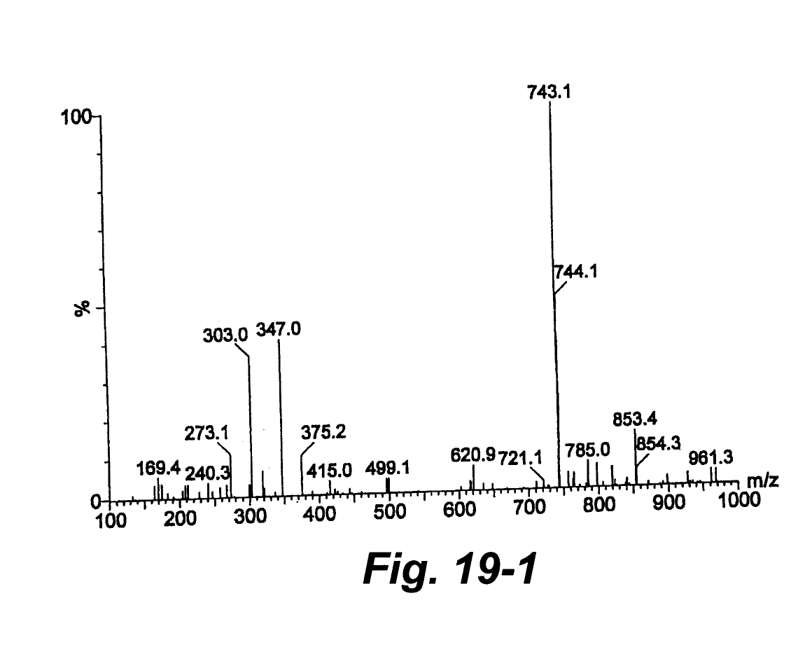

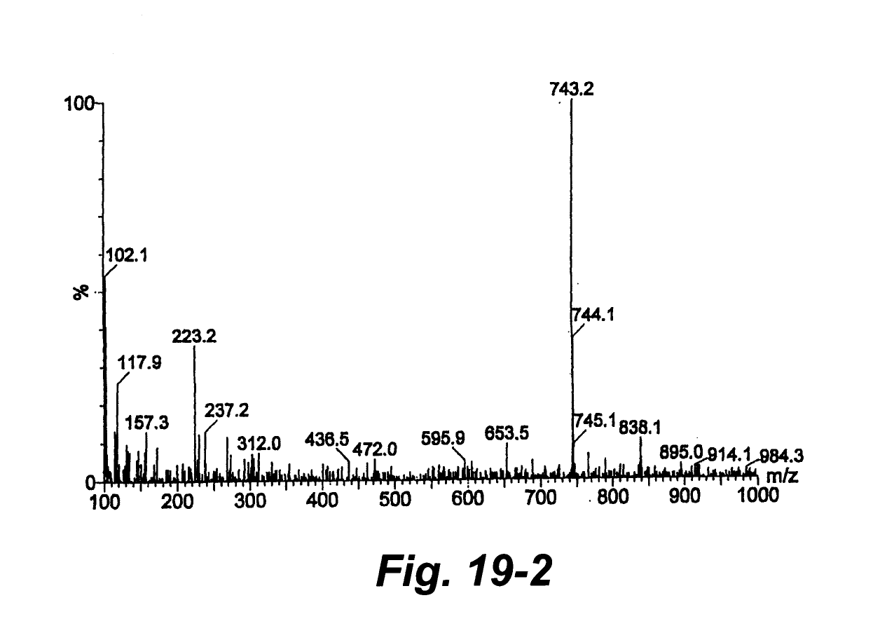

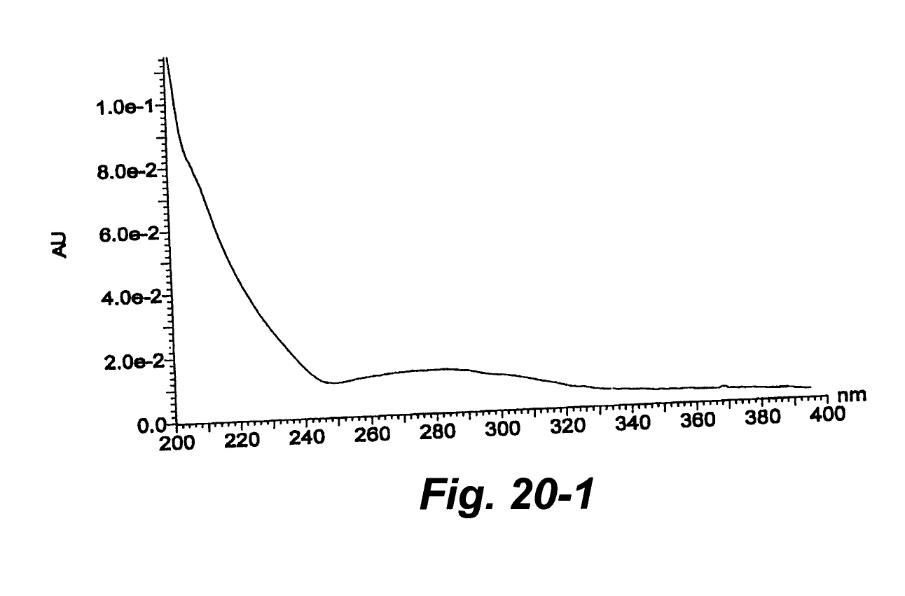

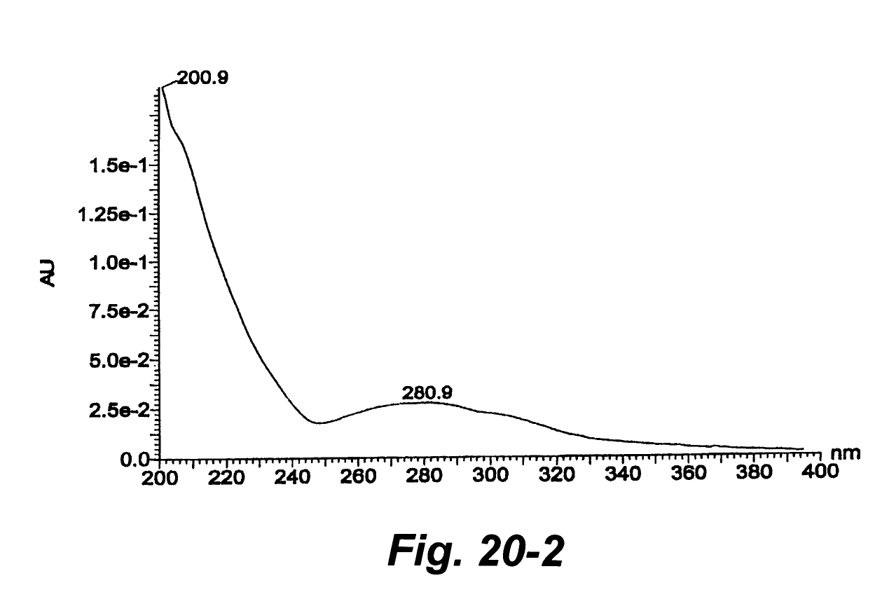

Quercetin 2 hydrate (100 μg) and catechin gallate 1 hydrate (100 μg) were dissolved in purified water (200 μL), and the mixture was warmed in an incubator at 50° C. for 1 minute. Laccase DAIWA Y120 (10 μL, Amano Enzyme Inc.) prepared to 5 mg/mL was added and the mixture was stirred for 1 minute. Ethanol (200 μL) was further added to quench the enzyme reaction. This reaction mixture (1 μL) was analyzed by LC/MS, and two components having a molecular weight of 742 could be confirmed in the fractions with retention time 38.2 minutes and 39.1 minutes. UV spectrum was simultaneously measured by a photodiode array detector (see FIGS. 18 to 20). The apparatus and measurement conditions of LC/MS and photodiode array detector are shown below. As a result of the analysis of UV spectrum of each component, the absorption around 370 nm derived from ring C of quercetin disappeared, only the absorption around 280 nm derived from ring A, ring B and a galloyl group was strongly detected, and a fragment showing dissociation of the galloyl group was not observed, which confirms that the structures of (compound 21) and (compound 22), which were bonded to a galloyl group of catechin gallate, were produced.

### Synthesis Example 8

### Synthesis Example of Compounds with the Structures Shown Below

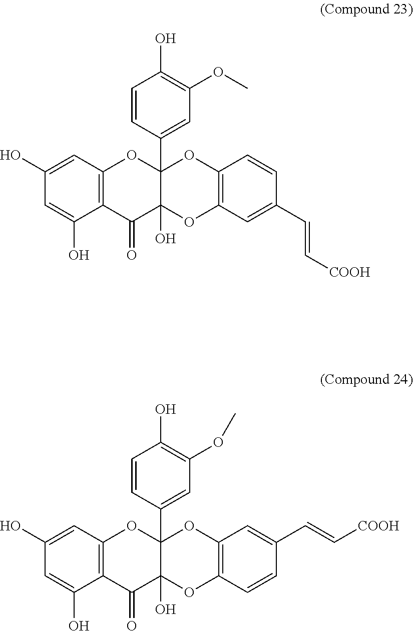

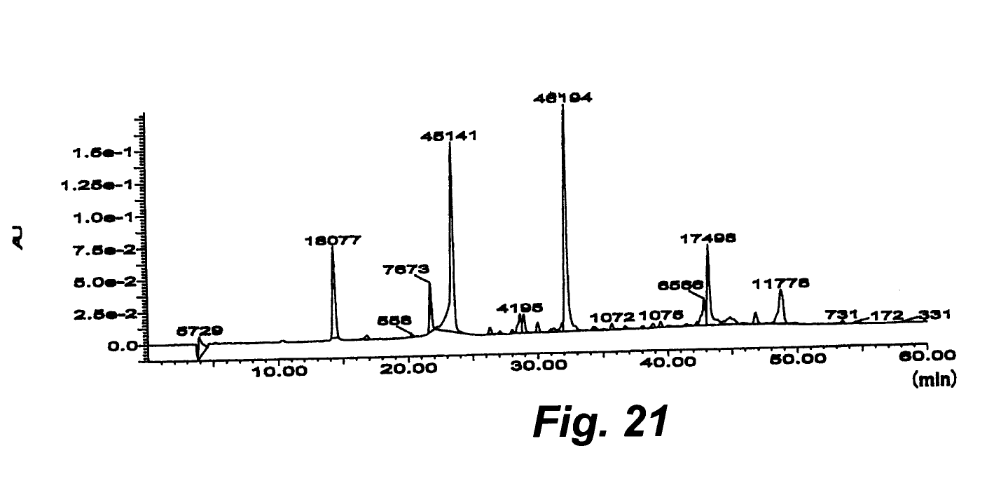

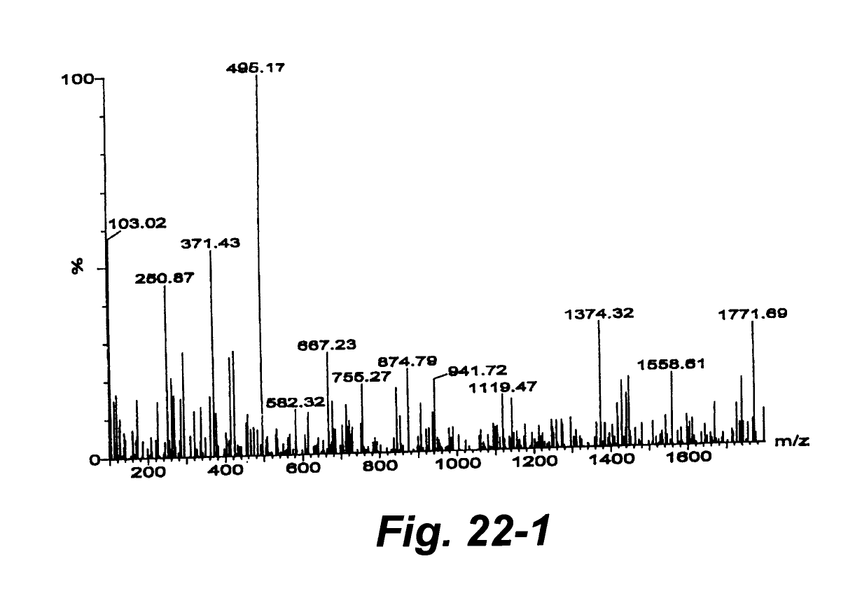

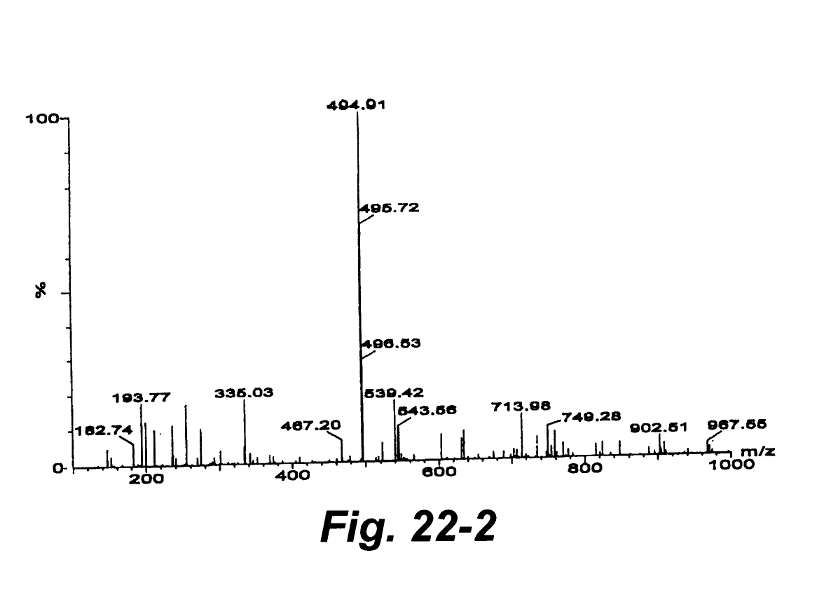

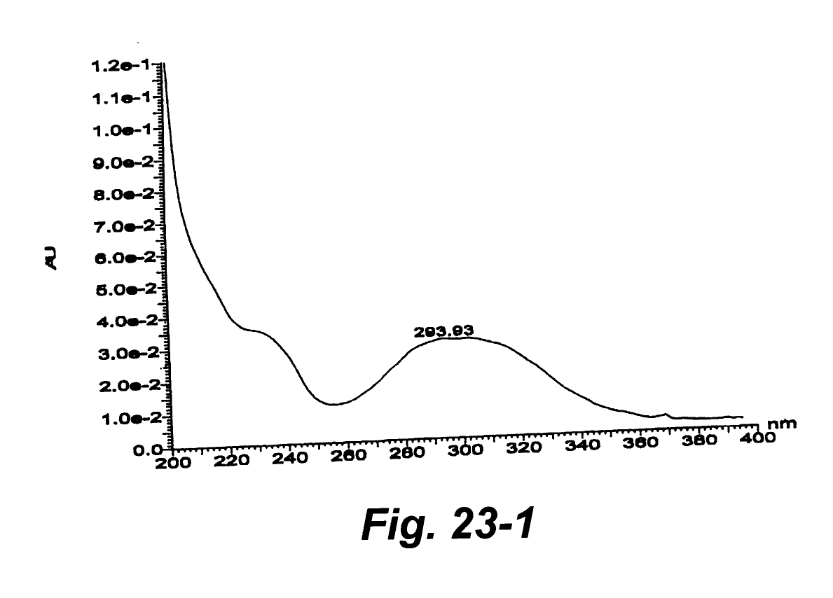

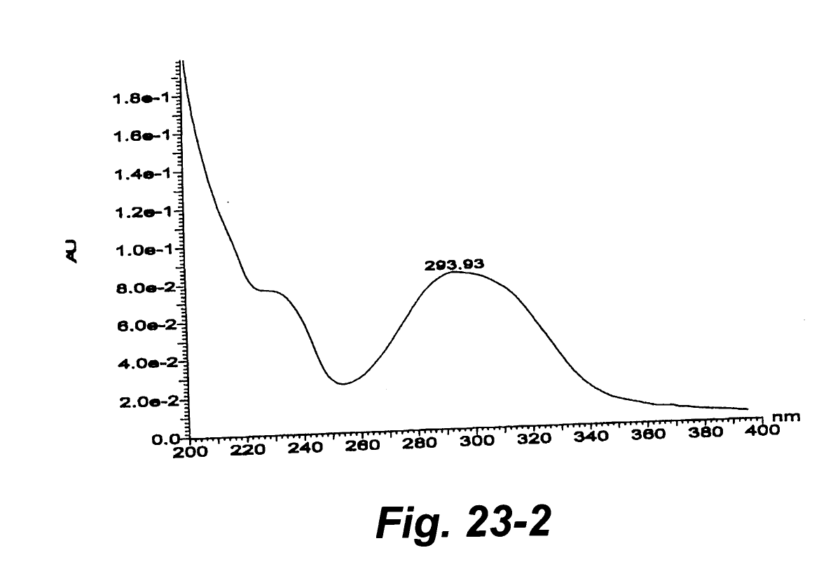

Isorhamnetin (100 μg) and caffeic acid (100 μg) were dissolved in purified water (200 μL), and the mixture was warmed in an incubator at 50° C. for 1 minute. Laccase DAIWA Y120 (10 μL, Amano Enzyme Inc.) prepared to 5 mg/mL was added and the mixture was stirred for 1 minute. Ethanol (200 μL) was further added to quench the enzyme reaction. This reaction mixture (1 μL) was analyzed by LC/MS, and two components having a molecular weight of 494 could be confirmed in the fractions with retention time 43.0 minutes and 43.4 minutes. UV spectrum was simultaneously measured by a photodiode array detector (see FIGS. 21 to 23). From the results of UV spectrum analysis of each component, the absorption around 370 nm derived from ring C of isorhamnetin disappeared, and only the absorption around 280 nm derived from ring A and ring B was strongly detected, which confirms that the structure of (compound 23) or (compound 24) was produced. The apparatus and measurement conditions of LC/MS and photodiode array detector are shown below.

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid and 10% acetonitrile in water, SOLUTION B solution of 0.05% trifluoroacetic acid and 80% acetonitrile in water

### Synthesis Example 9

### Synthesis Example of Compounds with the Structures Shown Below

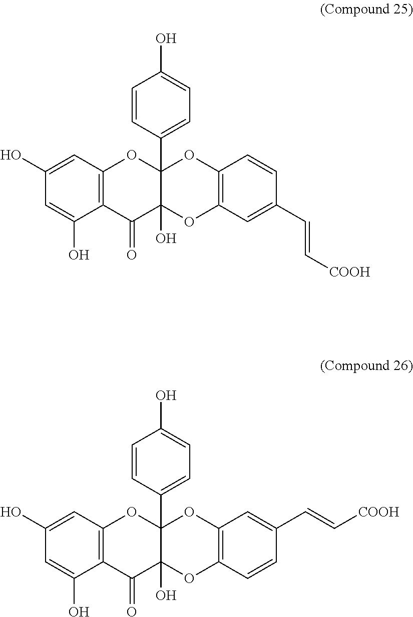

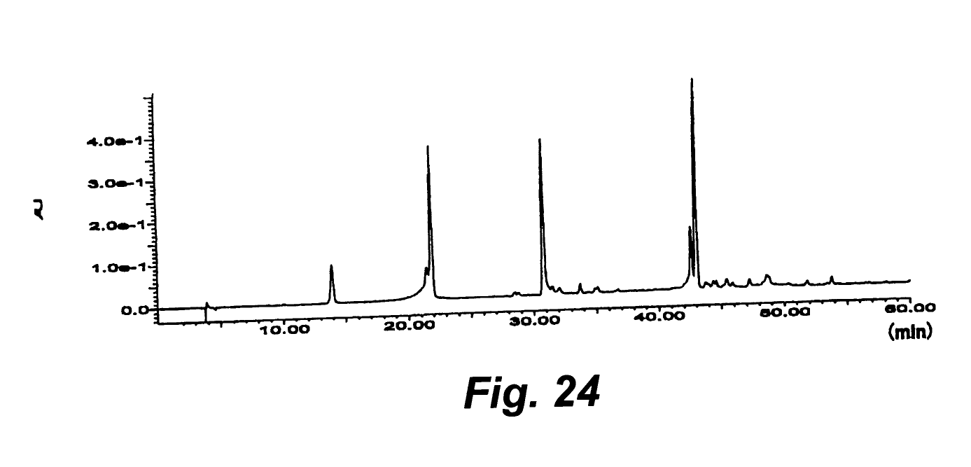

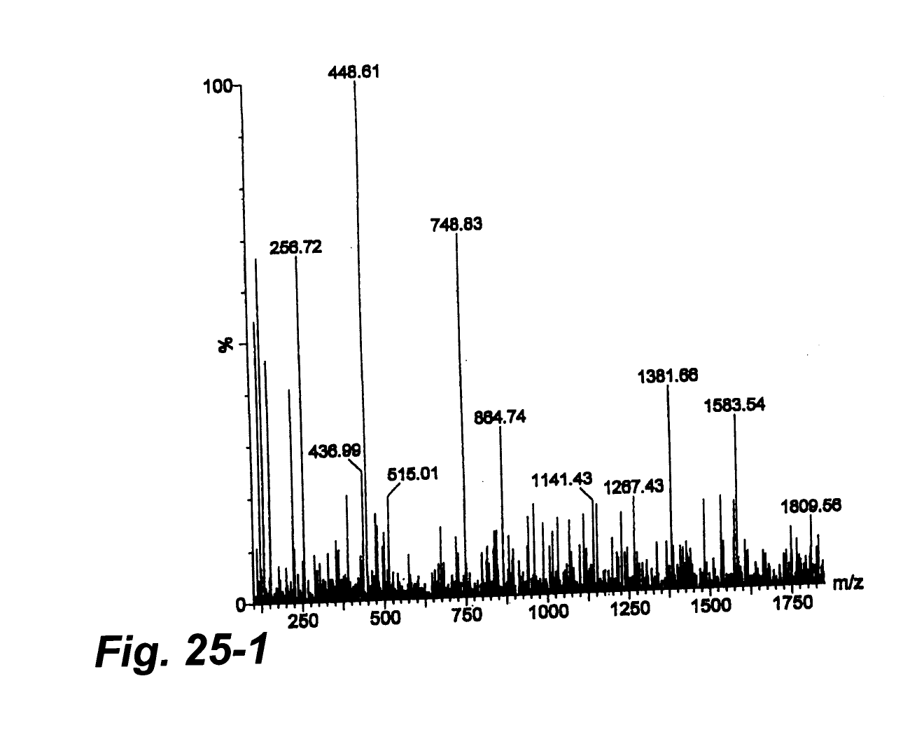

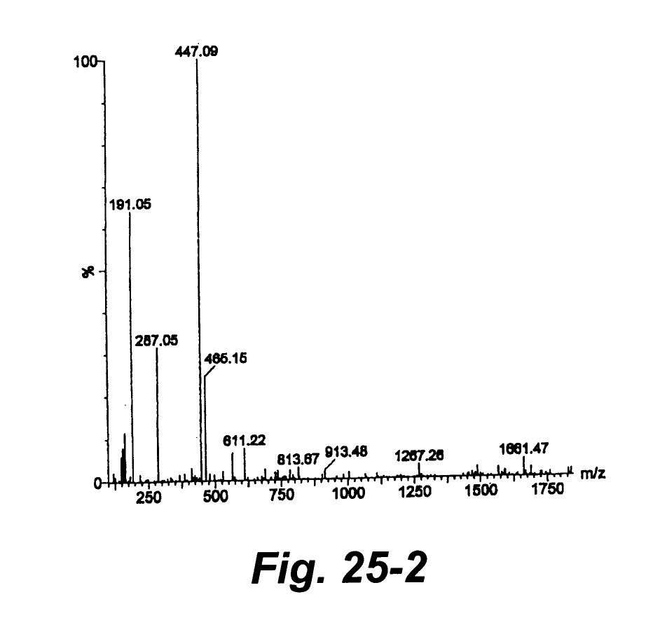

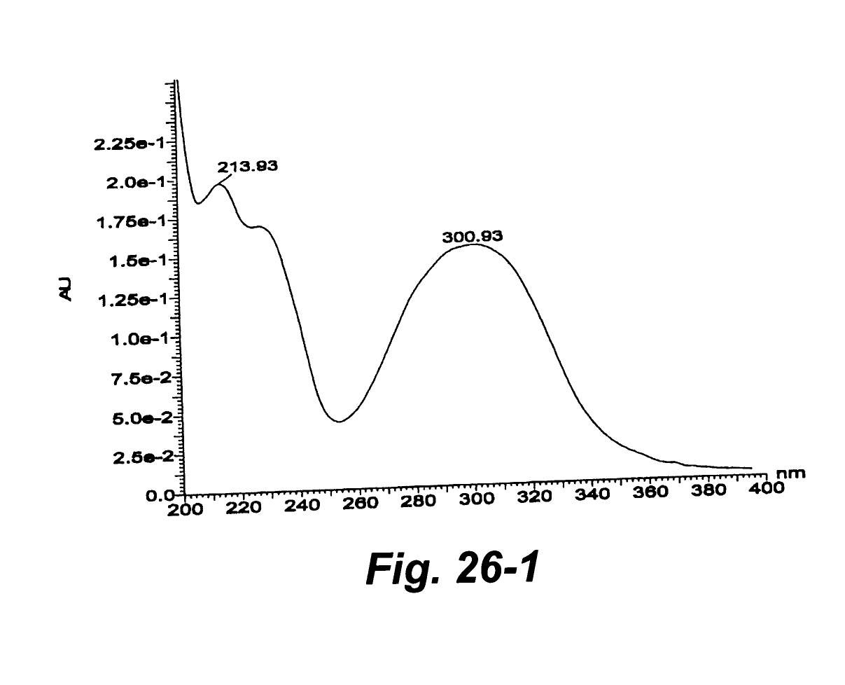

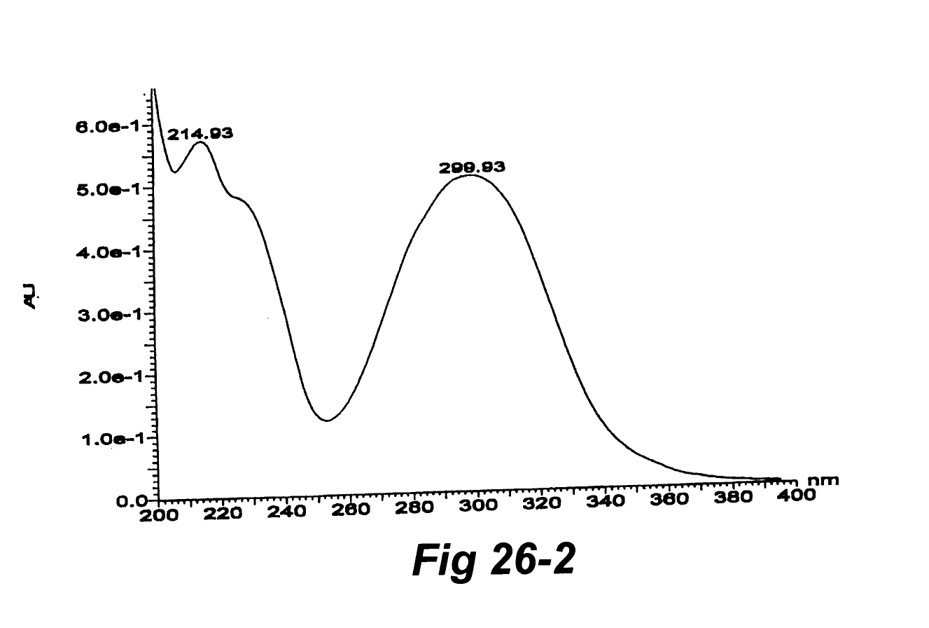

Kaempferol (100 μg) and caffeic acid (100 μg) were dissolved in purified water (200 μL), and the mixture was warmed in an incubator at 50° C. for 1 minute. Laccase DAIWA Y120 (10 μL, Amano Enzyme Inc.) prepared to 5 mg/mL was added and the mixture was stirred for 1 minute. Ethanol (200 μL) was further added to quench the enzyme reaction. This reaction mixture (1 μL) was analyzed by LC/MS, and two components having a molecular weight of 464 could be confirmed in the fractions with retention time 42.7 minutes and 43.2 minutes. UV spectrum was simultaneously measured by a photodiode array detector (see FIGS. 24 to 26). As a result of the UV spectrum analysis of each component, the absorption around 370 nm derived from ring C of kaempferol disappeared, and only the absorption around 280 nm derived from ring A and ring B was strongly detected, which confirms that the structure of (compound 25) or (compound 26) was produced. The apparatus and measurement conditions of LC/MS and photodiode array detector are shown below.

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid and 10% acetonitrile in water, SOLUTION B solution of 0.05% trifluoroacetic acid and 80% acetonitrile in water

### Synthesis Example 10

### Synthesis Example of Compounds with the Structures Shown Below

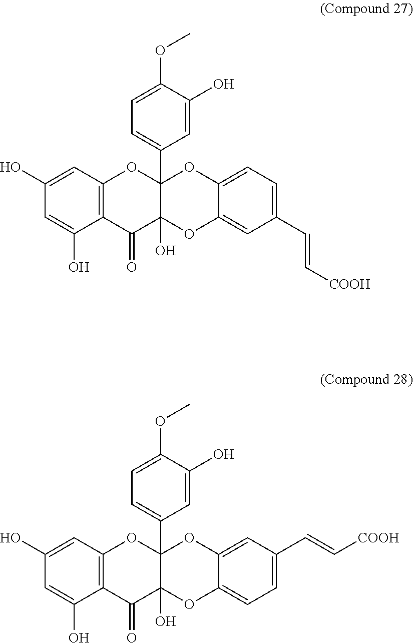

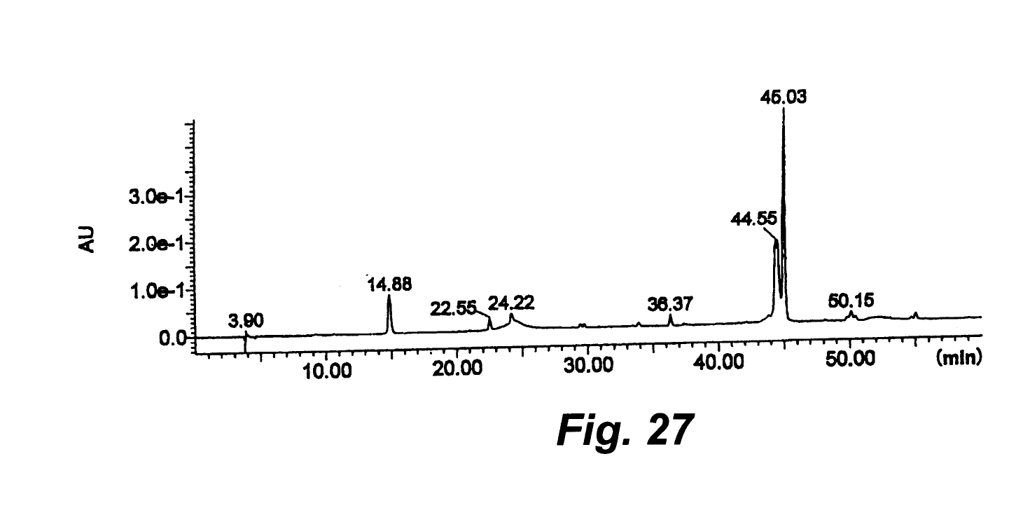

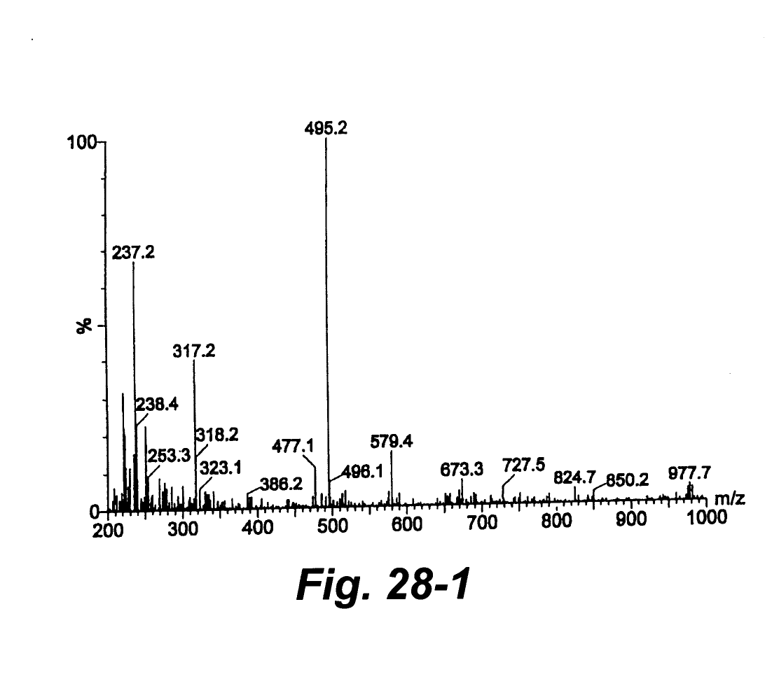

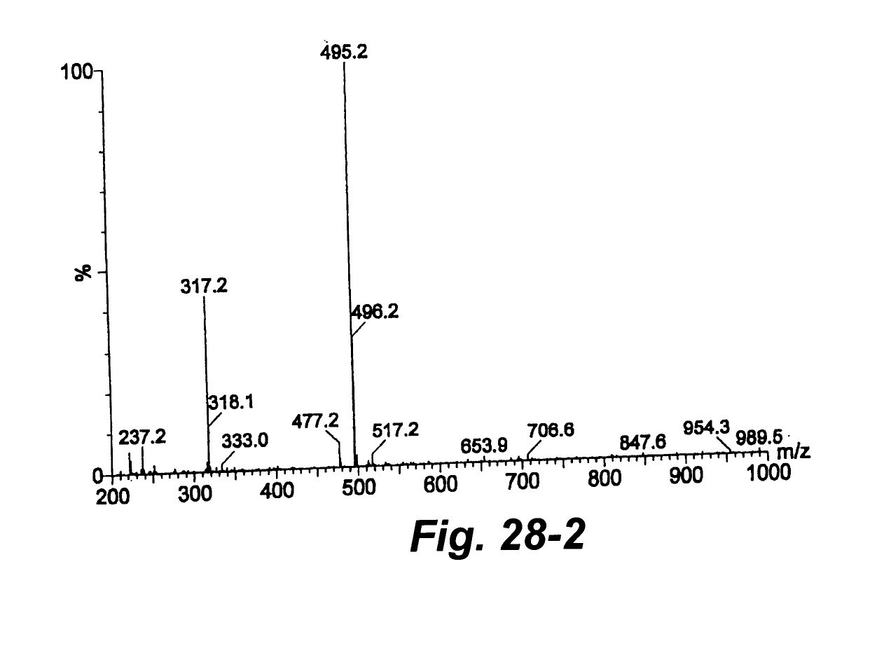

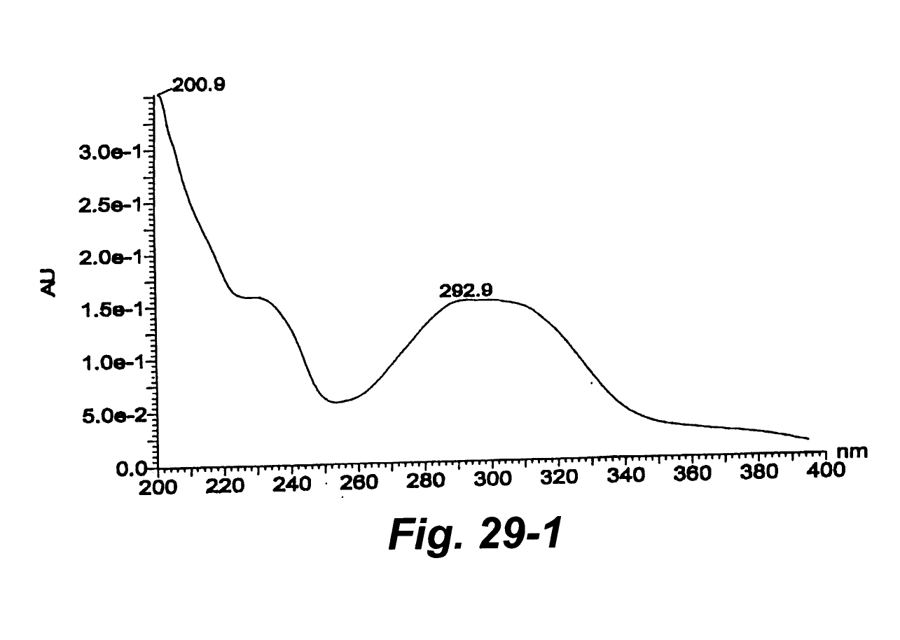

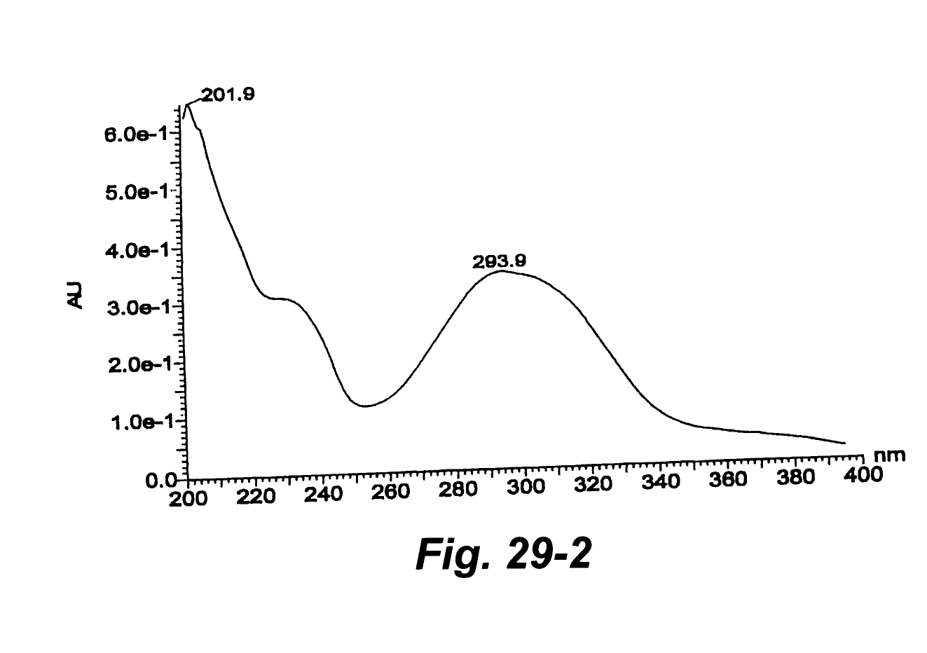

Tamarixetin (100 μg) and caffeic acid (100 μg) were dissolved in purified water (200 μL), and the mixture was warmed in an incubator at 50° C. for 1 minute. Laccase DAIWA Y120 (10 μL, Amano Enzyme Inc.) prepared to 5 mg/mL was added and the mixture was stirred for 1 minute. Ethanol (200 μL) was further added to quench the enzyme reaction. This reaction mixture (1 μl) was analyzed by LC/MS, and two components having a molecular weight of 494 could be confirmed in the fractions with retention time 44.5 minutes and 45.0 minutes. UV spectrum was simultaneously measured by a photodiode array detector (see FIGS. 27 to 29). The apparatus and measurement conditions of LC/MS and photodiode array detector are shown below.

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid and 10% acetonitrile in water, SOLUTION B solution of 0.05% trifluoroacetic acid and 80% acetonitrile in water

As a result of the UV spectrum analysis of each component, the absorption around 370 nm derived from ring C of tamarixetin disappeared, and only the absorption around 280 nm derived from ring A and ring B was strongly detected, which confirms that the structure of (compound 27) or (compound 28) was produced.

### Synthesis Example 11

### Synthesis Example of Compounds with the Structures Shown Below

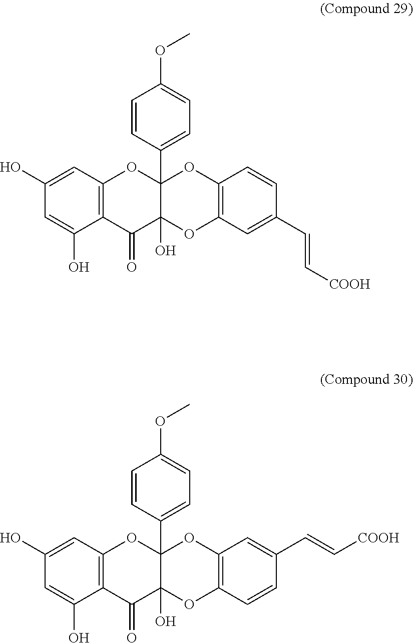

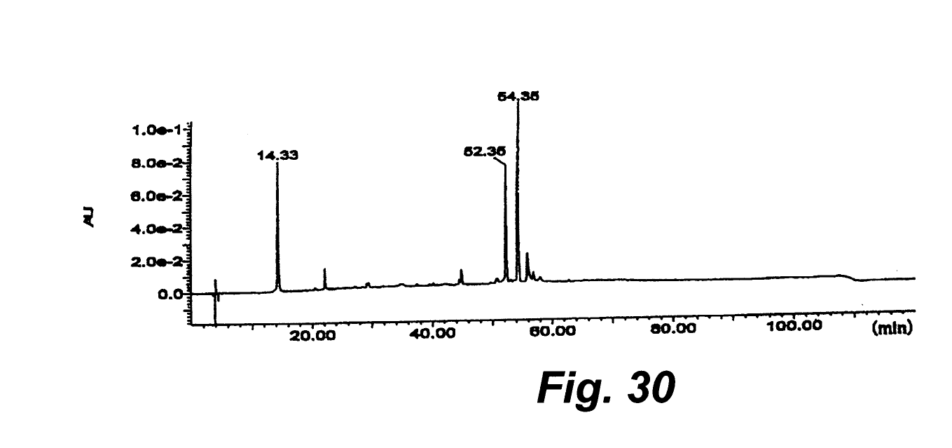

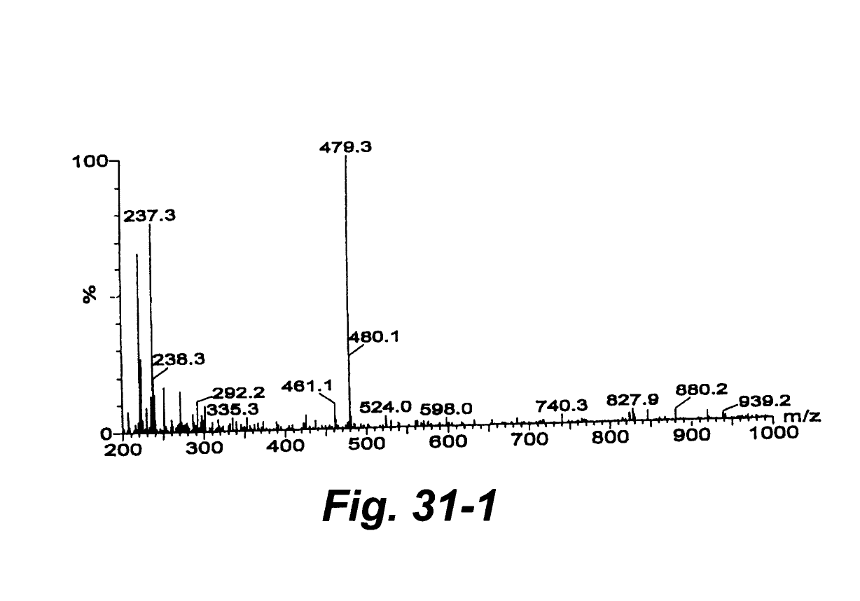

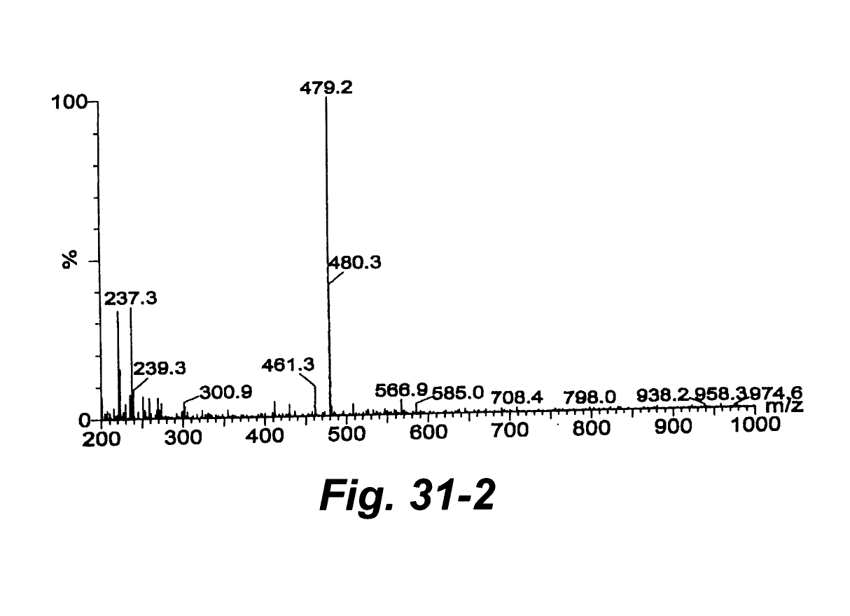

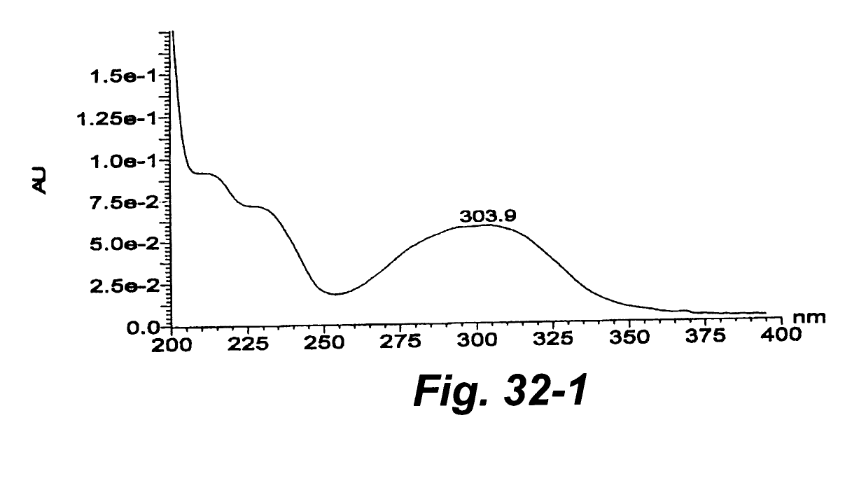

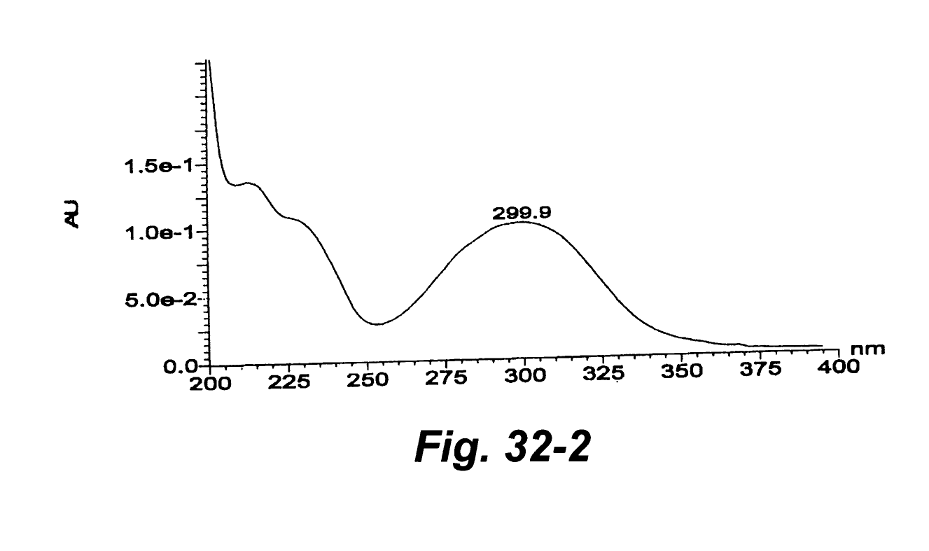

Kaempferide (100 μg) and caffeic acid (100 μg) were dissolved in purified water (200 μL), and the mixture was warmed in an incubator at 50° C. for 1 minute. Laccase DAIWA Y120 (10 μL, Amano Enzyme Inc.) prepared to 5 mg/mL was added and the mixture was stirred for 1 minute. Ethanol (200 μL) was further added to quench the enzyme reaction. This reaction mixture (1 μL) was analyzed by LC/MS, and two components having a molecular weight of 478 could be confirmed in the fractions is with retention time 52.4 minutes and 54.4 minutes. UV spectrum was simultaneously measured by a photodiode array detector (see FIGS. 30 to 32). As a result of the UV spectrum analysis of each component, the absorption around 370 nm derived from ring C of kaempferide disappeared, and only the absorption around 280 nm derived from ring A and ring B was strongly detected, which confirms that the structure of (compound 29) or (compound 30) was produced. The apparatus and measurement conditions of LC/MS and photodiode array detector are shown below.

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid and 10% acetonitrile in water, SOLUTION B solution of 0.05% trifluoroacetic acid and 80% acetonitrile in water

### Synthesis Example 12

### Synthesis Example of Compounds with the Structures Shown Below

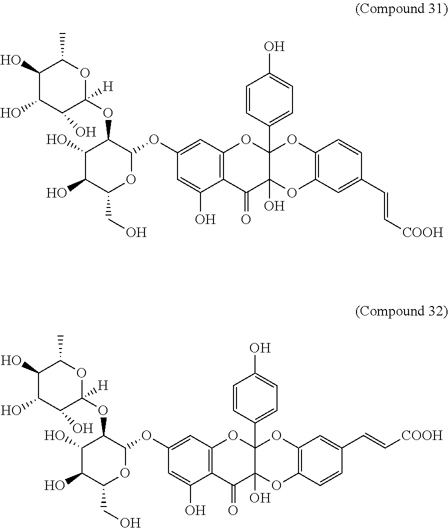

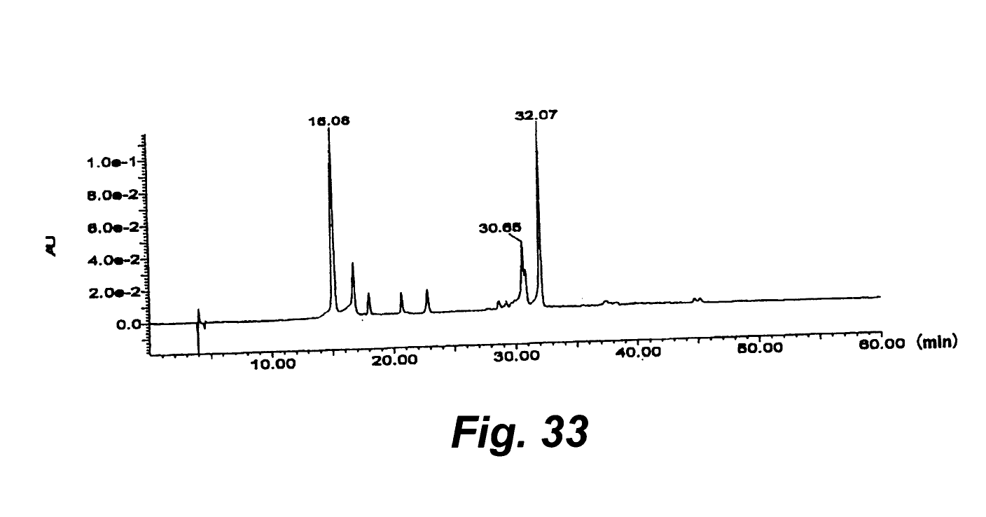

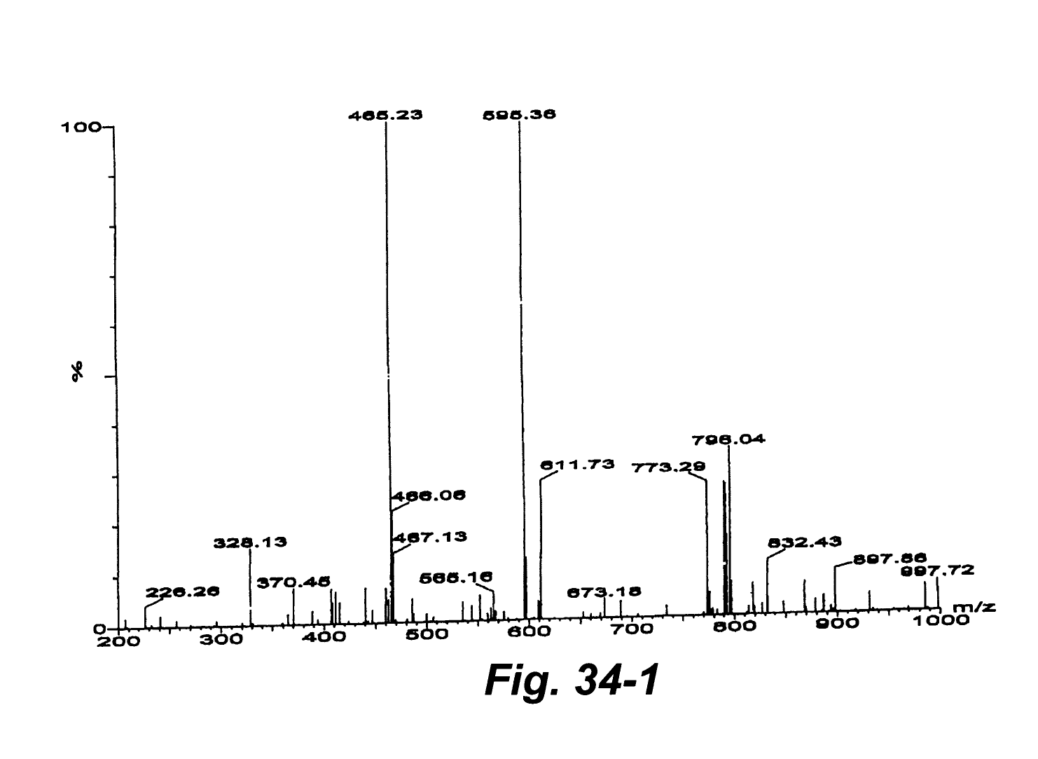

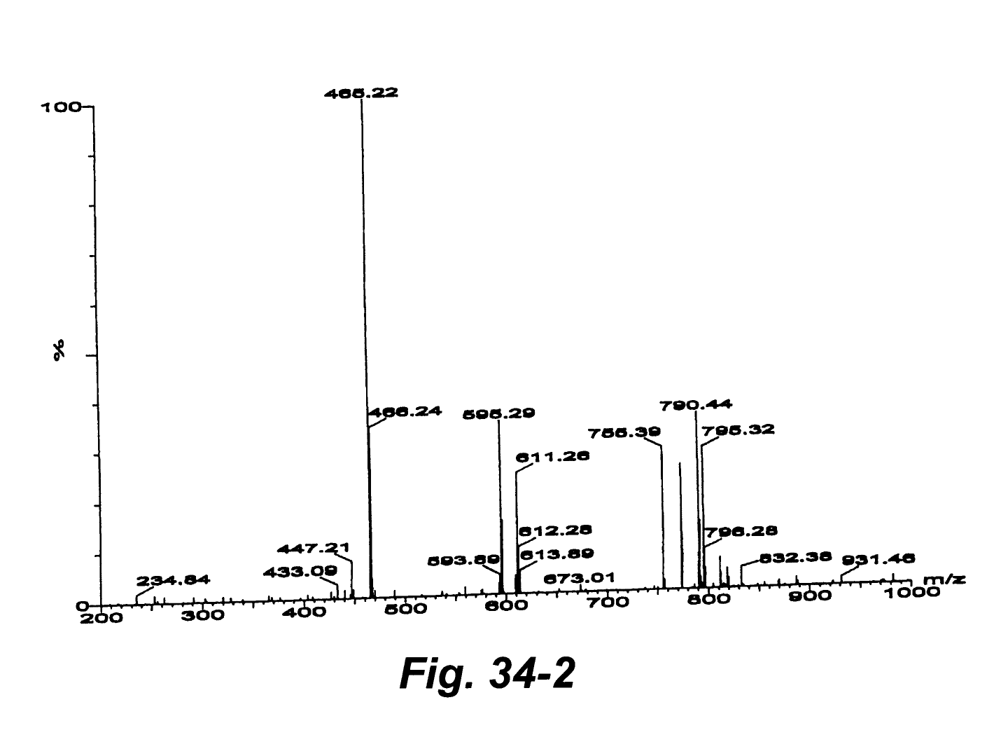

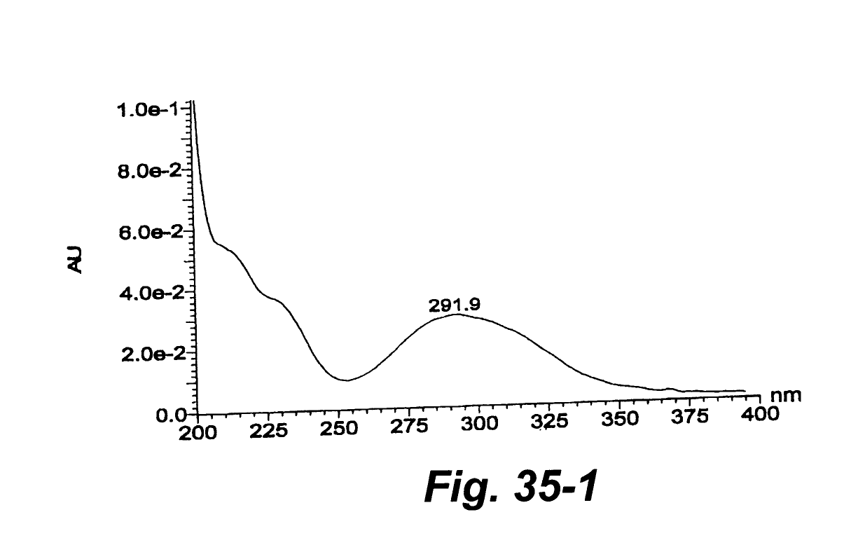

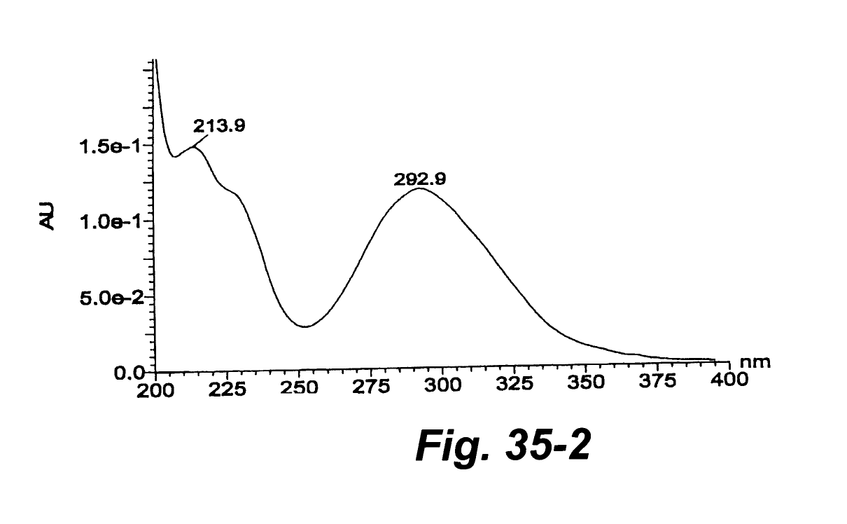

Kaempferol-7-neohesperidin (100 μg) and caffeic acid (100 μg) were dissolved in purified water (200 μL), and the mixture was warmed in an incubator at 50° C. for 1 minute. Laccase DAIWA Y120 (10 Amano Enzyme Inc.) prepared to 5 mg/mL was added and the mixture was stirred for 1 minute. Ethanol (200 μL) was further added to quench the enzyme reaction. This reaction mixture (1 μL) was analyzed by LC/MS, and two components having a molecular weight of 772, as well as m/z 465, which is a fragment peak of aglycone without sugar, were confirmed in the fractions with retention time 30.7 minutes and 32.1 minutes. UV spectrum was simultaneously measured by a photodiode array detector (see FIGS. 33 to 35). As a result of the UV spectrum analysis of each component, the absorption around 370 nm derived from ring C of kaempferol-7-neohesperidin disappeared, and only the absorption around 280 nm derived from ring A and ring B was strongly detected, which confirms that the structure of (compound 31) or (compound 32) was produced. The apparatus and measurement conditions of LC/MS and photodiode array detector are shown below.

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid and 10% acetonitrile in water, SOLUTION B solution of 0.05% trifluoroacetic acid and 80% acetonitrile in water

### Synthesis Example 13

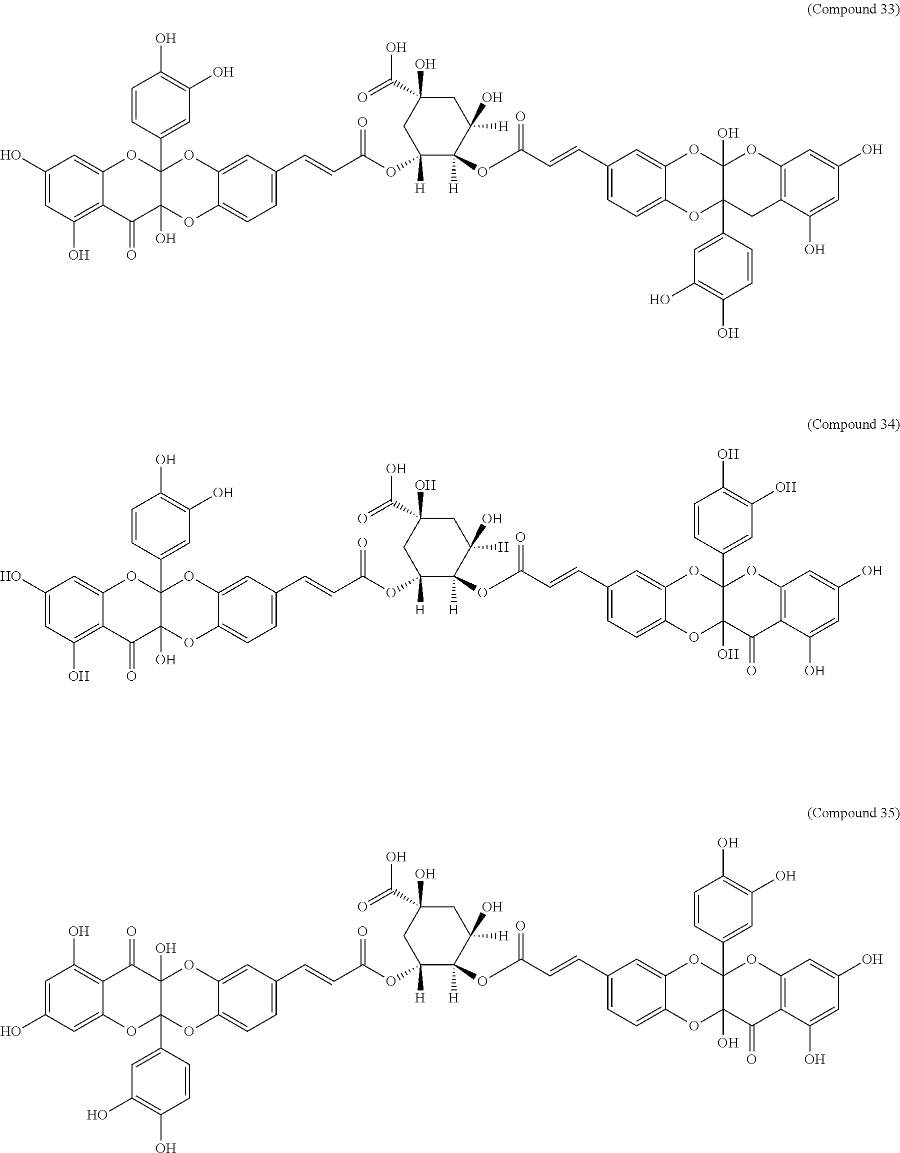

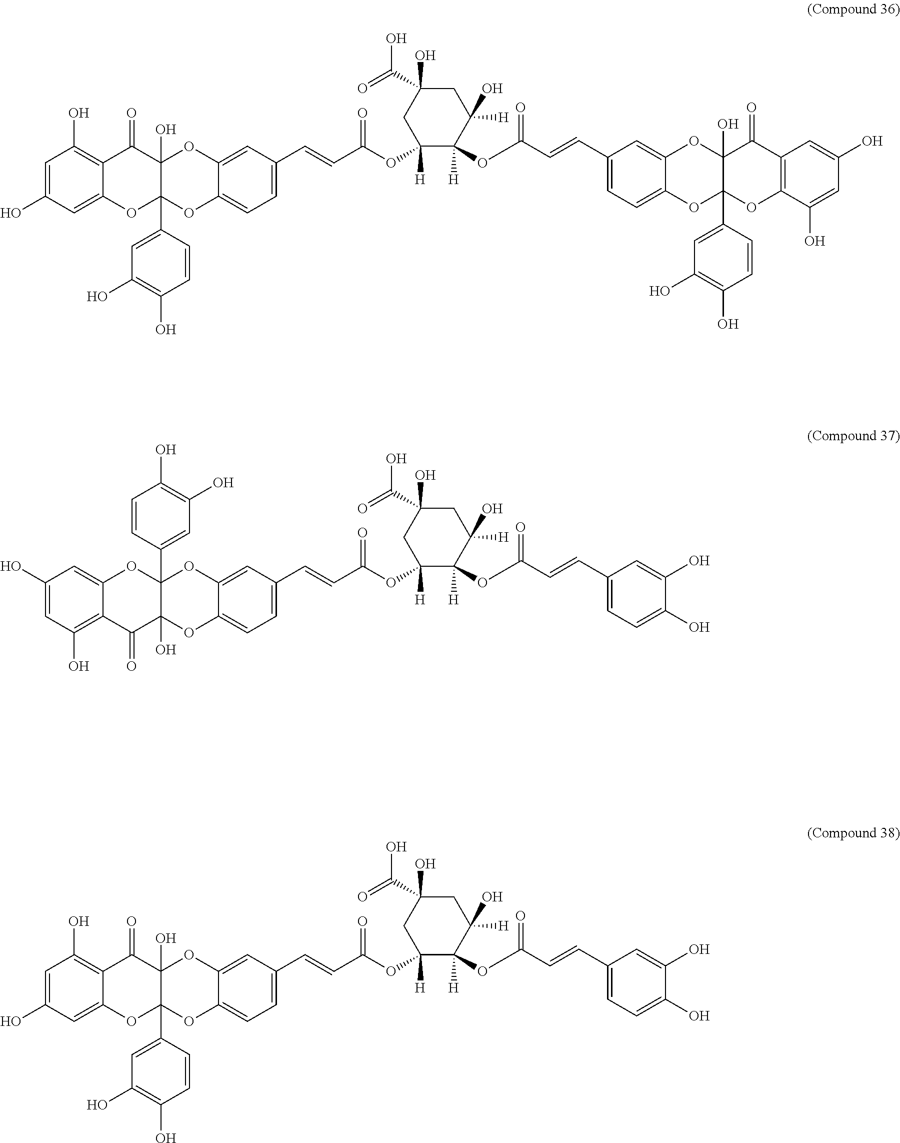

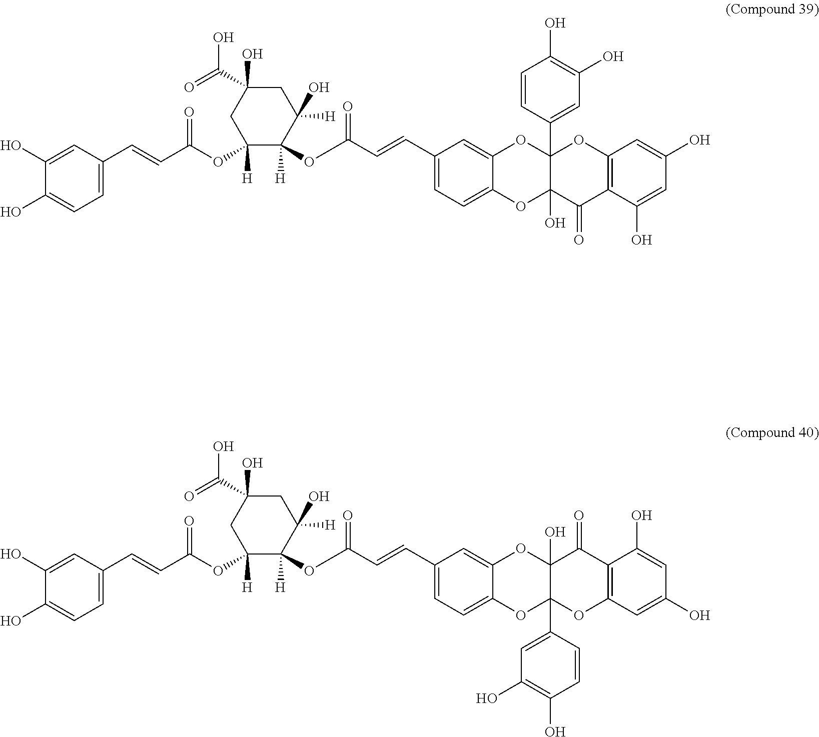

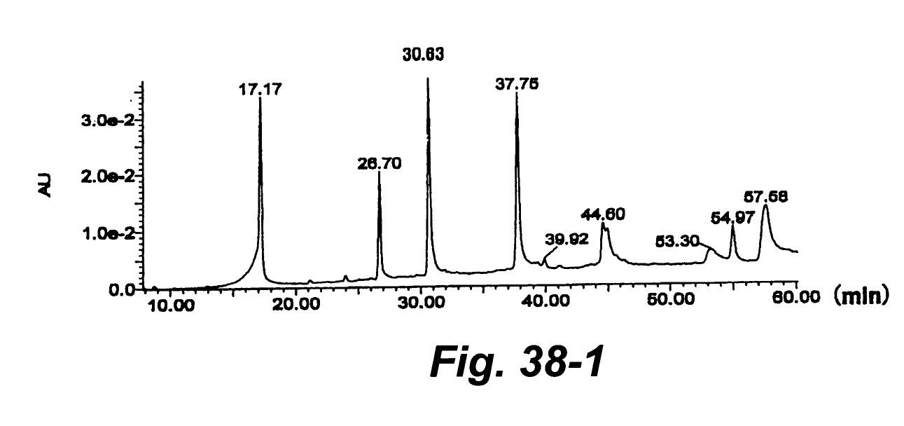

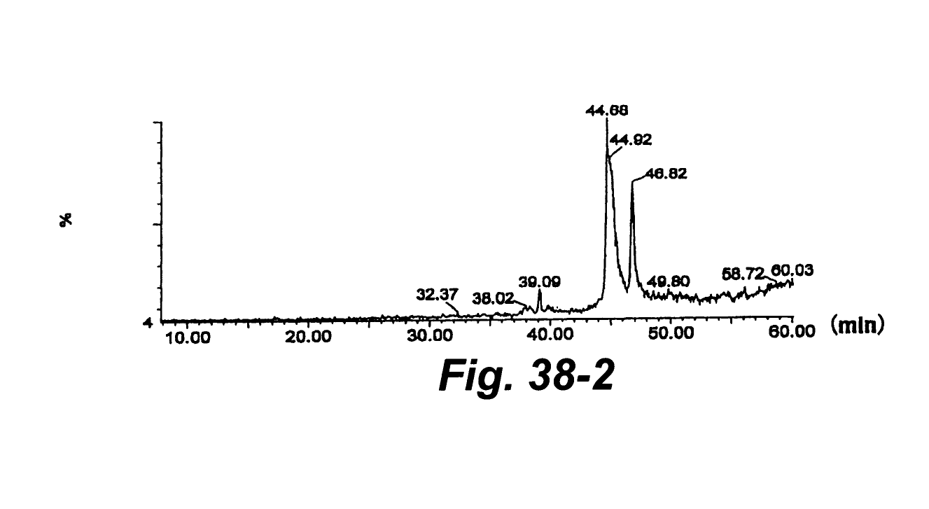

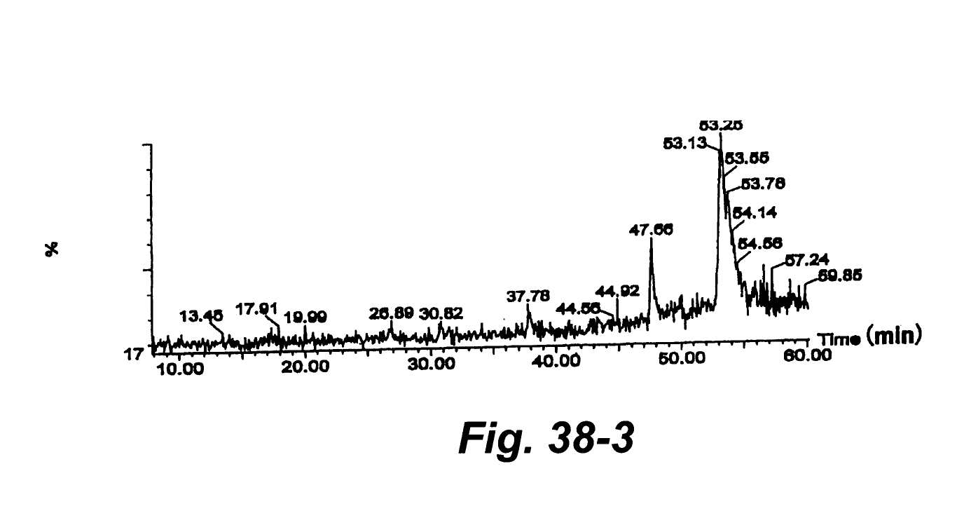

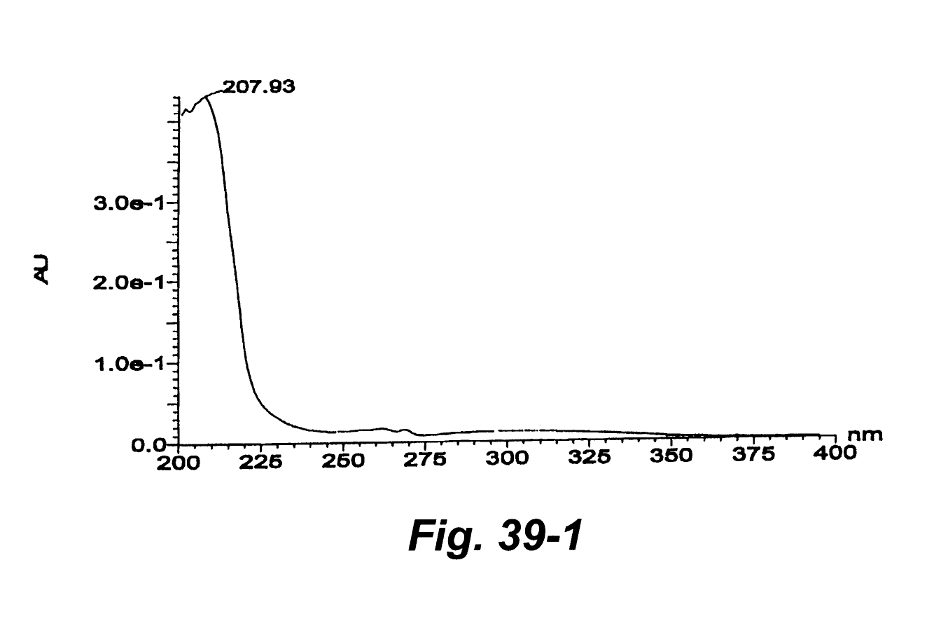

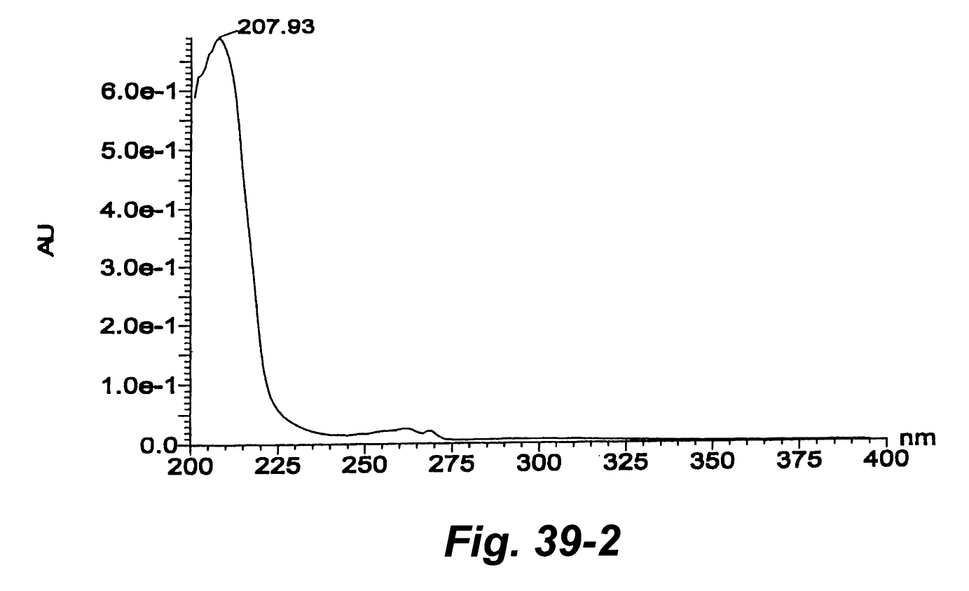

Quercetin 2 hydrate (100 μg) and 3,4-dicaffeoylquinic acid (100 μg) were dissolved in purified water (200 μL), and the mixture was warmed in an incubator at 50° C. for 1 minute. Laccase DAIWA Y120 (10 μL, Amano Enzyme Inc.) prepared to 5 mg/mL was added and the mixture was stirred for 1 minute. Ethanol (200 μL) was further added to quench the enzyme reaction. This reaction mixture (1 μL) was analyzed by LC/MS in selective ion analysis mode, and 4 components having a molecular weight of 816 could be confirmed in the fractions with retention time 44.6 minutes and 53.3 minutes. In addition, as a result of the UV spectrum analysis of each component simultaneously measured by a photodiode array detector (detection wavelength: 300 nm), the absorption around 370 nm derived from ring C of quercetin disappeared, and only the absorption around 280 nm derived from ring A and ring B was strongly detected, which confirms that the structures from (compound 37) to (compound 40) were produced (see FIG. 38 and FIG. 39).

Furthermore, as a result of the analysis by LC/MS in selective ion analysis mode, a component having a molecular weight of 1116 could be confirmed. Then, as a result of the UV spectrum analysis of each component, the absorption around 370 nm derived from ring C of quercetin disappeared, and only the absorption around 280 nm derived from ring A and ring B was strongly detected, which confirms that the structures from (compound 33) to (compound 36) were produced (see FIG. 38 and FIG. 39). The apparatus and measurement conditions of LC/MS and photodiode array detector are shown below.

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

m/z817, m/z1117 selective detection mode

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minutes, solvent: SOLUTION A solution of 0.05% trifluoroacetic acid and 10% acetonitrile in water, SOLUTION B solution of 0.05% trifluoroacetic acid and 80% acetonitrile in water

### Synthesis Reference Example 1

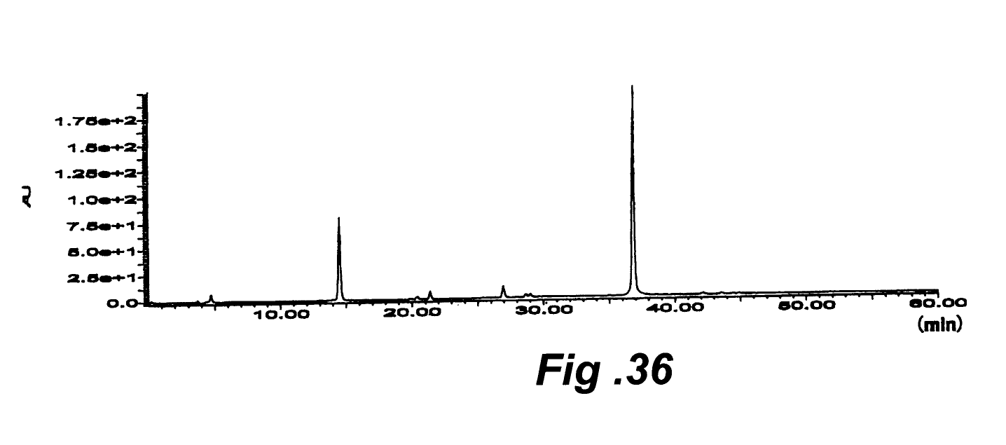

Luteolin (100 μg) and caffeic acid (100 μg) were dissolved in purified water (200 μL), and the mixture was warmed in an incubator at 50° C. for 1 minute. Laccase DAIWA Y120 (10 μL, Amano Enzyme Inc.) prepared to 5 mg/mL was added and the mixture was stirred for 1 minute. Ethanol (200 μL) was further added to quench the enzyme reaction. This reaction mixture (1 μL) was analyzed by LC/MS, and detection of a component having a molecular weight of 464, which was expected to be produced when luteolin and caffeic acid were bonded, was tried, but the component could not be confirmed. The peaks of luteolin (retention time 36.9 minutes, molecular weight 286) and caffeic acid (retention time 14.5 minutes, molecular weight 180) were merely detected (see FIG. 36). Thus, it has been confirmed that a reaction does not take place when luteolin is used as a substrate. The apparatus and measurement conditions of LC/MS and photodiode array detector are shown below.

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL, flow rate: 0.2 mL/minute, solvent: SOLUTION A solution of 0.05% trifluoroacetic acid and 10% acetonitrile in water, SOLUTION B solution of 0.05% trifluoroacetic acid and 80% acetonitrile in water

### Synthesis Reference Example 2

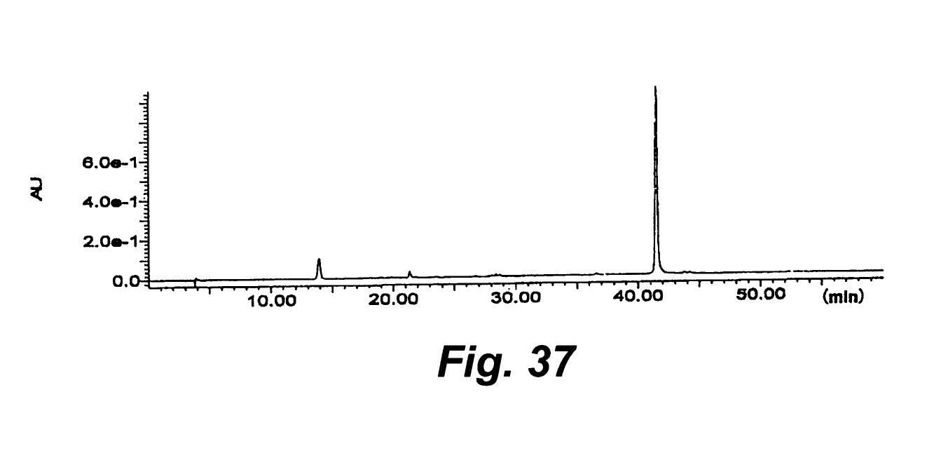

Apigenin (100 μg) and caffeic acid (100 μg) were dissolved in purified water (200 μL), and the mixture was warmed in an incubator at 50° C. for 1 minute. Laccase DAIWA Y120 (10 μL, Amano Enzyme Inc.) prepared to 5 mg/mL was added and the mixture was stirred for 1 minute. Ethanol (200 was further added to quench the enzyme reaction. This reaction mixture (1 μl) was analyzed by LC/MS, and detection of a component having a molecular weight of 448, which was expected to be produced when apigenin and caffeic acid were bonded, was tried, but the component could not be confirmed. The peaks of apigenin (retention time 41.7 minutes, molecular weight 270) and caffeic acid (retention time 13.9 minutes, molecular weight 180) were merely detected (see FIG. 37). Thus, it has been confirmed that a reaction does not take place when apigenin is used as a substrate. The apparatus and measurement conditions of LC/MS and photodiode array detector are shown below.

LC/MS analysis apparatus:

LC: Waters Alliance 2695

detector: Waters 2996 Photodiode array detector (manufactured by Waters)

detector: Waters Quattro micro API (manufactured by Waters) ionization method: ESI

LC/MS analysis conditions:

column: CAPCELL PAK AQ S-3 μm, 2×250 mm (manufactured by Shiseido Co., Ltd.)

gradient conditions: 0 minute (SOLUTION A/SOLUTION B=100:0), 100 minutes (SOLUTION A/SOLUTION B=0:100), injection volume: 1 μL flow rate: 0.2 mL/minute, solvent: SOLUTION A solution of 0.05% trifluoroacetic acid and 10% acetonitrile in water, SOLUTION B solution of 0.05% trifluoroacetic acid and 80% acetonitrile in water

### Physiological Activity Test Example.

The lipase inhibitory activity of the compounds obtained in the above-mentioned Synthesis Example 1, Synthesis Example 2, Synthesis Example 3, Synthesis Example 5, and Synthesis Example 13, and the enzyme-treated reaction crude products obtained by reacting each reaction substrate compound (0.3 mol) by the methods of Synthesis Example 1, Synthesis Example 2, and Synthesis Example 5 (i.e., unpurified resultant product) was measured. The lipase inhibitory activity was measured using pancreatic lipase derived from swine (manufactured by Sigma Ltd.) and 4-methylumbelliferyl oleate (4-MUO) (manufactured by Sigma Ltd.) as a substrate under the following conditions. A test sample of the compound of each Synthesis Example was dissolved in dimethyl sulfoxide (DMSO) to 20 mM, test samples of the mixture (before reaction) and the reaction crude product were dissolved in dimethyl sulfoxide (DMSO) in an amount of 1.68 mg/mL (Synthesis Examples 1 and 2) and 2.43 mg/mL (Synthesis Example 5), respectively, as calculated based on the total weight of each reaction substrate compound charged. The solution (5 μL) and an enzyme solution (a 0.25 mg/mL solution of pancreatic lipase derived from swine in phosphoric acid-buffered saline (PBS), 45 μL) were mixed for 5 minutes, a substrate solution (100 mM 4-MUO PBS solution, 50 μL) was added, and the mixture was reacted at 37° C. for 30 minutes. Immediately thereafter, the fluorescence intensity due to the decomposition of 4-MUO was measured at excitation wavelength 355 nm, and fluorescence wavelength 460 nm. Using DMSO (5 μL) as a control, the fluorescence intensity of Synthesis Example compounds and control compounds each without addition of enzyme but with addition of PBS alone was measured and used as a blank. The lipase inhibitory activity of each sample is shown in the following Table 3 to Table 5 as lipase inhibitory activity (%) at 250 μm or 84 μg/mL, or IC₅₀ (μM) or IC₅₀ (μg/mL). The lipase inhibitory activity (%) was calculated by the following formula. The fluorescence detector used was SPECTRA MAX GEMINI EM (Molecular Devices).

lipase inhibitory activity(%)={1−(EMₛ−EM_{sb})/(EM_{c}−EM_{cb})}×100

EMₛ: fluorescence intensity of 4-MUO decomposed product with addition of Synthesis Example compound

EM_{sb}: fluorescence intensity of blank with addition of Synthesis Example compound

EM_{c}: fluorescence intensity of 4-MUO decomposed product with addition of control compound

EM_{cb}: fluorescence intensity of blank with addition of control compound

TABLE 1
¹H-NMR analysis results (δ).

| | compound 1 | compound 1 | compound 3 | compound 5 | compound 5 |
|---|---|---|---|---|---|
| posi- | or | or | or | or | or |
| tion | compound 2 | compound 2 | compound 4 | compound 6 | compound 6 |
| 6 | 5.95 (1 H, d, | 5.92 (1 H, d, | 5.94 (1 H, d, | 5.92 (1 H, d, | 5.92 (1 H, d, |
| | J = 2.1 Hz) | J = 2.1 Hz) | J = 2.1 Hz) | J = 2.1 Hz) | J = 2.0 Hz) |
| 8 | 5.96 (1 H, d, | 5.93 (1 H, d, | 5.96 (1 H, d, | 5.93 (1 H, d, | 5.93 (1 H, d, |
| | J = 2.1 Hz) | J = 2.1 Hz) | J = 2.1 Hz) | J = 2.1 Hz) | J = 2.0 Hz) |
| 2′ | 7.23 (1 H, d, | 7.22 (1 H, d, | 7.27 (1 H, d, | 7.21 (1 H, d, | 7.22 (1 H, d, |
| | J = 2.3 Hz) | J = 2.3 Hz) | J = 2.3 Hz) | J = 2.3 Hz) | J = 2.2 Hz) |
| 5′ | 6.71 (1 H, d, | 6.68 (1 H, d, | 6.69 (1 H, d, | 6.68 (1 H, d, | 6.68 (1 H, d, |
| | J = 8.5 Hz) | J = 8.5 Hz) | J = 8.5 Hz) | J = 8.84 Hz) | J = 8.5 Hz) |
| 6′ | 7.06 (1 H, d, | 7.04 (1 H, d, | 7.09 (1 H, d, | 7.03 (1 H, d, | 7.04 (1 H, d, |
| | J = 8.5, 2.3 Hz) | J = 8.5, 2.3 Hz) | J = 8.5, 2.3 Hz) | J = 8.4, 2.3 Hz) | J = 8.5, 2.2 Hz) |
| 2″ | 6.39 (1 H, d, | 6.45 (1 H, d, | — | 6.41 (1 H, d, | 6.43 (1 H, d, |
| | J = 15.9 Hz) | J = 15.9 Hz) | | J = 15.9 Hz) | J = 15.2 Hz) |
| 3″ | 7.61 (1 H, d, | 7.62 (1 H, d, | 7.14 (1 H, d, | 7.63 (1 H, d, | 7.65 (1 H, d, |
| | J = 15.9 Hz) | J = 15.9 Hz) | J = 1.6 Hz) | J = 15.9 Hz) | J = 15.2 Hz) |
| 4″ | — | — | — | — | — |
| 5″ | 7.25(1 H, d, | 7.32 (1 H, d, | — | 7.24 (1 H, d, | 7.35 (1 H, m) |
| | J = 2.0 Hz) | J = 1.9 Hz) | | J = 2.0 Hz) | |
| 6″ | — | — | — | — | — |
| 7″ | — | — | 7.24 (1 H, d, | — | — |
| | | | J = 1.6 Hz) | | |
| 8″ | 7.09 (1 H, d, | 6.98 (1 H, d, | — | 7.07 (1 H, d, | 7.00 (1 H, d, |
| | J = 8.3 Hz) | J = 8.4 Hz) | | J = 8.4 Hz) | J = 8.3 Hz) |
| 9″ | 7.28 (1 H, d, | 7.24 (1 H, d, | — | 7.27 (1 H, d, | 7.26 (1 H, m) |
| | J = 8.3, 2.0 Hz) | J = 8.4, 1.9 Hz) | | J = 8.4, 2.0 Hz) | |
| 2′′′ | — | — | — | 2.20 (2 H, m) | 2.20 (2 H, m) |
| 3′′′ | — | — | — | 5.33 (1 H, m) | 5.33 (1 H, m) |
| 4′′′ | | | | 3.72 (1 H, dd, | 3.73 (1 H, m) |
| | — | — | — | J = 3.1, 8.4 Hz) | |
| 5′′′ | — | — | — | 4.16 (1 H, d, | 4.17 (1 H, m) |
| | | | | J = 5.1 Hz) | |
| 6′′′ | — | — | — | 2.04 (2 H, m) | 2.04 (2 H, m) |

| posi- | compound 15 | compound 15 | compound 15 | compound 15 |
|---|---|---|---|---|
| tion | or 16 | or 16 | or 16 | or 16 |
| 6 | 6.00 (1 H, s) | 5.96 (1 H, s) | 5.98 (1 H, s) | 5.98 (1 H, s) |
| 8 | 6.00 (1 H, s) | 5.96 (1 H, s) | 5.97 (1 H, s) | 5.97 (1 H, s) |
| 2′ | 7.19 (1 H, d, | 7.13 (1 H, d, | 7.14 (1 H, d, | 7.13 (1 H, d, |
| | J = 2.2 Hz) | J = 2.2 Hz) | J = 2.3 Hz) | J = 2.0 Hz) |
| 5′ | 6.73 (1 H, d, | 6.68 (1 H, d, | 6.68 (1 H, d, | 6.68 (1 H, d, |
| | J = 8.4 Hz) | J = 8.4 Hz) | J = 8.0 Hz) | J = 8.4 Hz) |
| 6′ | 6.97 (1 H, dd, | 6.91 (1 H, dd, | 6.92 (1 H, dd, | 6.92 (1 H, dd, |
| | J = 2.2, 8.4 Hz) | J = 2.2, 8.4 Hz) | J = 2.3, 8.0 Hz) | J = 2.0, 8.4 Hz) |
| 2″ | 4.61 (1 H, d, | 4.62 (1 H, d, | 4.60 (1 H, d, | 4.59 (1 H, d, |
| | J = 8.0 Hz) | J = 7.5 Hz) | J = 7.9 Hz) | J = 7.9 Hz) |
| 3″ | 3.92 (1 H, m) | 3.88 (1 H, m) | 3.91 (1 H, m) | 3.89 (1 H, m) |
| 4″ | 2.79 (1 H, dd, | 2.68 (1 H, dd, | 2.74 (1 H, dd, | 2.75 (1 H, dd, |
| | J = 5.4, 16.0 Hz) | J = 5.0, 15.9 Hz) | J = 5.3, 15.9 Hz) | J = 5.3, 16.0 Hz) |
| 5″ | 2.42 (1H, dd, | 2.38 (1H, dd, | 2.39 (1H, dd, | 2.39 (1 H, dd, |
| | J = 8.6, 16.0 Hz) | J = 8.0, 15.9 Hz) | J = 8.5, 15.9 Hz) | J = 8.4, 16.0 Hz) |
| 6″ | 5.75 (1 H, d, | 5.72 (1 H, d, | 5.72 (1 H, d, | 5.73 (1 H, d, |
| | J = 1.6 Hz) | J = 2.0 Hz) | J = 2.2 Hz) | J = 1.6 Hz) |
| 7″ | — | — | — | — |
| 8″ | 5.96 (1 H, d, | 5.91(1 H, d, | 5.92 (1 H, d, | 5.92 (1 H, d, |
| | J = 1.7 Hz) | J = 2.0 Hz) | J = 2.2 Hz) | J = 1.7 Hz) |
| 9″ | — | — | — | — |
| 2′′′ | 7.03 (1 H, m) | 6.98 (1 H, d, | 7.04 (1 H, d, | 7.05 (1H, m) |
| | | J = 1.5 Hz) | J = 1.5 Hz) | |
| 3′′′ | — | — | — | — |
| 4′′′ | — | — | — | — |
| 5′′′ | 7.11 (1 H,d, | 7.06 (1 H, d, | 6.97 (1 H, d, | 6.97 (1 H, d, |
| | J = 8.2 Hz) | J = 8.4 Hz) | J = 8.0 Hz) | J = 8.3 Hz) |
| 6′′′ | 7.08 (1 H, dd, | 7.00 (1 H, m) | 7.00 (1 H, dd, | 7.00 (1 H, m) |
| | J = 1.5, 8.5 Hz) | | J = 1.5 ,8.0 Hz) | |

TABLE 2
¹³C-NMR analysis results (δ).

| | compound 1 | compound 1 | compound 3 | compound 5 | compound 5 |
|---|---|---|---|---|---|
| posi- | or | or | or | or | or |
| tion | compound 2 | compound 2 | compound 4 | compound 6 | compound 6 |
| 2 | 102.4 | 101.8 | 102.2 | 102.4 | 102.2 |
| 3 | 92.3 | 92.1 | 92.2 | 92.3 | 92.5 |
| 4 | 190.3 | 189.8 | 190.4 | 190.3 | 190.2 |
| 5 | 161.6 | 161.2 | 161.5 | 161.6 | 161.6 |
| 6 | 98.3 | 97.9 | 98.4 | 98.4 | 98.4 |
| 7 | 170.1 | 169.7 | 169.8 | 170.1 | 170.1 |
| 8 | 97.7 | 97.3 | 97.8 | 97.7 | 97.7 |
| 9 | 165.8 | 165.4 | 165.8 | 165.8 | 165.8 |
| 10 | 101.7 | 101.3 | 101.7 | 101.7 | 101.7 |
| 1′ | 127.2 | 126.9 | 127.1 | 127.2 | 127.2 |
| 2′ | 117.2 | 116.8 | 117.5 | 117.2 | 117.2 |
| 3′ | 146.2 | 145.8 | 146.2 | 146.2 | 146.2 |
| 4′ | 148.4 | 148.0 | 148.5 | 148.4 | 148.4 |
| 5′ | 115.8 | 115.5 | 115.9 | 115.9 | 115.9 |
| 6′ | 121.7 | 121.3 | 121.9 | 121.7 | 121.7 |
| 1″ | 170.8 | 170.5 | 170.1 | 168.5 | 168.6 |
| 2″ | 118.8 | 118.3 | 125.9 | 118.5 | 118.3 |
| 3″ | 146.0 | 145.7 | 111.8 | 146.1 | 146.1 |
| 4″ | 131.3 | 130.7 | 147.6 | 131.3 | 131.1 |
| 5″ | 118.3 | 117.7 | 135.5 | 118.4 | 118.1 |
| 6″ | 143.1 | 142.4 | 143.3 | 143.1 | 142.7 |
| 7″ | 144.4 | 144.8 | 112.8 | 144.5 | 145.0 |
| 8″ | 119.1 | 119.0 | — | 119.1 | 119.4 |
| 9″ | 124.5 | 124.4 | — | 124.7 | 124.9 |
| 1″ | — | — | — | 73.8 | 76.6 |
| 2′′′ | — | — | — | 39.2 | 39.2 |
| 3′′′ | — | — | — | 71.7 | 71.7 |
| 4′′′ | — | — | — | 72.6 | 72.6 |
| 5′′′ | — | — | — | 73.8 | 73.8 |
| 6′′′ | — | — | — | 38.6 | 38.6 |
| 7′′′ | — | — | — | 177.4 | 170.1 |

| posi- | compound 15 | compound 15 | compound 15 | compound 15 |
|---|---|---|---|---|
| tion | or 16 | or 16 | or 16 | or 16 |
| 2 | 100.5 | 100.5 | 99.0 | 100.5 |
| 3 | 90.6 | 90.7 | 89.2 | 90.7 |
| 4 | 188.1 | 188.1 | 186.6 | 188.1 |
| 5 | 100.2 | 100.2 | 98.7 | 100.2 |
| 6 | 159.7 | 159.7 | 158.2 | 159.7 |
| 7 | 96.5 | 98.5 | 95.0 | 96.5 |
| 8 | 168.1 | 168.1 | 166.6 | 168.1 |
| 9 | 97.4 | 97.4 | 95.8 | 97.4 |
| 10 | 163.4 | 163.4 | 161.9 | 163.4 |
| 1′ | 125.1 | 125.1 | 123.6 | 125.1 |
| 2′ | 116.1 | 116.1 | 114.6 | 116.1 |
| 3′ | 147.2 | 147.2 | 145.7 | 147.2 |
| 4′ | 145.0 | 145.0 | 143.5 | 145.0 |
| 5′ | 115.1 | 115.1 | 113.6 | 115.1 |
| 6′ | 119.6 | 119.6 | 118.1 | 120.0 |
| 1″ | 81.0 | 80.6 | 79.3 | 81.0 |
| 2″ | 66.6 | 66.5 | 65.1 | 66.6 |
| 3″ | 28.8 | 28.4 | 27.2 | 28.8 |
| 4″ | 156.9 | 156.9 | 155.4 | 156.9 |
| 5″ | 94.2 | 94.2 | 92.7 | 94.2 |
| 6″ | 156.5 | 156.5 | 155.0 | 156.6 |
| 7″ | 95.7 | 95.7 | 94.2 | 95.7 |
| 8″ | 155.6 | 155.5 | 154.0 | 155.6 |
| 9″ | 99.5 | 99.3 | 97.9 | 99.5 |
| 1′′′ | 134.9 | 134.9 | 133.1 | 134.6 |
| 2′′′ | 116.6 | 116.5 | 114.8 | 116.3 |
| 3′′′ | 140.6 | 140.6 | 139.0 | 140.5 |
| 4′′′ | 140.1 | 140.0 | 138.7 | 140.2 |
| 5′′′ | 116.8 | 116.9 | 115.8 | 117.2 |
| 6′′′ | 122.3 | 121.8 | 120.7 | 122.4 |
| 7′′′ | — | — | — | — |

TABLE 3
lipase inhibitory test.

| | | | lipase |
|---|---|---|---|
| | concen- | | inhibitory |
| sample | tration | | activity (%) |
| quercetin | 250 | μM | 64.3 |
| caffeic acid | 250 | μM | 14.0 |
| gallic acid | 250 | μM | 50.0 |
| (compound 1) or (compound 2) | 250 | μM | 95.5 |
| (compound 3) or (compound 4) | 250 | μM | 87.2 |
| mixture of quercetin and | 84 | μg/mL | 44.9 |
| caffeic acid (before reaction) | | | |
| mixture of quercetin and | 85 | μg/mL | 79.0 |
| gallic acid (before reaction) | | | |
| reaction crude product of | 84 | μg/mL | 93.2 |
| quercetin and caffeic acid | | | |
| reaction crude product of | 85 | μg/mL | 93.1 |
| quercetin and gallic acid | | | |

TABLE 4
lipase inhibitory test.

| sample | IC₅₀ (μM) |
|---|---|
| mixture of (compound 1) and (compound 2) | 11 |
| mixture of (compound 3) and (compound 4) | 23 |
| mixture of (compound 5) and (compound 6) | 11 |
| mixture of (compound 15) and (compound 16) | 17 |
| mixture of (compound 33) to (compound 36) | 12 |
| mixture of (compound 37) to (compound 40) | 25 |

TABLE 5
lipase inhibitory test.

| | sample | IC₅₀ (μg/mL) |
|---|---|---|
| Synthesis | mixture of quercetin and caffeic | 46 |
| Experimental | acid (before reaction) | |
| Example 1 | reaction crude product of | 36 |
| | quercetin and caffeic acid | |
| Synthesis | mixture of quercetin and gallic | 22 |
| Experimental | acid (before reaction) | |
| Example 2 | reaction crude product of | 16 |
| | quercetin and gallic acid | |
| Synthesis | mixture of quercetin and catechin | 88 |
| Experimental | (before reaction) | |
| Example 5 | reaction crude product of | 17 |
| | quercetin and catechin | |

From the above-mentioned results, it has been shown that the compound or resultant product of the present invention has a superior lipase inhibitory activity.

### INDUSTRIAL APPLICABILITY

The present invention can provide a novel compound superior in lipase inhibitory action. The compound is highly safe and can be ingested orally. Particularly, the compound prevents or improves postprandial hyperlipemia and lipid dysbolism caused by ingestion of high-fat diet, is further effective for the prophylaxis or improvement of obesity, and is expected to provide a prophylactic or improving effect on glucose metabolism disorder such as insulin resistance and increased blood insulin concentration. In addition, using the compound, moreover, dermatic diseases caused by bacterial lipase such as acne and the like and body odor can be prevented or improved by inhibiting lipase produced by skin indigenous bacteria in human such as Propionibacterium acnes and the like.

Although the present invention have been presented or described by referring to preferred embodiments of this invention, it will, however, be understood by those of ordinary skill in the art that various modifications may be made to the forms and details without departing from the scope of the invention as set forth in the appended claims.

All patents, patent publications and other publications indicated or cited in the Specification are hereby incorporated in their entireties by reference.

Where a numerical limit or range is stated herein, the endpoints are included. Also, all values and subranges within a numerical limit or range are specifically included as if explicitly written out.

1. A compound represented by formula (I) 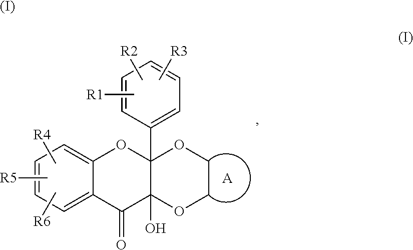
wherein:
R1, R2, R3, R4, R5, and R6 may be the same or different and each is
a hydrogen atom,
a hydroxyl group,
alkoxy group, or
a sugar residue consisting of 1 or 2 sugars selected from the group consisting of glucose, rhamnose, fructose and galactose;
ring A is a ring selected from the group consisting of the following formulas (II-a)-(II-i) 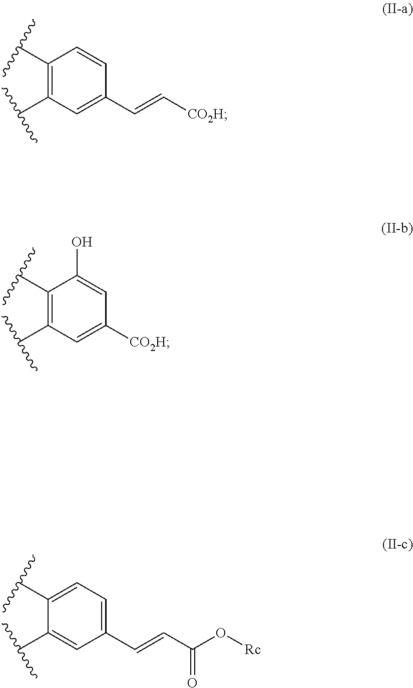
wherein in formula (II-c),
Rc is a group represented by the following formula 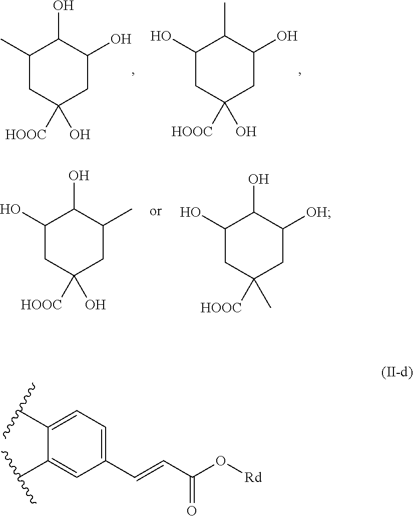
wherein in formula (II-d),
Rd is a group represented by the following formula 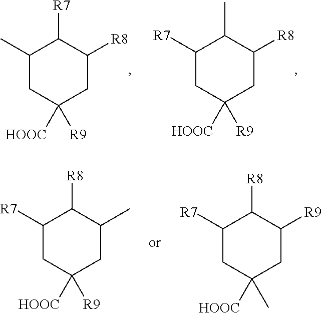
wherein
any one group of R7, R8 and R9 is a group represented by the formula: 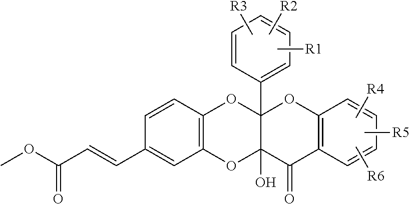
wherein R1, R2, R3, R4, R5 and R6 are as defined above, a group represented by the formula: 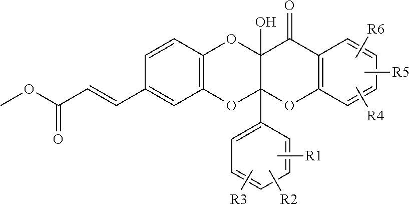
wherein R1, R2, R3, R4, R5 and R6 are as defined above, or a group represented by the formula: 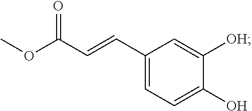
and
two groups of the remaining R7, R8 and R9 are hydroxyl groups; 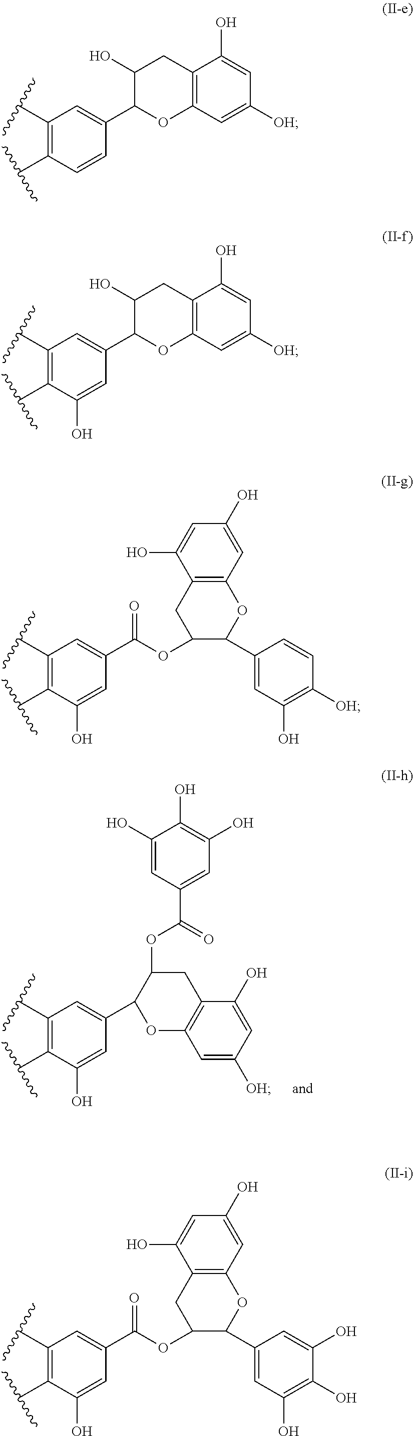
wherein the above-mentioned rings are each optionally condensed with the adjacent dioxane ring in any orientation.

2. A compound according to claim 1, wherein R1, R2, R3, R4, R5 and R6 may be the same or different and each is
a hydrogen atom,
a hydroxyl group,
a methoxy group, or
a sugar residue consisting of 1 or 2 sugars selected from the group consisting of glucose, rhamnose and galactose.

3. A compound according to claim 1, which is represented by formula (I′) 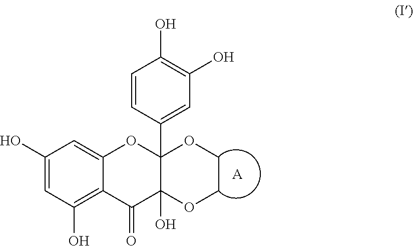
wherein ring A is a ring selected from the group consisting of the following formulas (II-a′)-(II-g′) 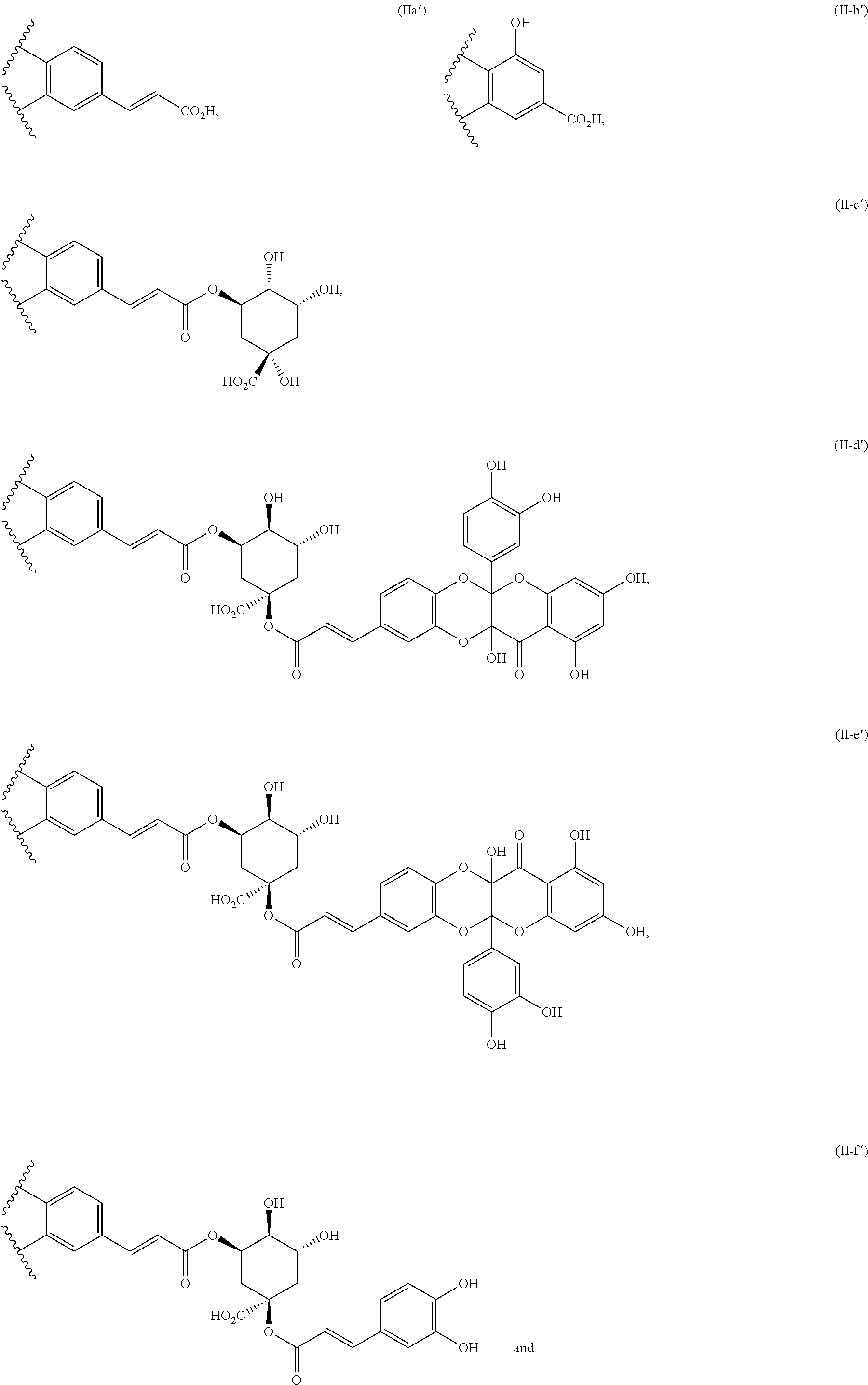 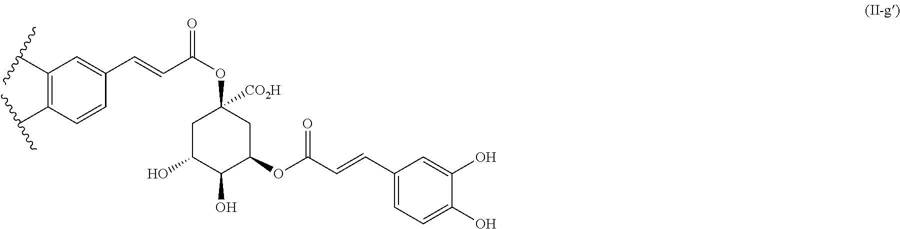
wherein the above-mentioned rings are each optionally condensed with the adjacent dioxane ring in any orientation.

4. A product, which is obtained by reacting one or more kinds of a flavonol compound with one or more kinds of a component selected from the group consisting of caffeic acid, gallic acid, chlorogenic acid, dicaffeoylquinic acid, catechin, gallocatechin, catechin gallate, and gallocatechin gallate in the presence of polyphenoloxydase.

5. A product according to claim 4, wherein said one or more kinds of flavonol compound and said one or more kinds of component selected from the group consisting of caffeic acid, gallic acid, chlorogenic acid, dicaffeoylquinic acid, catechin, gallocatechin, catechin gallate, and gallocatechin gallate are extracted from natural materials.

6. A product, which is obtained by reacting one or more kinds of a flavonol compound represented by the formula (III) 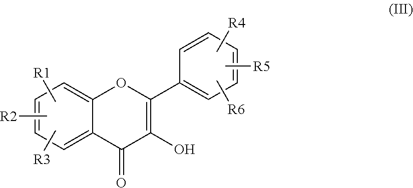
wherein R1, R2, R3, R4, R5, and R6 may be the same or different and each is
a hydrogen atom,
a hydroxyl group,
alkoxy group, or
a sugar residue consisting of 1 or 2 sugars selected from the is group consisting of glucose, rhamnose, fructose and galactose; with
one or more kinds of a components selected from the group consisting of a compound represented by the following structural formula 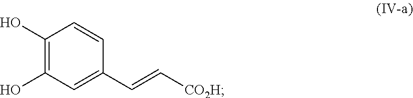
a compound represented by 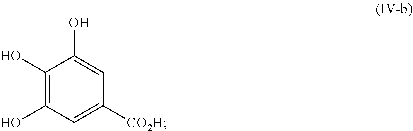
a compound represented by 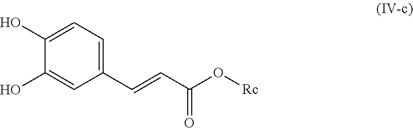
in the formula (IV-c),
Rc is a group represented by the following formula 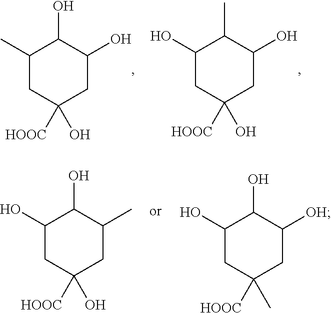
a compound represented by 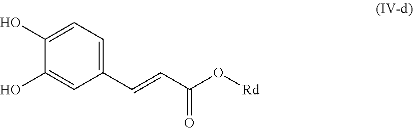
in the formula (IV-d),
Rd is a group represented by the following formula 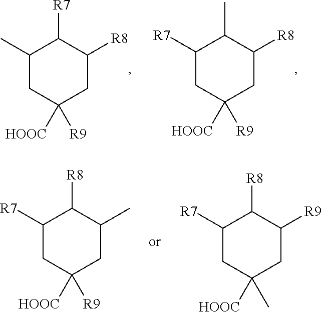
wherein,
any one group of R7, R8 and R9 is a group represented by the formula: 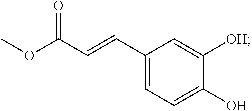
and
two groups of the remaining R7, R8 and R9 are hydroxyl groups;
a compound represented by 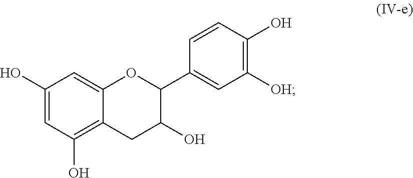
a compound represented by 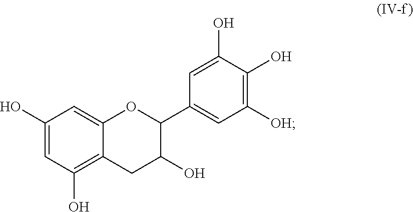
a compound represented by 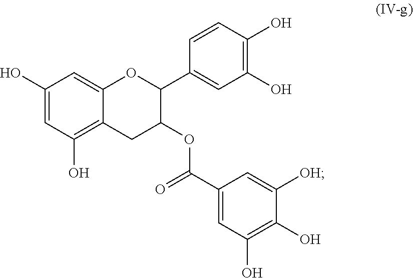
and
a compound represented by 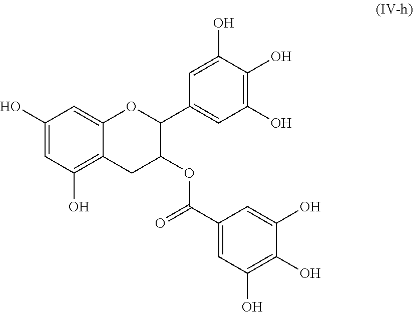
in the presence of polyphenoloxydase.

7. A product according to claim 4, which is obtained by reacting quercetin with one or more kinds of a component selected from the group consisting of caffeic acid, gallic acid, chlorogenic acid, and dicaffeoylquinic acid in the presence of polyphenoloxydase.

8. A composition for food or drink, comprising a compound according to claim 1.

9. A composition for food or drink according to claim 8, which is a tea drink, beverage, or health food.

10. A composition for food or drink, comprising a product according to claim 4.

11. A composition for food or drink according to claim 10, which is a tea drink, beverage, or health food.

12. A composition for food or drink, comprising a product according to claim 6.

13. A composition for food or drink according to claim 12, which is a tea drink, beverage or health food.

14. A method of inhibiting lipase, comprising giving an effective amount of a compound according to claim 1 to a subject in need thereof.

15. A method of inhibiting lipase, comprising giving an effective amount of a product according to claim 4 to a subject in need thereof.

16. A method of inhibiting lipase, comprising giving an effective amount of a product according to claim 6 to a subject in need thereof.

17. A method of suppressing fat absorption, comprising giving an effective amount of a compound according to claim 1 to a subject in need thereof.

18. A method of suppressing fat absorption, comprising giving an effective amount of a product according to claim 4 to a subject in need thereof.

19. A method of suppressing fat absorption, comprising giving an effective amount of a product according to claim 6 to a subject in need thereof.

20. A method of producing a compound according to claim 1, comprising subjecting one or more kinds of a flavonol compound represented by formula (III) 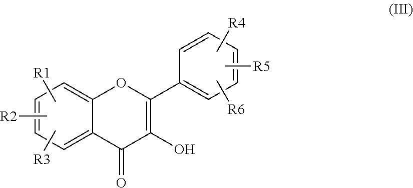
wherein R1, R2, R3, R4, R5, and R6 may be the same or different and each is
a hydrogen atom,
a hydroxyl group,
alkoxy group, or
a sugar residue consisting of 1 or 2 sugars selected from the group consisting of glucose, rhamnose, fructose and galactose;
and one or more kinds of a compound selected from the group consisting of
a compound represented by the following structural formula 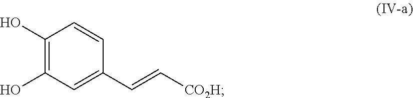
a compound represented by 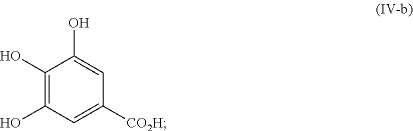
a compound represented by 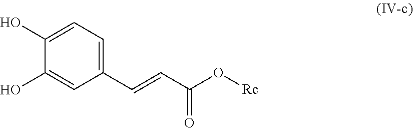
in the formula (IV-c),
Rc is a group represented by the following formula 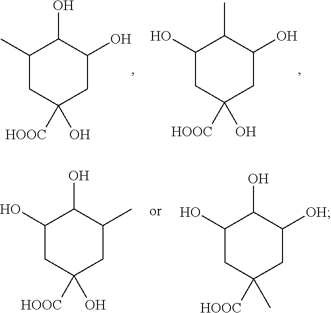
a compound represented by 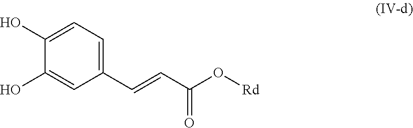
in the formula (IV-d),
Rd is a group represented by the following formula 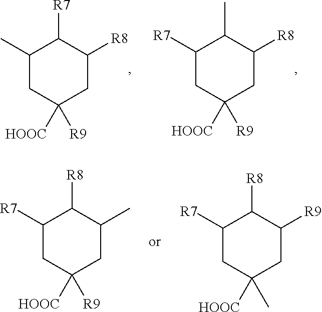
wherein
any one group of R7, R8 and R9 is a group represented by the formula: 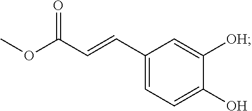
and
two groups of the remaining R7, R8 and R9 are hydroxyl groups;
a compound represented by 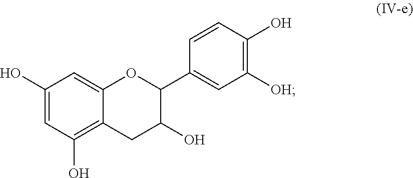
a compound represented by 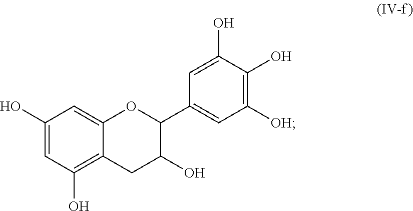
a compound represented by 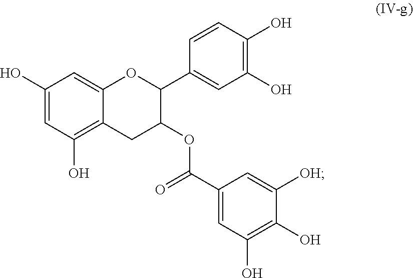
and
a compound represented by 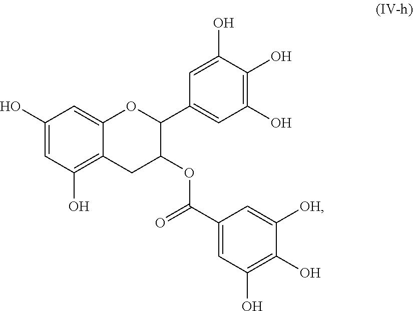
to an oxidation polymerization reaction in the presence of polyphenoloxydase.

21. A method of producing a compound according to claim 3, comprising subjecting one or more kinds of a flavonol compound represented by the formula (III′) 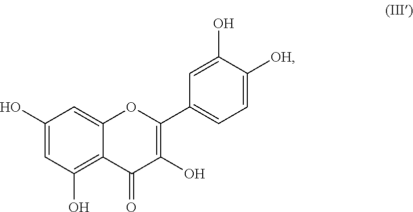
and one or more kinds of a compound selected from the group consisting of a compound represented by the following structural formula 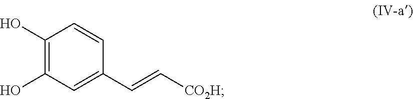
a compound represented by 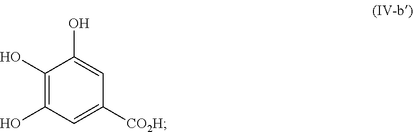
a compound represented by 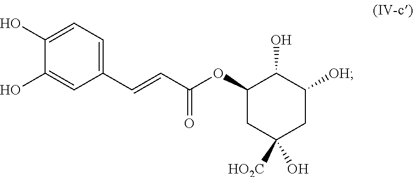
and
a compound represented by 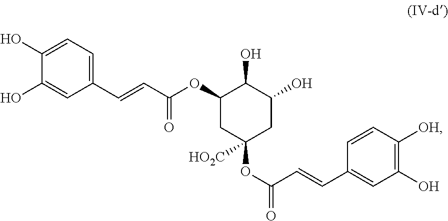
to an oxidation polymerization reaction in the presence of polyphenoloxydase.
